(19) United States
(12) Patent Application Publication
(10) Pub. No.: US 2026/0256746 A1
(43) Pub. Date: Sep. 3, 2026

(54) TRIAZOLES FOR USE IN THE TREATMENT OF OCULAR DISEASES

(71) Applicants: MIRAMOON PHARMA, S.L., Donostia-San Sebastián, Gipuzkoa (ES); ADMINISTRACIÓN GENERAL DE LA COMUNIDAD AUTÓNOMA DE EUSKADI, Vitoria-Gasteiz, Álava (ES); UNIVERSIDAD DEL PAÍS VASCO/EUSKAL HERRIKO UNIBERTSITATEA, Leioa, Vizcaya (ES)

(72) Inventors: Jesús María AIZPURUA IPARRAGUIRRE, Leioa, Biskaia (ES); Ainara VALLEJO ILLARRAMENDI, Leioa, Bizkaia (ES); Maialen SAGARTZAZU AIZPURUA, Leioa, Bizkaia (ES); José Ignacio MIRANDA MURUA, Leioa, Biskaia (ES); Adolfo José LÓPEZ DE MUNAIN ARREGI, Donostia-San Sebastián, Gipuzkoa (ES); Javier RUIZ EDERRA, Donostia-San Sebastián, Gipuzkoa (ES); Pablo FERRÓN CELMA, Donostia-San Sebastián, Gipuzkoa (ES)

(21) Appl. No.: 18/877,594

(22) PCT Filed: Jun. 22, 2023
(86) PCT No.: PCT/EP2023/067005

§ 371 Date: Dec. 20, 2024

(30) Foreign Application Priority Data
Jun. 23, 2022 (EP) 22382594.4

(51) Int. Cl. A61K 31/4192; A61K 9/00; A61K 31/454; A61K 31/5377; A61K 47/36; A61P 27/02; C07D 249/04; C07D 405/12

(52) U.S. Cl. CPC A61K 31/4192; A61K 9/0048; A61K 31/454; A61K 31/5377; A61K 47/36; A61P 27/02; C07D 249/04; C07D 405/12

(57) ABSTRACT

The present invention relates to substituted 1,2,3-triazoles of formula (I) useful for the treatment of ocular diseases, in particular those related to retinal degeneration. The invention also relates to pharmaceutical compositions to treat retinal diseases via topic administration.

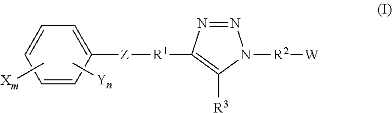

### FIELD OF THE INVENTION

The present invention relates to substituted 1,2,3-triazoles useful for the treatment of ocular diseases, in particular those related to retinal degeneration such as retinitis pigmentosa. The invention also relates to a subgroup of new substituted 1,2,3-triazoles.

### BACKGROUND OF THE INVENTION

The retina is the transparent, light-sensitive structure at the back of the eye. The cornea and lens focus light onto the retina. The retina contains millions of light-sensitive cells (rods and cones) and other nerve cells that receive and organize visual information. The retina sends this information to the brain through the optic nerve, enabling us to see. The central area of the retina, called the macula, contains a high density of color-sensitive photoreceptor (light-sensing) cells. These cells, called cones, produce the sharpest visual images and are responsible for central and color vision. The peripheral area of the retina, which surrounds the macula, contains photoreceptor cells called rods, which respond to lower light levels but are not color sensitive. The rods are responsible for peripheral vision and night vision.

Retinal diseases vary widely, but most of them cause visual symptoms. Some retinal diseases are more common with aging or diabetes. Others are hereditary, such as retinitis pigmentosa, or have genetic risk factors. The inherited diseases of the retina often lead to its degeneration resulting in irreversible loss of vision quite early in life, such as retinitis pigmentosa and Stargardt disease.

Retinitis pigmentosa (RP) is the term used for a genetically heterogenous group of inherited retinal degenerations. Symptoms include night blindness, the development of tunnel vision, and slowly progressive decreased central vision. RP may be: (i) non-syndromic, that is, it occurs alone, without any other clinical findings, (ii) syndromic, with other neurosensory disorders, developmental abnormalities, or complex clinical findings, or (iii) secondary to other systemic diseases. RP combined with deafness (congenital or progressive) is called Usher syndrome. Alport's syndrome is associated with RP and an abnormal glomerular-basement membrane leading to nephrotic syndrome. It is inherited as X-linked dominant.

RP combined with ophthalmoplegia, dysphagia, ataxia, and cardiac conduction defects is seen in the mitochondrial DNA disorder Kearns-Sayre syndrome (also known as Ragged Red Fiber Myopathy). RP combined with retardation, peripheral neuropathy, acanthotic (spiked) RBCs, ataxia, steatorrhea, and absence of VLDL is seen in abetalipoproteinemia. RP is seen clinically in association with several other rare genetic disorders as part of McLeod syndrome. This is an X-linked recessive phenotype characterized by a complete absence of XK cell surface proteins, and therefore markedly reduced expression of all Kell red blood cell antigens. RP associated with hypogonadism, and developmental delay with an autosomal recessive inheritance pattern is seen with Bardet-Biedl syndrome

Some of the major diseases that affect the macula, such as age-related macular degeneration and diabetic retinopathy, lead to impaired vision. Further examples of retinal diseases are cone dystrophies, rod and cone degeneration, Leber congenital amaurosis, retinitis Punctata albescens, choroideremia, choroid and retina gyrate atrophy, generalized choroidal dystrophy, juvenile retinoschisis, Wagner vitreoretinal degeneration, autosomal dominant vitreoretinochoroidopathy, Best vitelliform macular dystrophy, Usher syndrome, Bardet-Biedl syndrome, Sorsby pseudoinflammatory macular dystrophy, and dominant drusen.

There is currently no cure for retinitis pigmentosa, but the efficacy and safety of various prospective treatments are currently being evaluated. WO 2016/146669 A1 discloses the use of cyclic guanosine monophosphate (cGMP) inhibitors. The cGMP inhibitors must be encapsulated in liposomes and daily administered by intraperitoneal injection. ES2673942A1 discloses the use of acylated resveratrol derivatives. The compounds are administered subretinally or complexed with cyclodextrins. Metformin is currently undergoing clinical trials for the treatment of age-related macular degeneration (https://curativebiotech.com/pipeline).

Substituted 1,2,3-triazoles have been described in WO 2017/203083 A1 for the regulation of intracellular calcium homeostasis for use in the prevention or treatment of musculoskeletal, cardiac and neurodegenerative disorders. However, this document is silent about the utility of these compounds in the treatment of ocular diseases.

Substituted 1,2,3-triazoles bearing 4-(aryloxy)methyl groups are suitable to design bioactive compounds, as disclosed by Grimster, et al. J. Am. Chem. Soc., 2012, 134(15), 6732 for nicotinic acetylcholine receptor modulators and Li et al. J. Med. Chem. 2017, 60(7), 2697 for free fatty acid receptor GRP40 agonists. These triazoles have also been used as degradation-free bioligands as disclosed by Hatit et al. Nature Commun. 2018, 9(1), 4021.

Substituted 4-[(arylthio)alkyl]-1H-1,2,3-triazoles have been described in WO 2017/203083 A1 and Aizpurua et al., Eur. J. Med. Chem., 2021, 213 for the regulation of intracellular calcium homeostasis for use in the prevention or treatment of musculoskeletal, cardiac and neurodegenerative disorders. However, these documents are also silent about the utility of these compounds in the treatment of ocular diseases.

There is still a need in the art for adequate treatments of ocular diseases, such as retinal diseases, in particular retinitis pigmentosa and, more particularly, for treatments involving noninvasive administration protocols.

### BRIEF DESCRIPTION OF THE INVENTION

As shown in the examples, the inventors have unexpectedly found that substituted 1,2,3-triazoles are useful for the treatment of ocular diseases, in particular retinal diseases such as retinitis pigmentosa. As also shown in the examples after topical ocular administration, the substituted 1,2,3-triazoles reach the posterior segment of the eye. Thus, these substituted 1,2,3-triazoles can be topically applied and still reach their target site. Topical administration is advantageous since it avoids the use of invasive administration routes, such as intravitreal, and also systemic administration routes, such as oral administration, leading to an improved patient compliance and comfort as well as reduced side effects.

Thus, in the first aspect, the present invention relates to a compound of formula (I):

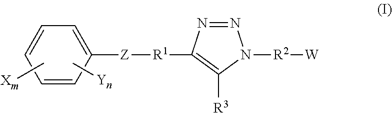

wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, CN, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
R¹ is a linear C₁-C₄ alkylene biradical optionally substituted by one or two substituents independently selected from the group consisting of methyl, ethyl and F;
R² is a linear C₁-C₄ alkylene biradical optionally substituted by one or two substituents independently selected from the group consisting of methyl, ethyl, n-propyl, isopropyl, allyl, progargyl, n-butyl, isobutyl, sec-butyl, tert-butyl, phenyl, 1-naphthyl, 2-naphthyl, benzyl, 4-hydroxybenzyl and CF₃;
R³ is selected from the group consisting of H and I; and
W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof;
for use in the prevention and/or treatment of an ocular disease.

The second aspect relates to a pharmaceutical composition comprising a compound of formula (I) or a pharmaceutically acceptable salt, stereoisomer, tautomer, solvate or N-oxide thereof, or an isotopically labeled derivative thereof as defined in the first aspect and a pharmaceutically acceptable excipient, for use in the prevention and/or treatment of an ocular disease, preferably wherein the pharmaceutically acceptable excipient is selected from the group consisting of hyaluronic acid, carboxymethylcellulose, carbomer and mixtures thereof.

The third aspect relates to a compound of formula (II):

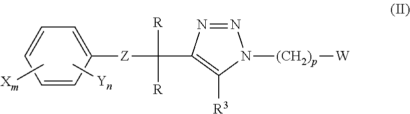

wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
each R is independently selected from the group consisting of H and CH₃;
R³ is selected from the group consisting of H and I;
when Z is —S—, —S(═O)— or —S(═O)₂—, p is selected from the group consisting of 2, 3 and 4, and W is selected from the group consisting of —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH, and —N(C₁-C₄ alkyl)C(═NH)NHC(═NH)NH₂; and
when Z is —O—, p is selected from the group consisting of 2 and 4, and W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof;
with the proviso that the compound is not 1-(2-aminoethyl)-4-[(3,4-methylendioxyphenoxy)methyl]-1H-1,2,3-triazole.

The fourth aspect relates to a compound of formula (II) or a pharmaceutically acceptable salt, stereoisomer, tautomer, solvate or N-oxide thereof, or an isotopically labeled derivative thereof as defined in the third aspect for use in medicine.

The fifth aspect relates to a pharmaceutical composition comprising a compound of formula (II) or a pharmaceutically acceptable salt, stereoisomer, tautomer, solvate or N-oxide thereof, or an isotopically labeled derivative thereof according to the third aspect and a pharmaceutically acceptable excipient; preferably wherein the pharmaceutically acceptable excipient is selected from the group consisting of hyaluronic acid, carboxymethylcellulose, carbomer and mixtures thereof.

The invention also relates to a compound of formula (II) or a pharmaceutically acceptable salt, stereoisomer, tautomer, solvate or N-oxide thereof, or an isotopically labeled derivative thereof according to the third aspect or a pharmaceutical composition according to the fifth aspect for use in the prevention and/or treatment of an ocular disease.

### DESCRIPTION OF THE FIGURES

FIG. 1 shows the biodistribution of the compounds of the invention in 4 mammalian species. Biodistribution of [³H]-35 (whose preparation is described in Example 52) in rat retina was detected using in vivo autoradiography at different time points. Concentration of [³H]-35 reached a peak around 8 h and was detected up to 24 h after administration (A). The bars represent the percentage of administered dose per gram of retina in mouse and rabbit 4 h after single ocular instillation of compound 35, either in phosphate buffered saline (PBS) solution or formulated with 0.2% hyaluronic acid (HA) (B); 6 compounds of the invention formulated with HA attain the swine eye retina in different concentrations (C).

FIG. 2 shows the electroretinographic (ERG) analysis in rd10 mice. Figures show b-wave amplitude (in μV) in untreated rd10 mice (rd10) and treated by daily ocular instillation with compounds 35 (A); 1 (B); 50 (C); 56 (D) and 70 (E) (3 μL of 20 mM solution per eye) for 11 days. Each line represents the mean b-wave in 9 mice for each group+/−standard deviation. To determine differences in group means, 2-tailed Student's test was performed for unpaired samples with comparable variance. *p<0.05; **p<0.005; ***p<0.001 rd10 treated vs. rd10 untreated.

FIG. 3 shows the number of photoreceptor nuclei rows in retinas of rd10 and wild-type mice (WT). Data shown correspond to values obtained from WT mice and from rd10 mice untreated (rd10) or treated with compounds 1, 35, 36 and 53 (3 μL of 20 mM solution per eye) for 11 days. Analysis was performed at central (C), mid-periphery (M-P) and peripheral (P) retina. Bars represent mean values+/−standard deviation (WT control n=3; rd10 untreated n=8; rd10 treated n=4 per treatment). Statistical differences were determined 2-tail Student's test for unpaired samples. *p<0.05 y **p<0.01 rd10 treated vs. rd10 untreated.

FIG. 4 shows morphometric analysis in mouse retina. Representative sections of rd10 (untreated) and rd10 retinas treated with compound 35 labeled with specific markers of rod and cone photoreceptors. A significant preservation of photoreceptors in the retinas of rd10 mice treated for 11 days with compound 35 (3 μL of 20 mM solution per eye) as measured by the number of rods (B) and cones (C) per square mm, or the rod outer and inner segment (D) was observed. Bars represent mean values+/−standard error (rd10 untreated n=3; rd10 treated with compound 35 n=5). To determine differences in group means, 2-tailed Student's test was performed for unpaired samples with comparable variance. **p<0.01 rd10 treated vs. rd10 untreated. Abbreviations: ONL: outer nuclear layer; OS: outer segment; C: central retina; M-P: mid-periphery; P: periphery.

FIG. 5 shows the optomotor test evaluation of visual acuity (contrast sensitivity) of rd10 mice using a modified Morris water maze test. The graph shows contrast sensitivity in untreated rd10 mice (rd10) and treated by daily ocular instillation of compound 35 (3 μL of 20 mM PBS buffered solution per eye) for 16 days. Visual acuity was tested at different contrast and spatial frequencies of the moving visual stimuli. Each dot represents one mouse. For each group+/−standard deviation: *p<0.05; rd10 treated vs. rd10 untreated. The greatest differences were observed in the outlier values of spatial frequency, where there is greater difficulty in recognizing the pattern of moving bars (0.01; 0.02; 0.17 and 0.35 cycles/degree).

FIG. 6 shows in vitro efficacy of compound 35 in preventing phototoxicity of 661 W cells. 661W cells were seeded onto 96-wells MEA impedance plates (50,000 cells/well) and grown until confluence in the Maestro Edge equipment (Axion Biosystems) at 37° C. and 5% CO₂. When impedance reaches a plateau, phototoxicity is induced by addition of 20 μm 9-cis retinal and 30,000 luxes of white light. Increasing concentrations of compound 35 were added (A). Impedance was recorded every minute for 24 hours and was converted to percentage of cytolysis (B).

### DETAILED DESCRIPTION OF THE INVENTION

In the context of the present invention, the following terms have the meaning indicated below:

The term “alkylene biradical” refers to a biradical formed by a linear hydrocarbon chain consisting of carbon and hydrogen atoms, having no unsaturation and bound at its ends to the rest of the molecule through single bonds, such as, for example, methylene, ethylene, propylene, butylene, etc. Mention of a C₁-C₄ alkylene biradical refers to said biradical having between 1 and 4 carbon atoms. The alkylene biradical can be substituted as specified in the definitions of the R₁ and R₂ substituents in the compound of formula (I).

The term “alkyl” refers to a radical formed by a linear or branched hydrocarbon chain consisting of carbon and hydrogen atoms, which does not contain any saturation and is bound to the rest of the molecule by means of a single bond, for example, methyl, ethyl, n-propyl, isopropyl, n-butyl, sec-butyl, tert-butyl, n-pentyl, etc. Mention of a C₁-C₄ alkyl refers to said radical having between 1 and 4 carbon atoms. Mention of a C₁-C₆ alkyl refers to said radical having between 1 and 6 carbon atoms.

The term “halogen” refers to F, Cl, Br and I.

The expression “isotopically labeled derivative” refers to a compound of formula (I) wherein at least one of its atoms is isotopically enriched. For example, compounds of formula (I) in which a hydrogen is replaced with a deuterium or tritium, a carbon is replaced with a ¹³C or ¹⁴C enriched atom, or a nitrogen is replaced with a ¹⁵N enriched atom, are within the scope of this invention. In particular, the isotopically labeled derivatives refer to a compound of formula (I) wherein the hydrogen atom at position R³ is replaced by deuterium or tritium.

The term “pharmaceutically acceptable salts or solvates” refers to any pharmaceutically acceptable salt or solvate, which, when administered to the recipient, is capable of providing (directly or indirectly) a compound of formula (I) as described in the present document. The salts can be prepared by means of methods known in the state of the art.

For example, pharmaceutically acceptable salts of the compounds provided in the present document are synthesized from the previously described basic or acidic unit-containing compound by means of conventional chemical methods. Such salts are generally prepared, for example, by reacting free acid or base forms of these compounds with a stoichiometric amount of the suitable base or acid in water or in an organic solvent or in a mixture of both. Non-aqueous media such as ether, ethyl acetate, ethanol, isopropanol or acetonitrile are generally preferred. Examples of acid addition salts include mineral acid addition salts such as, for example, hydrochloride, hydrobromide, hydroiodide, sulfate, nitrate, phosphate, and organic acid addition salts such as, for example, acetate, maleate, fumarate, citrate, oxalate, succinate, tartrate, malate, mandelate, methanesulfonate and p-toluenesulfonate. Examples of alkaline addition salts include inorganic salts such as, for example, sodium, potassium, calcium, ammonium, magnesium, aluminum and lithium, and organic alkaline salts such as, for example, ethylenediamine, ethanolamine, N,N-dialkylenethanolamine, glucamine and basic amino acid salts.

Solvates refer to a compound of formula (I) in which the molecules of a pharmaceutically suitable solvent are incorporated in the crystal lattice. Solvation methods are generally known in the state of the art. Examples of pharmaceutically suitable solvents are ethanol, water and the like. In a particular embodiment, the solvate is a hydrate.

The compounds of formula (I) or the salts or solvates thereof are preferably in a pharmaceutically acceptable form or a substantially pure form. A pharmaceutically acceptable form is understood, inter alia, as having a pharmaceutically acceptable level of purity, excluding normal pharmaceutical additives such as diluents and excipients, and without including any material considered toxic at normal dosage levels. The levels of purity for the drug are preferably above 50%, more preferably above 70%, and even more preferably above 90%. In a preferred embodiment, it is above 95% of the compound of formula (I) or the salts or solvates thereof.

The compounds of the present invention represented by formula (I) described above can include any stereoisomer depending on the presence of chiral centers, including enantiomers and diastereomers. The individual isomers, enantiomers or diastereomers and mixtures thereof are within the scope of the present invention.

As used herein, the term “tautomer” makes reference to constitutional isomers of the compounds which differ in the position of a proton and a single bond adjacent to a double bond, such as keto-enol tautomers, amide-imide tautomers, amine-imine tautomers, enamine-imine, lactam-lactim tautomer.

The term “N-oxide” refers to derivatives of the compounds of formula (I) wherein a nitrogen atom of an amine groups has been oxidized, i.e. —N⁺O⁻. In particular, N-oxides refer to N-oxides of tertiary amines, preferably to N-oxides of the nitrogen atoms present in the amine substituents defined for W, such as —N(O)(C₁-C₄ alkyl)₂, 1-pyrrolidinyl N-oxide optionally substituted by a C₁-C₄ alkyl, 1-piperidinyl N-oxide optionally substituted by a C₁-C₄ alkyl, 4-morpholinyl N-oxide optionally substituted by a C₁-C₄ alkyl, and 4-piperazinyl N-oxide optionally substituted by a C₁-C₄ alkyl.

The terms “treat”, “treatment”, or “treatment of” as used herein refer to reducing the potential for a certain disease or disorder, reducing the occurrence of a certain disease or disorder, and/or a reduction in the severity of a certain disease or disorder, preferably, to an extent that the subject no longer suffers discomfort and/or altered function due to it. It also refers to mitigating or decreasing at least one clinical symptom and/or inhibition or delay in the progression of the condition and/or prevention or delay of the onset of a disease or illness.

The terms “prevention”, “preventing” or “prevent” as used herein refer to avoiding the appearance of a certain disease or disorder. The prevention can be complete (e.g. the total absence of a disease). The prevention can also be partial, such that for example the occurrence of a disease in a subject is less than that which would have occurred without the administration of the combination or composition of the present invention. Prevention also refers to reduced susceptibility to a clinical condition. The prevention also includes reducing the risk of suffering the disease.

In the first aspect, the present invention relates to a compound of formula (I):

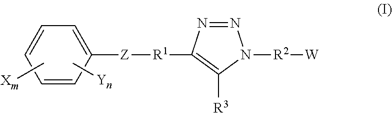

wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, CN, F, Cl, Br and I; or X and
Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
R¹ is a linear C₁-C₄ alkylene biradical optionally substituted by one or two substituents independently selected from the group consisting of methyl, ethyl and F;
R² is a linear C₁-C₄ alkylene biradical optionally substituted by one or two substituents independently selected from the group consisting of methyl, ethyl, n-propyl, isopropyl, allyl, progargyl, n-butyl, isobutyl, sec-butyl, tert-butyl, phenyl, 1-naphthyl, 2-naphthyl, benzyl, 4-hydroxybenzyl and CF₃;
R³ is selected from the group consisting of H and I; and
W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄alkyl), —NHC(═N(C₁-C₄alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof;
for use in the prevention and/or treatment of an ocular disease.

This aspect can also be formulated as the use of a compound of formula (I):

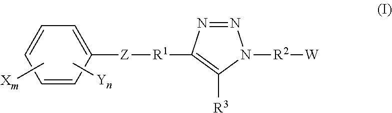

wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, CN, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
R¹ is a linear C₁-C₄ alkylene biradical optionally substituted by one or two substituents independently selected from the group consisting of methyl, ethyl and F;
R² is a linear C₁-C₄ alkylene biradical optionally substituted by one or two substituents independently selected from the group consisting of methyl, ethyl, n-propyl, isopropyl, allyl, progargyl, n-butyl, isobutyl, sec-butyl, tert-butyl, phenyl, 1-naphthyl, 2-naphthyl, benzyl, 4-hydroxybenzyl and CF₃;
R³ is selected from the group consisting of H and I; and
W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof;
in the manufacture of a medicament for the prevention and/or treatment of an ocular disease.

This aspect can also be formulated as a method of prevention and/or treatment of an ocular disease comprising the administration of a compound of formula (I):

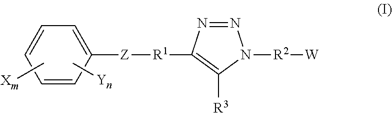

wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, CN, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
R¹ is a linear C₁-C₄ alkylene biradical optionally substituted by one or two substituents independently selected from the group consisting of methyl, ethyl and F;
R² is a linear C₁-C₄ alkylene biradical optionally substituted by one or two substituents independently selected from the group consisting of methyl, ethyl, n-propyl, isopropyl, allyl, progargyl, n-butyl, isobutyl, sec-butyl, tert-butyl, phenyl, 1-naphthyl, 2-naphthyl, benzyl, 4-hydroxybenzyl and CF₃;
R³ is selected from the group consisting of H and I; and
W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof.

The X substituent is present when m has a value other than 0. Similarly, the Y substituent is present when n has a value other than 0.

Preferably, in the compound of formula (I) for use according to the invention, m is selected from the group consisting of 0, 1 and 2, more preferably m is 1.

Preferably, in the compound of formula (I) for use according to the invention, n is 0 or 1, more preferably n is 0.

Preferably, in the compound of formula (I) for use according to the invention, m is 1; n is 0; and X is in para position with respect to Z.

Preferably, in the compound of formula (I) for use according to the invention, X and Y (when present, i.e. wherein m and/or n are other than 0) are independently selected from the group consisting of C₁-C₆ alkyl, OH, O(C₁-C₄ alkyl), CF₃, CN, F, Cl, Br and I; or X and Y together represent a methylenedioxy or ethylenedioxy biradical. More preferably, in the compound of formula (I) for use according to the invention, X and Y (when present, i.e. wherein m and/or n are other than 0) are independently selected from the group consisting of methyl, isopropyl, OH, methoxy, CF₃, and Br; or X and Y together represent a methylenedioxy biradical. Even more preferably, X is methoxy. Still more preferably X is methoxy, m is 1, and Y is absent (i.e. n is 0).

Preferably, in the compound of formula (I) for use according to the invention, X (when present, i.e. wherein m is other than 0) is independently selected from the group consisting of C₁-C₆ alkyl, OH, O(C₁-C₄ alkyl), CF₃, CN, F, Cl, Br and I; or X and Y together represent a methylenedioxy or ethylenedioxy biradical. More preferably, in the compound of formula (I) for use according to the invention, X (when present, i.e. wherein m is other than 0) is independently selected from the group consisting of methyl, isopropyl, OH, methoxy, CF₃, and Br; or X and Y together represent a methylenedioxy biradical. Even more preferably, X is methoxy. Still more preferably X is methoxy and m is 1.

Preferably, in the compound of formula (I) for use according to the invention, Y (when present, i.e. wherein n is other than 0) is independently selected from the group consisting of C₁-C₆ alkyl and O(C₁-C₄ alkyl); or X and Y together represent a methylenedioxy or ethylenedioxy biradical. More preferably, in the compound of formula (I) for use according to the invention, Y (when present, i.e. wherein n is other than 0) is independently selected from the group consisting of methyl and methoxy; or X and Y together represent a methylenedioxy biradical. Even more preferably, Y is absent (i.e. n is 0).

Preferably, in the compound of formula (I) for use according to the invention, X (when present, i.e. wherein m is other than 0) is independently selected from the group consisting of C₁-C₆ alkyl, OH, O(C₁-C₄ alkyl), CF₃, CN, F, Cl, Br and I; Y (when present, i.e. wherein n is other than 0) is independently selected from the group consisting of C₁-C₆ alkyl and O(C₁-C₄ alkyl); or X and Y together represent a methylenedioxy or ethylenedioxy biradical. More preferably, in the compound of formula (I) for use according to the invention, X (when present, i.e. wherein m is other than 0) is independently selected from the group consisting of methyl, isopropyl, OH, methoxy, CF₃, and Br; Y (when present, i.e. wherein n is other than 0) is independently selected from the group consisting of methyl and methoxy; or X and Y together represent a methylenedioxy biradical. Even more preferably, X is methoxy. Still more preferably X is methoxy, m is 1, and Y is absent (i.e. n is 0).

Preferably, in the compound of formula (I) for use according to the invention, Z is selected from the group consisting of —S— and —O—, more preferably, Z is —S—.

Preferably, in the compound of formula (I) for use according to the invention, R¹ is a linear C₁-C₄ alkylene biradical optionally substituted by one or two substituents independently selected from the group consisting of methyl and ethyl. More preferably, R¹ is a C₁-C₂ alkylene biradical optionally substituted by two methyl substituents. Even more preferably, R¹ is selected from the group consisting of —CH₂— and —C(CH₃)₂—. Still more preferably, R¹ is —CH₂—

Preferably, in the compound of formula (I) for use according to the invention, R² is a linear C₁-C₄ alkylene biradical optionally substituted by one or two substituents independently selected from the group consisting of methyl and benzyl. More preferably, R² is a linear C₂-C₃ alkylene biradical optionally substituted by one or two substituents independently selected from the group consisting of methyl and benzyl. Even more preferably, R² is selected from the group consisting of —CH₂—CH₂—, —CH(Bn)—CH₂—, —CH₂—CH₂—CH₂— and —C(CH₃)₂—CH₂—. Still more preferably, R² is selected from the group consisting of —CH₂—CH₂— and —CH₂—CH₂—CH₂—. Still more preferably, R² is —CH₂—CH₂—.

Preferably, in the compound of formula (I) for use according to the invention, R³ is H.

Preferably, in the compound of formula (I) for use according to the invention, W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, and —N(C₁-C₄ alkyl)C(═NH)NHC(═NH)NH₂. More preferably, W is selected from the group consisting of —NH₂, —NH(C₁-C₄ alkyl), —N(C₁-C₄ alkyl)₂, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, —NHC(═NH)NH₂, and —NHC(═NH)NHC(═NH)NH₂. Even more preferably, W is selected from the group consisting of —NH₂, —NH(CH₃), —N(CH₃)₂, —N(CH₂CH₃)₂, 1-pyrrolidinyl, 1-piperidinyl, 4-morpholinyl, —NHC(═NH)NH₂, and —NHC(═NH)NHC(═NH)NH₂. Still more preferably, W is selected from the group consisting of —N(CH₃)₂, 1-piperidinyl, 4-morpholinyl, and NHC(═NH)NHC(═NH)NH₂. Still more preferably, W is —N(CH₃)₂.

In one embodiment, in the compound of formula (I) for use according to the invention, m is 1; n is 0; X is selected from the group consisting of O(C₁-C₄ alkyl), C₁-C₆ alkyl and CF₃; R¹ is —CH₂—; R² is selected from the group consisting of —CH₂—CH₂— and —CH₂—CH₂—CH₂—; R³ is H; and W is selected from the group consisting of —N(C₁-C₄alkyl)₂, 1-piperidinyl, 4-morpholinyl, 1-pyrrolidinyl, 4-piperazinyl optionally substituted by a C₁-C₄ alkyl, and NHC(═NH)NHC(═NH)NH₂.

In one embodiment, in the compound of formula (I) for use according to the invention, m is 1; n is 0; X is selected from the group consisting of methoxy, isopropyl and CF₃; R¹ is —CH₂—; R² is selected from the group consisting of —CH₂—CH₂— and —CH₂—CH₂—CH₂—; R³ is H; and W is selected from the group consisting of —N(CH₃)₂, 1-piperidinyl, 4-morpholinyl, and NHC(═NH)NHC(═NH)NH₂.

In one embodiment, in the compound of formula (I) for use according to the invention, Z is O or S; m is 1; n is 0; X is methoxy; R¹ is —CH₂—; R² is —CH₂—CH₂—; R³ is H; and W is —N(CH₃)₂.

In one embodiment, in the compound of formula (I) for use according to the invention, Z is O or S; m is 1; n is 0; X is methoxy; R¹ is —CH₂—; R² is —CH₂—CH₂—; R³ is H; W is —N(CH₃)₂; and X is in para position with respect to Z.

In another embodiment, in the compound of formula (I) for use according to the invention, Z is —S—; R¹ is —CH₂—; and R³ is H.

In a preferred embodiment, the compound of formula (I) for use according to the invention, is selected from the group consisting of:
1-[2-(N,N-dimethylamino)ethyl]-4-[(4-methoxyphenoxy)methyl]-1H-1,2,3-triazole;
1-[2-(N,N-dimethylamino)ethyl]-4-[(3,4-methylendioxyphenoxy)methyl]-1H-1,2,3-triazole;
1-[2-(N,N-dimethylamino)ethyl]-4-[(3-methoxyphenoxy)methyl]-1H-1,2,3-triazole;
4-[(3,5-dimethoxyphenoxy)methyl]-1-[2-(N,N-dimethylamino)ethyl]-1H-1,2,3-triazole;
1-[2-(N,N-dimethylamino)ethyl]-4-(phenoxymethyl)-1H-1,2,3-triazole;
1-[2-(N,N-dimethylamino)ethyl]-4-[(3-trifluoromethylphenoxy)methyl]-1H-1,2,3-triazole;
1-[2-(N,N-dimethylamino)ethyl]-4-[(3,4,5-trimethoxyphenoxy)methyl]-1H-1,2,3-triazole;
4-[(3,4-dimethoxyphenoxy)methyl]-1-[2-(N,N-dimethylamino)ethyl]-1H-1,2,3-triazole;
1-[2-(N,N-dimethylamino)ethyl]-4-[1-(4-methoxyphenoxy)-1-(methyl)-ethyl]-1H-1,2,3-triazole;
4-[(4-methoxyphenoxy)methyl]-1-[2-(N-methylamino)ethyl]-1H-1,2,3-triazole;
1-(2-aminoethyl)-4-[(4-methoxyphenoxy)methyl]-1H-1,2,3-triazole;
1-(2-aminoethyl)-4-[(3-methoxyphenoxy)methyl]-1H-1,2,3-triazole;
1-(2-aminoethyl)-4-[(3,4-methylendioxyphenoxy)methyl]-1H-1,2,3-triazole;
1-(2-aminoethyl)-4-(phenoxymethyl)-1H-1,2,3-triazole;
1-(2-aminoethyl)-4-[1-(4-methoxyphenoxy)-1-(methyl)-ethyl]-1H-1,2,3-triazole;
4-[(4-methoxyphenoxy)methyl]-1-[2-(pyrrolidin-1-yl)ethyl]-1H-1,2,3-triazole;
4-[(4-methoxyphenoxy)methyl]-1-[2-(morpholin-4-yl)ethyl]-1H,1,2,3-triazole;
4-[(4-methoxyphenoxy)methyl]-1-[2-(piperidin-1-yl)ethyl]-1H-1,2,3-triazole;
1-[2-(piperidin-1-yl)ethyl]-4-[(3-trifluoromethylphenoxy)methyl]-1H-1,2,3-triazole;
1-[2-(piperidin-1-yl)ethyl]-4-[(4-trifluoromethylphenoxy)methyl]-1H-1,2,3-triazole;
1-[2-(piperidin-1-yl)ethyl]-4-[(2-trifluoromethylphenoxy)methyl]-1H-1,2,3-triazole;
(guanidyl)ethyl]-4-[(4-methoxyphenoxy)methyl]-1H-1,2,3-triazole;
1-[2-(guanidyl)ethyl]-4-[(3-methoxyphenoxy)methyl]-1H-1,2,3-triazole;
1-[2-(guanidyl)ethyl]-4-[(3,4-methylendioxyphenoxy)methyl]-1H-1,2,3-triazole;
1-[2-(guanidyl)ethyl]-4-(phenoxymethyl)-1H-1,2,3-triazole;
1-[2-(guanidyl)ethyl]-4-[1-(4-methoxyphenoxy)-1-(methyl)-ethyl]-1H-1,2,3-triazole;
1-[2-(biguanidyl)ethyl]-4-[(4-methoxyphenoxy)methyl]-1H-1,2,3-triazole;
1-[2-(biguanidyl)ethyl]-4-[(3-methoxyphenoxy)methyl]-1H-1,2,3-triazole;
1-[2-(biguanidyl)ethyl]-4-[(3,4-methylendioxyphenoxy)methyl]-1H-1,2,3-triazole;
1-[2-(biguanidyl)ethyl]-4-(phenoxymethyl)-1H-1,2,3-triazole;
1-[2-(biguanidyl)ethyl]-4-[(4-methoxyphenoxy)-1-(methyl)-ethyl]-1H-1,2,3-triazole;
1-[2-(N,N-dimethylamino)ethyl]-4-[(4-hydroxyphenoxy)methyl]-1H-1,2,3-triazole;
5-iodo-4-[(4-methoxyphenoxy)methyl]-1-[2-(N,N-dimethylamino)ethyl]-1H-1,2,3-triazole;
1-[2-(N,N-dimethyl)-N-oxideaminoethyl]-4-[(4-methoxyphenoxy)methyl]-1H-1,2,3-triazole;
1-[2-(N,N-dimethylamino)ethyl]-4-[(4-methoxyphenyl)thiomethyl]-1H-1,2,3-triazole;
1-[3-(N,N-dimethylamino)propyl]-4-[(4-methoxyphenyl)thiomethyl]-1H-1,2,3-triazole;
1-[2-(N,N-diethylamino)ethyl]-4-[(4-methoxyphenyl)thiomethyl]-1H-1,2,3-triazole;
1-[2-(piperidin-1-yl)ethyl]-4-[(3-(trifluoromethylphenyl)thiomethyl]-1H-1,2,3-triazole;
4-[(4-methoxyphenyl)thiomethyl]-1-[2-(piperidin-1-yl)ethyl]-1H-1,2,3-triazole;
4-[(4-tert-butylphenyl)thiomethyl]-1-[2-(piperidin-1-yl)ethyl]-1H-1,2,3-triazole;
4-[(4-isopropylphenyl)thiomethyl]-1-[2-(4-morpholinyl)ethyl]-1H,1,2,3-triazole;
1-[2-(4-morpholinyl)ethyl]-4-(phenylthiomethyl)-1H,1,2,3-triazole;
1-[2-(N,N-dimethylamino)ethyl]-4-[(4-hydroxyphenyl)thiomethyl]-1H-1,2,3-triazole;
1-[1-(R)-benzyl-2-(piperidin-1-yl)ethyl]-4-[(4-methoxyphenyl)thiomethyl]-1H-1,2,3-triazole;
1-[1-(R)-benzyl-2-(piperidin-1-yl)ethyl]-4-[(3-trifluoromethylphenyl)thiomethyl]-1H-1,2,3-triazole;
1-[1-(R)-benzyl-2-(piperidin-1-yl)ethyl]-4-[(4-tert-butylphenyl)thiomethyl]-1H-1,2,3-triazole;
1-[3-(N,N-diethylamino)-2-methyl-propyl]-4-[(4-methoxyphenyl)thiomethyl]-1H-1,2,3-triazole;
1-[2-(guanidyl)ethyl]-4-[(4-methoxyphenyl)thiomethyl]-1H-1,2,3-triazole;
1-[2-(biguanidinyl)ethyl]-4-[(4-methoxyphenyl)thiomethyl]-1H-1,2,3-triazole;
1-[3-(biguanidinyl)propyl]-4-[(4-methoxyphenyl)thiomethyl]-1H-1,2,3-triazole;
1-[2-(N,N-dimethylamino)ethyl]-5-iodo-4-[(4-methoxyphenyl)thiomethyl]-1H-1,2,3-triazole;
1-[2-(N,N-dimethylamino)ethyl]-5-trithium-4-[(4-methoxyphenyl)thiomethyl]-1H-1,2,3-triazole;
1-[2-(N,N-dimethylamino)ethyl]-4-[(4-methoxyphenyl)sulfinylmethyl]-1H-1,2,3-triazole;
1-[3-(N,N-dimethylamino)propyl]-4-[(4-methoxyphenyl)sulfinylmethyl]-1H-1,2,3-triazole;
1-[2-(N,N-diethylamino)ethyl]-4-[(4-methoxyphenyl)sulfinylmethyl]-1H-1,2,3-triazole;
1-[2-(piperidin-1-yl)ethyl]-4-[(3-trifluoromethylphenyl)sulfinylmethyl]-1H-1,2,3-triazole;
4-[(4-isopropylphenyl)sulfinylmethyl]-1-[2-(4-morpholinyl)ethyl]-1H,1,2,3-triazole;
4-[(2,6-dimethylphenyl)sulfinylmethyl]-1-[2-(piperidin-1-yl)ethyl]-1H-1,2,3-triazole;
1-[2-(4-morpholinyl)ethyl]-4-(phenylsulfinylmethyl)-1H,1,2,3-triazole;
4-[(4-methoxyphenyl)sulfinylmethyl]-1-[2-(piperidin-1-yl)ethyl]-1H-1,2,3-triazole;
1-[1-(R)-benzyl-2-(piperidin-1-yl)ethyl]-4-[(3-trifluoromethylphenyl)sulfinylmethyl]-1H-1,2,3-triazole;
1-[3-(N,N-diethylamino)-2-methyl-propyl]-4-[(4-methoxyphenyl)sulfinylmethyl]-1H-1,2,3-triazole;
1-[2-(N,N-dimethylamino)ethyl]-4-[(4-hydroxyphenyl)sulfinylmethyl]-1H-1,2,3-triazole;
1-(2-aminoethyl)-4-[(4-methoxyphenyl)sulfinylmethyl]-1H-1,2,3-triazole;
1-[2-(N-methylamino)-ethyl]-4-[(4-methoxyphenyl)sulfinylmethyl]-1H-1,2,3-triazole;
1-[2-(N,N-dimethyl)-N-oxideaminoethyl]-4-[(4-methoxyphenyl)sulfinylmethyl]-1H-1,2,3-triazole;
1-[2-(N,N-dimethylamino)ethyl]-4-[(4-methoxyphenyl)sulfonylmethyl]-1H-1,2,3-triazole;
1-[2-(N,N-diethylamino)ethyl]-4-[(4-bromophenyl)sulfonylmethyl]-1H-1,2,3-triazole;
1-[2-(N,N-dimethylamino)ethyl]-4-[(3,4-dimethoxyphenyl)sulfonylmethyl]-1H-1,2,3-triazole;
4-[(4-isopropylphenyl)sulfonylmethyl]-1-[2-(4-morpholinyl)ethyl]-1H,1,2,3-triazole;
4-(phenylsulfonylmethyl)-1-[2-(4-morpholinyl)ethyl]-1H,1,2,3-triazole;
1-[2-(N,N-dimethylamino)ethyl]-4-[(3-trifluoromethylphenyl)sulfonylmethyl]-1H-1,2,3-triazole;
1-(2-aminoethyl)-4-[(4-methoxyphenyl)sulfonylmethyl]-1H-1,2,3-triazole; and
1-[2-(N-methylamino)-ethyl]-4-[(4-methoxyphenyl)sulfonylmethyl]-1H-1,2,3-triazole.

Even more preferably, the compound of formula (I) for use according to the invention is selected from the group consisting of 1-[2-(N,N-dimethylamino)ethyl]-4-[(4 -methoxyphenoxy)methyl]-1H-1,2,3-triazole, 1-[2-(N,N-dimethylamino)ethyl]-4-[(4-methoxyphenyl)thiomethyl]-1H-1,2,3-triazole, 1-[2-(guanidyl)ethyl]-4-[(3,4-methylendioxyphenoxy)methyl]-1H-1,2,3-triazole and 1-[2-(biguanidyl)ethyl]-4-[(4-methoxyphenyl)thiomethyl]-1H-1,2,3-triazole.

Still more preferably, the compound of formula (I) for use according to the invention is 1-[2-(N,N-dimethylamino)ethyl]-4-[(4-methoxyphenyl)thiomethyl]-1H-1,2,3-triazole.

In a preferred embodiment, the ocular disease is a retinal disease. Preferably, the retinal disease is selected from the group consisting of retinitis pigmentosa (such as autosomal dominant retinitis pigmentosa, autosomal recessive retinitis pigmentosa, X-linked retinitis pigmentosa, sporadic retinitis pigmentosa, retinitis pigmentosa associated with other syndromes), diabetic retinopathy, cone dystrophies, rod and cone degeneration, Leber congenital amaurosis, retinitis Punctata albescens, choroideremia, choroid and retina gyrate atrophy, generalized choroidal dystrophy, juvenile retinoschisis, Wagner vitreoretinal degeneration, autosomal dominant vitreoretinochoroidopathy, Stargardt disease, Best vitelliform macular dystrophy, Usher syndrome, Bardet-Biedl syndrome, Sorsby pseudoinflammatory macular dystrophy, age-related macular degeneration, autosomal dominant congenital stationary night blindness, achromatopsia and dominant drusen. More preferably, the ocular disease is selected from the group consisting of retinitis pigmentosa (such as autosomal dominant retinitis pigmentosa, autosomal recessive retinitis pigmentosa, X-linked retinitis pigmentosa, sporadic retinitis pigmentosa, retinitis pigmentosa associated with other syndromes), Stargardt disease, Leber congenital amaurosis, age-related macular degeneration and diabetic retinopathy.

Still more preferably, the ocular disease is retinitis pigmentosa (such as autosomal dominant retinitis pigmentosa, autosomal recessive retinitis pigmentosa, X-linked retinitis pigmentosa, sporadic retinitis pigmentosa, retinitis pigmentosa associated with other syndromes).

In another preferred embodiment, the retinal disease is selected from retinitis pigmentosa, autosomal dominant congenital stationary night blindness and achromatopsia.

The subject to which the compounds and pharmaceutical compositions described herein are administered is a human or animal; preferably subjects are mammals, more preferably humans.

For its administration to a subject, such as a mammal, e.g., a human, in need of treatment, the compounds and pharmaceutical composition described herein may be administered by any appropriate route, such as, topical, intraocular, oral (e.g., oral, sublingual, etc.), parenteral (e.g., subcutaneous, intramuscular, intravenous, intramuscular, etc.), rectal, nasal, etc. Thus, in one embodiment, the compounds and pharmaceutical compositions described herein are administered topically, intraocularly (e.g. subretinally or intravitreally), orally (including sublingual administration), parenterally (e.g. intravenously, intramuscularly, intraperitoneally, subcutaneously), transdermally, intranasally, or rectally. In a preferred embodiment, the compounds and pharmaceutical composition as defined herein are administered topically on the corneal surface, preferably in the form of drops, gel, cream, spray, contact lenses or intraocular rings.

The pharmaceutical compositions comprise a compound as described herein and one or more pharmaceutically acceptable excipients or vehicles.

The pharmaceutically acceptable vehicle must be acceptable in the sense of being compatible with other ingredients of the composition and not being harmful to the receiver thereof. Said pharmaceutically acceptable vehicle can be selected from organic and inorganic materials that are used in pharmaceutical formulations and incorporated as analgesic agents, pH regulators, linkers, disintegrants, diluents, emulsifiers, fillers, glidants, solubilizers, stabilizers, suspension agents, tonicity agents and thickeners. Pharmaceutical additives such as antioxidants, aromatic agents, dyes, aroma enhancing agents, preservatives and sweeteners can furthermore be added.

The compounds and pharmaceutical composition described herein may be administered in the form of different preparations. Examples are preparations for oral administration, i.e. tablets, capsules, syrups or suspensions. Also, the pharmaceutical compositions of the invention may include topical compositions, i.e. creams, ointments or pastes, or transdermic preparations such as patches or plasters. The pharmaceutical composition of the invention may also be prepared for rectal administration, i.e. rectal gel or rectal capsule.

Suitable dose forms for oral administration may be tablets, capsules, syrups or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tableting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

In a preferred embodiment, the pharmaceutical compositions comprise a pharmaceutically acceptable excipient selected from the group consisting of hyaluronic acid, carboxymethylcellulose, carbomer and mixtures thereof.

Even more preferably, the pharmaceutical composition comprises a compound of formula (I) selected from the group consisting of 1-[2-(N,N-dimethylamino) ethyl]-4-[(4-methoxyphenoxy)methyl]-1H-1,2,3-triazole, 1-[2-(N,N-dimethylamino)ethyl]-4-[(4-methoxyphenyl)thiomethyl]-1H-1,2,3-triazole, 1-[2-(guanidyl)ethyl]-4-[(3,4-methylendioxyphenoxy)methyl]-1H-1,2,3-triazole and 1-[2-(biguanidyl)ethyl]-4-[(4-methoxyphenyl)thiomethyl]-1H-1,2,3-triazole, and a pharmaceutically acceptable excipient selected from the group consisting of hyaluronic acid, carboxymethylcellulose, carbomer and mixtures thereof.

Still more preferably, the pharmaceutical composition comprises 1-[2-(N,N-dimethylamino)ethyl]-4-[(4-methoxyphenyl)thiomethyl]-1H-1,2,3-triazole and hyaluronic acid.

By an “effective” amount or a “therapeutically effective amount” of a compound is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount”. However, an appropriate “effective” amount in any individual case may be determined by one of ordinary skill in the art using routine experimentation. The compounds will typically be administered once or more times a day, for example 1, 2, 3 or 4 times daily, with typical total daily doses in the range of from 0.001 to 500 mg/day, preferably at a dose of 0.001 to 10 mg/day.

In another aspect, the invention relates to a compound of formula (II), which is a subgroup of the compound of formula (I):

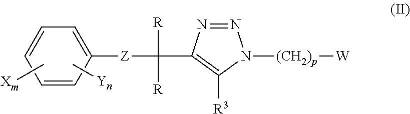

wherein
X and Y are independently selected from the group consisting of C₁-C₆ alkyl, OH, O(C₁-C₄alkyl), OCF₃, S(C₁-C₄ alkyl), NHC(O)(C₁-C₄ alkyl), CF₃, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
each R is independently selected from the group consisting of H and CH₃;
R³ is selected from the group consisting of H and I;
when Z is —S—, —S(═O)— or —S(═O)₂—, p is selected from the group consisting of 1, 2, 3 and 4, and W is selected from the group consisting of —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, and —N(C₁-C₄ alkyl)C(═NH)NHC(═NH)NH₂; and
when Z is —O—, p is selected from the group consisting of 1, 2 and 4, and W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof;
with the proviso that the compound is not 1-(2-aminoethyl)-4-[(3,4-methylendioxyphenoxy)methyl]-1H-1,2,3-triazole.

The X substituent is present when m has a value other than 0. Similarly, the Y substituent is present when n has a value other than 0.

In a preferred embodiment, in the compound of formula (II), when Z is —O—,
m is selected from the group consisting of 0, 1, 2 and 3;
n is selected from the group consisting of 0, 1 and 2;
X, when present, is in meta or para position with respect to Z and is selected from the group consisting of OH, O(C₁-C₄ alkyl), OCF₃ and CF₃;
Y, when present, is in ortho position with respect to Z and is CF₃;
or X and Y together represent a methylenedioxy or ethylenedioxy biradical;
each R is independently selected from the group consisting of H and CH₃;
R³ is selected from the group consisting of H and I;
p is selected from the group consisting of 2 and 4;
W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), NHC(═NH)NHC(═NH)NH₂, and —N(C₁-C₄ alkyl)C(═NH)NHC(═NH)NH₂.

In the above embodiment, when Z is —S—, —S(═O)— or —S(═O)₂—,
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, F, Cl, Br and I; or X and Y together represent a methylenedioxy or ethylenedioxy biradical;
m and n are independently selected from the group consisting of 0, 1, 2, 3 and 4;
each R is independently selected from the group consisting of H and CH₃;
R³ is selected from the group consisting of H and I;
p is selected from the group consisting of 2, 3 and 4;
W is selected from the group consisting of —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, and —N(C₁-C₄ alkyl)C(═NH)NHC(═NH)NH₂.

In a preferred embodiment of the compound of formula (II) m is 1; n is 0; X is in para position with respect to Z; X is O(C₁-C₄ alkyl); Z is —S— or —O—; R is H; p is 2; R³ is H; when Z is —S— W is NHC(═NH)NHC(═NH)NH₂; and when Z is —O— W is —N(C₁-C₄ alkyl)₂.

In a preferred embodiment of the compound of formula (II) m is 1; n is 0; X is in para position with respect to Z; X is O(C₁-C₄ alkyl); Z is —O—; R is H; p is 2; R³ is H; and W is —N(C₁-C₄ alkyl)₂.

The X substituent is present when m has a value other than 0. Similarly, the Y substituent is present when n has a value other than 0.

Preferably, in the compound of formula (II) according to the invention, m is selected from the group consisting of 0, 1 and 2, more preferably m is 1.

Preferably, in the compound of formula (II) according to the invention, n is 0 or 1, more preferably n is 0.

Preferably, in the compound of formula (II) according to the invention, m is 1; n is 0; and X is in para position with respect to Z.

Preferably, in the compound of formula (II) according to the invention, X and Y (when present, i.e. wherein m and/or n are other than 0) are independently selected from the group consisting of O(C₁-C₄ alkyl), OH and CF₃; or X and Y together represent a methylenedioxy or ethylenedioxy biradical. More preferably, in the compound of formula (II) according to the invention, X and Y (when present, i.e. wherein m and/or n are other than 0) are independently selected from the group consisting of methoxy, OH and CF₃; or X and Y together represent a methylenedioxy biradical. Even more preferably, X is methoxy. Still more preferably X is methoxy, m is 1, and Y is absent (i.e. n is 0).

In a particular embodiment, X, when present, is in meta or para position with respect to Z and is selected from the group consisting of O(C₁-C₄ alkyl), OH, and CF₃; and Y, when present, is in ortho position with respect to Z and is CF₃.

Preferably, in the compound of formula (II) according to the invention, X (when present, i.e. wherein m is other than 0) is independently selected from the group consisting of O(C₁-C₄ alkyl), OH and CF₃; or X and Y together represent a methylenedioxy or ethylenedioxy biradical. More preferably, in the compound of formula (II) according to the invention, X (when present, i.e. wherein m is other than 0) is independently selected from the group consisting of methoxy, OH and CF₃; or X and Y together represent a methylenedioxy biradical. Even more preferably, X is methoxy. Still more preferably X is methoxy and m is 1.

Preferably, in the compound of formula (II) for use according to the invention, Y (when present, i.e. wherein n is other than 0) is independently a O(C₁-C₄ alkyl); or X and Y together represent a methylenedioxy or ethylenedioxy biradical. More preferably, in the compound of formula (II) according to the invention, Y (when present, i.e. wherein n is other than 0) is independently methoxy; or X and Y together represent a methylenedioxy biradical. Even more preferably, Y is absent (i.e. n is 0).

Preferably, in the compound of formula (II) according to the invention, X (when present, i.e. wherein m is other than 0) is independently selected from the group consisting O(C₁-C₄ alkyl), OH and CF₃; Y (when present, i.e. wherein n is other than 0) is independently a O(C₁-C₄ alkyl); or X and Y together represent a methylenedioxy or ethylenedioxy biradical. More preferably, in the compound of formula (II) according to the invention, X (when present, i.e. wherein m is other than 0) is independently selected from the group consisting of methoxy, OH and CF₃; Y (when present, i.e. wherein n is other than 0) is independently a methoxy; or X and Y together represent a methylenedioxy biradical.

Even more preferably, X is methoxy. Still more preferably X is methoxy, m is 1, and Y is absent (i.e. n is 0).

Preferably, in the compound of formula (II) according to the invention, X (when present, i.e. wherein m is 0) is in meta or para position with respect to Z, even more preferably in para position.

Preferably, in the compound of formula (II) according to the invention, Y (when present, i.e. wherein n is 0) is in ortho position with respect to Z.

Preferably, in the compound of formula (II) according to the invention, Z is selected from the group consisting of —S— and —O—. In one embodiment, Z is —S—. In another embodiment, Z is —O—.

Preferably, in the compound of formula (II) according to the invention, each R is independently selected from H and CH₃; even more preferably, R is H.

Preferably, in the compound of formula (II) according to the invention, p is selected from 2 and 4. More preferably, p is 2.

Preferably, in the compound of formula (II) according to the invention, R³ is H.

Preferably, in the compound of formula (II) according to the invention, when Z is —S—, —S(═O)— or —S(═O)₂—, W is selected from the group consisting of —NHC(═NH)NH₂ and —NHC(═NH)NHC(═NH)NH₂. Still more preferably, when Z is —S—, S(═O)— or —S(═O)₂—, W is-NHC(═NH)NHC(═NH)NH₂.

Preferably, in the compound of formula (II) according to the invention, when Z is —O—, W is selected from the group consisting —NH₂, —NH(C₁-C₄ alkyl), —N(C₁-C₄ alkyl)₂, 1-pyrrolidinyl optionally substituted by a C₁-C₄ alkyl, 1-piperidinyl optionally substituted by a C₁-C₄ alkyl, and 4-morpholinyl optionally substituted by a C₁-C₄ alkyl. Even more preferably, when Z is —O—, W is selected from the group consisting of —NH₂, —NH(CH₃), —N(CH₃)₂, 1-pyrrolidinyl, 1-piperidinyl, and 4-morpholinyl. Still more preferably, when Z is —O—, W is —N(CH₃)₂.

In a particular embodiment, in the compound of formula (II) according to the invention, m is 1; n is 0; X is selected from the group consisting of O(C₁-C₄ alkyl), OH and CF₃; R is H; p is 2; R³ is H; Z is —O— or —S—, when Z is —O—, W is selected from the group consisting of —N(C₁-C₄ alkyl)₂, 1-piperidinyl, 4-morpholinyl, 1-pyrrolidinyl, optionally substituted by a C₁-C₄ alkyl, and when Z is —S— W is NHC(═NH)NHC(═NH)NH₂.

In a particular embodiment, in the compound of formula (II) according to the invention, m is 1; n is 0; X is selected from the group consisting of methoxy, OH and CF₃; R is H; p is 2; R³ is H; and Z is —O— or —S—, and when Z is —O—, W is selected from the group consisting of —N(C₁-C₄ alkyl)₂, 1-piperidinyl, 4-morpholinyl, 1-pyrrolidinyl, and when Z is —S— W is NHC(═NH)NHC(═NH)NH₂.

In another particular embodiment, in the compound of formula (II) according to the invention, Z is —O—; m is 1; n is 0; X is methoxy; R is H; p is 2; R³ is H; and W is —N(CH₃)₂.

In another particular embodiment, in the compound of formula (II) according to the invention, Z is —O—; m is 1; n is 0; X is methoxy; R is H; p is 2; R³ is H; W is —N(CH₃)₂; and X is in para position with respect to Z.

In a preferred embodiment, the compound of formula (II) is selected from the group consisting of:
1-[2-(N,N-dimethylamino)ethyl]-4-[(4-methoxyphenoxy)methyl]-1H-1,2,3-triazole;
1-[2-(N,N-dimethylamino)ethyl]-4-[(3,4-methylendioxyphenoxy)methyl]-1H-1,2,3-triazole;
1-[2-(N,N-dimethylamino)ethyl]-4-[(3-methoxyphenoxy)methyl]-1H-1,2,3-triazole;
4-[(3,5-dimethoxyphenoxy)methyl]-1-[2-(N,N-dimethylamino)ethyl]-1H-1,2,3-triazole;
1-[2-(N,N-dimethylamino)ethyl]-4-(phenoxymethyl)-1H-1,2,3-triazole;
1-[2-(N,N-dimethylamino)ethyl]-4-[(3-trifluoromethylphenoxy)methyl]-1H-1,2,3-triazole;
1-[2-(N,N-dimethylamino)ethyl]-4-[(3,4,5-trimethoxyphenoxy)methyl]-1H-1,2,3-triazole;
4-[(3,4-dimethoxyphenoxy)methyl]-1-[2-(N,N-dimethylamino)ethyl]-1H-1,2,3-triazole;
1-[2-(N,N-dimethylamino)ethyl]-4-[1-(4-methoxyphenoxy)-1-(methyl)-ethyl]-1H-1,2,3-triazole;
4-[(4-methoxyphenoxy)methyl]-1-[2-(N-methylamino)ethyl]-1H-1,2,3-triazole;
1-(2-aminoethyl)-4-[(4-methoxyphenoxy)methyl]-1H-1,2,3-triazole;
1-(2-aminoethyl)-4-[(3-methoxyphenoxy)methyl]-1H-1,2,3-triazole;
1-(2-aminoethyl)-4-[(3,4-methylendioxyphenoxy)methyl]-1H-1,2,3-triazole;
1-(2-aminoethyl)-4-(phenoxymethyl)-1H-1,2,3-triazole;
1-(2-aminoethyl)-4-[1-(4-methoxyphenoxy)-1-(methyl)-ethyl]-1H-1,2,3-triazole;
4-[(4-methoxyphenoxy)methyl]-1-[2-(pyrrolidin-1-yl)ethyl]-1H-1,2,3-triazole;
4-[(4-methoxyphenoxy)methyl]-1-[2-(morpholin-4-yl)ethyl]-1H,1,2,3-triazole;
4-[(4-methoxyphenoxy)methyl]-1-[2-(piperidin-1-yl)ethyl]-1H-1,2,3-triazole;
1-[2-(piperidin-1-yl)ethyl]-4-[(3-trifluoromethylphenoxy)methyl]-1H-1,2,3-triazole;
1-[2-(piperidin-1-yl)ethyl]-4-[(4-trifluoromethylphenoxy)methyl]-1H-1,2,3-triazole;
1-[2-(piperidin-1-yl)ethyl]-4-[(2-trifluoromethylphenoxy)methyl]-1H-1,2,3-triazole;
(guanidyl)ethyl]-4-[(4-methoxyphenoxy)methyl]-1H-1,2,3-triazole;
1-[2-(guanidyl)ethyl]-4-[(3-methoxyphenoxy)methyl]-1H-1,2,3-triazole;
1-[2-(guanidyl)ethyl]-4-[(3,4-methylendioxyphenoxy)methyl]-1H-1,2,3-triazole;
1-[2-(guanidyl)ethyl]-4-(phenoxymethyl)-1H-1,2,3-triazole;
1-[2-(guanidyl)ethyl]-4-[1-(4-methoxyphenoxy)-1-(methyl)-ethyl]-1H-1,2,3-triazole;
1-[2-(biguanidyl)ethyl]-4-[(4-methoxyphenoxy)methyl]-1H-1,2,3-triazole;
1-[2-(biguanidyl)ethyl]-4-[(3-methoxyphenoxy)methyl]-1H-1,2,3-triazole;
1-[2-(biguanidyl)ethyl]-4-[(3,4-methylendioxyphenoxy)methyl]-1H-1,2,3-triazole;
1-[2-(biguanidyl)ethyl]-4-(phenoxymethyl)-1H-1,2,3-triazole;
1-[2-(biguanidyl)ethyl]-4-[(4-methoxyphenoxy)-1-(methyl)-ethyl]-1H-1,2,3-triazole;
1-[2-(N,N-dimethylamino)ethyl]-4-[(4-hydroxyphenoxy)methyl]-1H-1,2,3-triazole;
5-iodo-4-[(4-methoxyphenoxy)methyl]-1-[2-(N,N-dimethylamino)ethyl]-1H-1,2,3-triazole;
1-[2-(N,N-dimethyl)-N-oxideaminoethyl]-4-[(4-methoxyphenoxy)methyl]-1H-1,2,3-triazole;
1-[2-(guanidyl)ethyl]-4-[(4-methoxyphenyl)thiomethyl]-1H-1,2,3-triazole;
1-[2-(biguanidinyl)ethyl]-4-[(4-methoxyphenyl)thiomethyl]-1H-1,2,3-triazole.
1-[3-(biguanidinyl)propyl]-4-[(4-methoxyphenyl)thiomethyl]-1H-1,2,3-triazole;

Even more preferably, the compound of formula (II) is selected from a group consisting of 1-[2-(guanidyl)ethyl]-4-[(4-methoxyphenyl)thiomethyl]-1H-1,2,3-triazole and 1-[2-(biguanidinyl)ethyl]-4-[(4-methoxyphenyl)thiomethyl]-1H-1,2,3-triazole.

Still more preferably, the compound of formula (II) is 1-[2-(biguanidinyl)ethyl]-4-[(4-methoxyphenyl)thiomethyl]-1H-1,2,3-triazole.

As shown in the examples and as explained for de compounds of formula (I), the compounds of formula (II) are also suitable for the treatment and/or prevention of ocular diseases.

Thus, another aspect relates to a compound of formula (II) or a pharmaceutically acceptable salt, stereoisomer, tautomer, solvate or N-oxide thereof, or an isotopically labeled derivative thereof as defined above for use in medicine.

Another aspect relates to a compound of formula (II) or a pharmaceutically acceptable salt, stereoisomer, tautomer, solvate or N-oxide thereof, or an isotopically labeled derivative thereof as defined above for use in the prevention and/or treatment of an ocular disease.

This aspect can also be worded as the use of a compound of formula (II) or a pharmaceutically acceptable salt, stereoisomer, tautomer, solvate or N-oxide thereof, or an isotopically labeled derivative thereof as defined above in the manufacture of a medicament for the prevention and/or treatment of an ocular disease.

This aspect can also be worded as a method of prevention and/or treatment of an ocular disease comprising the administration of a compound of formula (II) or a pharmaceutically acceptable salt, stereoisomer, tautomer, solvate or N-oxide thereof, or an isotopically labeled derivative thereof as defined above.

Preferably, the ocular disease is a retinal disease. More preferably, the retinal disease is selected from the group consisting of retinitis pigmentosa (such as autosomal dominant retinitis pigmentosa, autosomal recessive retinitis pigmentosa, X-linked retinitis pigmentosa, sporadic retinitis pigmentosa, retinitis pigmentosa associated with other syndromes), diabetic retinopathy, cone dystrophies, rod and cone degeneration, Leber congenital amaurosis, retinitis Punctata albescens, choroideremia, choroid and retina gyrate atrophy, generalized choroidal dystrophy, juvenile retinoschisis, Wagner vitreoretinal degeneration, autosomal dominant vitreoretinochoroidopathy, Stargardt disease, Best vitelliform macular dystrophy, Usher syndrome, Bardet-Biedl syndrome, Sorsby pseudoinflammatory macular dystrophy, age-related macular degeneration, autosomal dominant congenital stationary night blindness, achromatopsia and dominant drusen. More preferably, the ocular disease is selected from the group consisting of retinitis pigmentosa (such as autosomal dominant retinitis pigmentosa, autosomal recessive retinitis pigmentosa, X-linked retinitis pigmentosa, sporadic retinitis pigmentosa, retinitis pigmentosa associated with other syndromes), Stargardt disease, Leber congenital amaurosis, age-related macular degeneration and diabetic retinopathy. Still more preferably, the ocular disease is retinitis pigmentosa (such as autosomal dominant retinitis pigmentosa, autosomal recessive retinitis pigmentosa, X-linked retinitis pigmentosa, sporadic retinitis pigmentosa, retinitis pigmentosa associated with other syndromes).

In another preferred embodiment, the retinal disease is selected from retinitis pigmentosa, autosomal dominant congenital stationary night blindness and achromatopsia.

A further aspect relates to a pharmaceutical composition comprising a compound of formula (II) or a pharmaceutically acceptable salt, stereoisomer, tautomer, solvate or N-oxide thereof, or an isotopically labeled derivative thereof as defined above and a pharmaceutically acceptable excipient.

Pharmaceutically acceptable excipients, routes of administration and dosage forms are as previously defined. Preferably, the pharmaceutically acceptable excipient is selected from the group consisting of hyaluronic acid, carboxymethylcellulose, carbomer and mixtures thereof.

The compounds of formula (I) and formula (II) can be synthesized according to the procedures describe in WO 2017/203083 A1, in particular when Z is S, SO or SO₂.

When Z is O, the following general synthetic pathway can be used: a) react a w-chloroalkyl amine hydrochloride with sodium azide in water at 80° C. to form a solution of the corresponding o-azidoalkyl amine hydrochloride; b) mix the aqueous solution of the w-azidoalkyl amine intermediate with a terminal aryloxyalkyl alkyne in the presence of catalytic amounts of copper salts (e.g. copper(II) sulfate, copper(I) acetate, copper(I) iodide), optionally in the presence of a base (e.g. potassium carbonate, sodium acetate, triethylamine, N,N-diisopropylethylamine), optionally in the presence of a copper reducing agent (e.g. sodium ascorbate), optionally in the presence of added organic solvents (e.g. methanol, acetonitrile, tetrahydrofuran, tert-butanol); c) stir the reaction mixture at room temperature until completion (typically, overnight) and proceed with the work-up. These pathways are embodied in Example 1, Example 9 and Example 33.

When W is a guanidine derivative, the following general synthetic route may be used: a) prepare a 1-(ω-aminoalkyl)-1H-1,2,3-triazole as outlined above; b) react the primary amine with stoichiometric amounts of di-tert-butoxycarbonyl thiourea S═C(NHBoc)₂, molecular iodine and triethylamine in aprotic solvents (e.g. dichloromethane) at 0° C. until completion (typically, 2h); when W is an N-alkylated guanidine, use Boc-protected N-alkylthioureas S═C(NHBoc)[N(C₁-C₄-alkyl)Boc] or S═C[N(C₁-C₄-alkyl)Boc]₂ c) deprotect the resulting intermediate 1-[ω-(di-tert-butoxycarbonylguanidino)alkyl]-1H-1,2,3-triazole with a suitable acid (e.g. anhydrous HCl in 1,4-dioxane, trifluoroacetic acid). This synthetic route is detailed in Example 22.

When W is a biguanidine derivative, the following general synthetic route may be used: a) prepare a 1-(ω-aminoalkyl)-1H-1,2,3-triazole as outlined above; b) react the primary amine with stoichiometric amounts of cyanoguanidine and chlorotrimethylsilane in aprotic solvents (e.g. acetonitrile) at 150° C. using an ACE pressure tube until completion (typically, 15 min); when W is an N-alkylated biguanidine of the type —N(C₁-C₄ alkyl)C(═NH)NHC(═NH)NH₂, replace the primary amine by a 1-[w-(N-alkyl-amino)alkyl]-1H-1,2,3-triazole as embodied in Example 10; c) cool down the mixture, add isopropanol and recrystallize the hydrochloride salt. This synthetic route is detailed in Example 27.

### EXAMPLES

The following examples represent specific embodiments of the present invention.

### A) Synthesis of Compounds of Formula (I) Wherein Z is —O—

### Example 1: (Compound 1)

### 1-[2-(N,N-dimethylamino)ethyl]-4-[(4-methoxyphenoxy)methyl]-1H-1,2,3-triazole

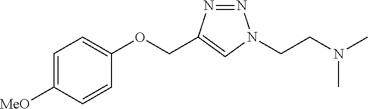 [SMILES: COc1ccc(OCc2cn(CCN(C)C)nn2)cc1]

A solution of 2-chloroethyl-N,N-dimethylammonium chloride (12 mmol, 1.72 g) and sodium azide (13 mmol, 0.84 g) in water (10 mL) was heated at 80° C. overnight. To the resulting solution of 2-azidoethyl-N,N-dimethylamine hydrochloride was added successively 4-methoxyphenyl propargyl ether (10 mmol, 1.63 g), dissolved in MeOH (40 mL), water (8 mL), CuOAc (0.5 mmol, 60 mg), NaOAc (30 mmol, 2.46 g) and sodium ascorbate (5 mmol, 0.99 g) and the mixture was stirred at 30° C. overnight. Organic solvents were evaporated and the remaining aqueous solution was stirred with 20% ammonia (25 mL) for 30 min and was extracted with EtOAc (3×50 mL). The organic phase was acidified with 2M HCl and the aqueous layer was washed with EtOAc (50 mL). The aqueous phase was basified with 20% Na₂CO₃ and extracted with EtOAc (3×50 mL). The combined organic extract was dried (Na₂SO₄) and evaporated at reduced pressure. The crude product was purified by column chromatography (silica gel, CH₂Cl₂/MeOH 95:5). Yield 2.62 g (95%). White solid (Mp: 68° C.). ¹H NMR (400 MHz, CDCl₃) δ 7.76 (s, 1H), 7.05-6.89 (m, 2H), 6.89-6.69 (m, 2H), 5.16 (s, 2H), 4.45 (t, J= 6.3 Hz, 2H), 3.77 (s, 3H), 2.77 (t, J=6.3 Hz, 2H), 2.28 (s, 6H). ¹³C NMR (126 MHz, CDCl₃): δ 154.0, 152.3, 144.1, 123.2, 115.7, 114.5, 62.6, 58.6, 55.6, 48.1, 45.3. IR (cm⁻¹): 3142, 2954, 2789, 2764, 1739, 1508, 1466, 1454, 1285, 1230, 1213, 1108, 1059, 1028, 940, 817, 779, 734, 661, 520. ESI-MS: HRMS: m/z calc for (M+H) C₁₄H₂₀N₄O₂ 276.1586, found 276.1591.

### Example 2: (Compound 2)

### 1-[2-(N,N-dimethylamino)ethyl]-4-[(3,4-methylendioxyphenoxy)methyl]-1H-1,2,3-triazole

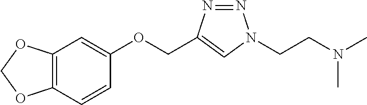 [SMILES: CN(C)CCn1cc(COc2ccc3c(c2)OCO3)nn1]

The procedure of Example 1 was followed starting from 3,4-methylenedioxyphenyl propargyl ether (10 mmol, 1.76 g) and 2-chloroethyl-N,N-dimethylammonium chloride (12 mmol, 1.72 g). Yield 2.34 g (80%). White solid (Mp: 44° C.). ¹H NMR (400 MHz, CDCl₃) δ 7.78 (s, 1H), 6.73 (d, J=8.5 Hz, 1H), 6.59 (d, J=2.5 Hz, 1H), 6.45 (dd, J=8.5 Hz, 2.5 Hz, 1H), 5.94 (s, 2H), 5.15 (s, 2H), 4.50 (t, J=6.3 Hz, 2H), 2.83 (t, J=6.3 Hz, 2H), 2.33 (s, 6H). ¹³C NMR (75 MHz, CDCl₃) δ 154.4, 148.9, 144.7, 142.7, 124.0, 108.6, 106.8, 101.8, 99.2, 63.7, 59.3, 48.8, 46.0. IR (cm⁻¹): 3139, 3101, 2953, 2884, 2824, 2765, 1738, 1638, 1610, 1500,1458, 1358,1281,1257,1189,1035, 926,836,800, 670,606, 541, 432. ESI-MS: HRMS: m/z calc for (M+H) C₁₄H₁₈N₄O₃ 290.1379, found 290.1384.

### Example 3: (Compound 3)

### 1-[2-(N,N-dimethylamino)ethyl]-4-[(3-methoxyphenoxy)methyl]-1H-1,2,3-triazole

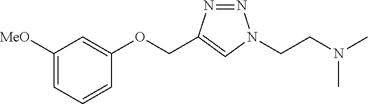 [SMILES: COc1cccc(OCc2cn(CCN(C)C)nn2)c1]

The procedure of Example 1 was followed starting from 3-methoxyphenyl propargyl ether (10.0 mmol, 1.78 g) and 2-chloroethyl-N,N-dimethylammonium chloride (12 mmol, 1.62 g). Yield 2.24 g (81%). Oil. ¹H NMR (400 MHz, CDCl₃) δ 7.79 (s, 1H), 7.21 (t, J=8.2 Hz, 2H), 6.65-6.53 (m, 3H), 5.21 (s, 2H), 4.47 (t, J=6.3 Hz, 2H), 3.80 (s, 3H), 2.79 (t, J=6.3 Hz, 2H), 2.30 (s, 6H). ¹³C NMR (101 MHz, CDCl₃) δ 161.1, 159.7, 144.2, 130.2, 123.6, 107.1, 107.0, 101.5, 62.3, 59.0, 55.5, 48.4, 45.6.

### Example 4: (Compound 4)

### 4-[(3,5-dimethoxyphenoxy)methyl]-1-[2-(N,N-dimethylamino)ethyl]-1H-1,2,3-triazole

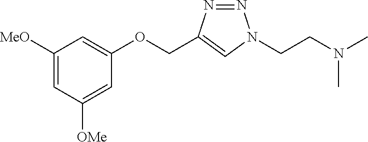 [SMILES: COc1cc(OC)cc(OCc2cn(CCN(C)C)nn2)c1]

The procedure of Example 1 was followed starting from 3,5-dimethoxyphenyl propargyl ether (10.0 mmol, 1.92 g) and 2-chloroethyl-N,N-dimethylammonium chloride (12 mmol, 1.72 g). Yield 1.80 g (59%). White solid (Mp: 43° C.). ¹H NMR (400 MHz, CDCl₃) δ 7.79 (s, 1H), 6.20 (d, J=2.1 Hz, 2H), 6.13 (t, J=2.2 Hz, 1H), 5.18 (s, 2H), 4.47 (t, J=6.3 Hz, 2H), 3.78 (s, 6H), 2.79 (t, J=6.3 Hz, 2H), 2.31 (s, 6H). ¹³C NMR (126 MHz, CDCl₃): δ 161.7, 160.3, 143.9, 123.5, 93.8, 93.7, 62.3, 58.9, 55.5, 48.4, 45.5. IR (cm⁻¹): 2946, 2767, 1589, 1449, 1394, 1361, 1205, 1147, 1051, 957, 815, 677, 538. ESI-MS: HRMS: m/z calc for (M+H) C₁₅H₂₂N₄O₃ 306.1692, found 306.1698.

### Example 5: (Compound 5)

### 1-[2-(N,N-dimethylamino)ethyl]-4-(phenoxymethyl)-1H-1,2,3-triazole

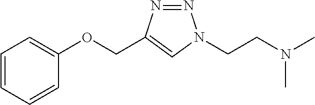 [SMILES: CN(C)CCn1cc(COc2ccccc2)nn1]

The procedure of Example 1 was followed starting from phenyl propargyl ether (10 mmol, 1.32 g) and 2-chloroethyl-N,N-dimethylammonium chloride (12 mmol, 1.72 g). Yield 1.88 g (76%). White solid (Mp 34° C.). ¹H NMR (400 MHz, CDCl₃) δ 7.78 (s, 1H), 7.35-7.25 (m, 2H), 7.05-6.89 (m, 2H), 5.22 (s, 2H), 4.45 (t, J=6.3 Hz, 2H), 2.78 (t, J=6.3 Hz, 2H), 2.29 (s, 6H).

### Example 6: (Compound 6)

### 1-[2-(N,N-dimethylamino)ethyl]-4-[(3-trifluoromethylphenoxy)methyl]-1H-1,2,3-triazole

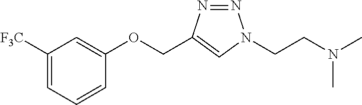 [SMILES: CN(C)CCn1cc(COc2cccc(C(F)(F)F)c2)nn1]

The procedure of Example 1 was followed starting from 3-trifluoromethylphenyl propargyl ether (5 mmol, 1.00 g) and 2-chloroethyl-N,N-dimethylammonium chloride (6 mmol, 0.86 g). Yield 1.41 g (90%). Pale yellow oil. ¹H NMR (400 MHz, CDCl₃) δ 7.80 (s, 1H), 7.36 (t, J=8.0 Hz, 1H), 7.24-7.07 (m, 3H), 5.20 (s, 2H), 4.42 (t, J=6.2 Hz, 2H), 2.73 (t, J=6.2 Hz, 2H), 2.24 (s, 6H).

### Example 7: (Compound 7)

### 1-[2-(N,N-dimethylamino)ethyl]-4-[(3,4,5-trimethoxyphenoxy)methyl]-1H-1,2,3-triazole

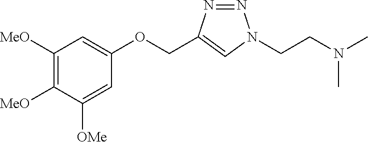 [SMILES: COc1cc(OCc2cn(CCN(C)C)nn2)cc(OC)c1OC]

The procedure of Example 1 was followed starting from 3,4,5-trimethoxyphenyl propargyl ether (5 mmol, 1.11 g) and 2-chloroethyl-N,N-dimethylammonium chloride (6 mmol, 0.86 g). Yield 1.44 g (86%). Pale yellow oil. ¹H NMR (400 MHz, CDCl₃) δ 7.72 (s, 1H), 6.18 (s, 2H), 5.07 (s, 2H), 4.34 (t, J=6.2, 2H), 3.72 (s, 6H), 3.67 (s, 3H), 2.66 (t, J=6.2, 2H), 2.17 (s, 6H). ¹³C NMR (126 MHz, CDCl₃): δ 155.0, 153.8, 144.0, 132.7, 123.5, 92.8, 62.6, 61.1, 58.8, 56.2, 48.4, 45.5. IR (cm⁻¹): 2941, 2824, 2772, 1737, 1592, 1504, 1458, 1420, 1226, 1192, 1124, 1046, 1029, 809, 730. ESI-MS: HRMS: m/z calc for (M+H) C₁₆H₂₄N₄O₄ 336.1798, found 336.1801.

### Example 8: (Compound 8)

### 4-[(3,4-dimethoxyphenoxy)methyl]-1-[2-(N,N-dimethylamino)ethyl]-1H-1,2,3-triazole

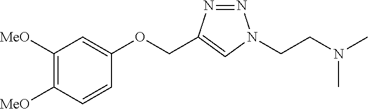 [SMILES: COc1ccc(OCc2cn(CCN(C)C)nn2)cc1OC]

The procedure of Example 1 was followed starting from 3,4-dimethoxyphenyl propargyl ether (10.0 mmol, 1.92 g) and 2-chloroethyl-N,N-dimethylammonium chloride (12 mmol, 1.72 g). Yield 1.89 g (62%). White solid (Mp: 39° C.). ¹H NMR (400 MHz, CDCl₃) δ 7.76 (s, 1H), 6.77 (d, J=8.6 Hz, 1H), 6.65 (s, 1H), 6.57 (d, J=8.5 Hz, 1H), 5.18 (s, 2H), 4.44 (t, J=6.3 Hz, 2H), 3.82 (s, 3H), 3.86 (s, 3H), 2.81 (t, J=6.3 Hz, 2H), 2.30 (s, 6H).

### Example 9: (Compound 9)

### 1-[2-(N,N-dimethylamino)ethyl]-4-[1-(4-methoxyphenoxy)-1-(methyl)-ethyl]-1H-1,2,3-triazole

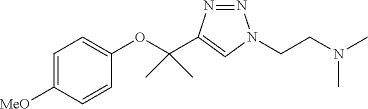 [SMILES: COc1ccc(OC(C)(C)c2cn(CCN(C)C)nn2)cc1]

To a solution of 2-methyl-3-butyn-2-ol (57.2 mmol, 5.59 mL) and DBU (74.6 mmol, 11.13 mL) in dry acetonitrile (50 mL) cooled at 0° C. under nitrogen atmosphere was added dropwise trifluoroacetic anhydride (57.2 mmol, 7.99 mL) and the reaction mixture was stirred at 0° C. for 30 minutes. This solution was added via canula to a stirred solution of 4-methoxyphenol (49.7 mmol, 6.17 g), DBU (64.6 mmol, 9.64 mL) and CuCl₂ (0.05 mmol, 6.7 mg) in dry acetonitrile (50 mL) cooled to −5° C. When the addition was completed, stirring was continued at room temperature for 16 hours. The organic solvent was evaporated in vacuo, the residue was redissolved in EtOAc and was successively washed with water (25 mL), 1 M HCl (25 mL) and brine (25 mL). Finally, the organic layer was dried (Na₂SO₄) and evaporated at reduced pressure to afford a crude product, which was used without further purification in subsequent reactions. 4-Methoxyphenyl 1,1-dimethyl-2-propynyl ether. Yield 9.00 g (95%). Black-brown oil. ¹H NMR (400 MHz, CDCl₃) δ 7.15 (d, J=9.0 Hz, 1H), 6.83 (d, J=9.0 Hz, 1H), 3.77 (s, 3H), 2.57 (s, 1H), 1.62 (s, 6H). ¹³C NMR (126 MHz, CDCl₃): δ 155.9, 149.0, 123.8, 114.0, 86.5, 73.9, 55.6, 29.6. IR (cm⁻¹): 3284, 2988, 2936, 2835, 1724, 1606, 1588, 1503, 1464, 1441, 1230, 1211, 1135, 1034, 949, 890, 843, 756, 733, 640, 532. The alkyne was reacted with 2-azidoethyl-N,N-dimethylammonium chloride following the procedure of Example 1. Yield 2.71 g (89%). White solid (Mp: 37° C.). ¹H NMR (400 MHz, CDCl₃) δ 7.47 (s, 1H), 6.59-6.51 (m, 4H), 4.30 (t, J=6.4 Hz, 2H), 3.60 (s, 3H), 2.62 (t, J=6.4 Hz, 2H), 1.92 (s, 6H), 1.60 (s, 6H).

### Example 10: (Compound 10)

### 4-[(4-methoxyphenoxy)methyl]-1-[2-(N-methylamino)ethyl]-1H-1,2,3-triazole

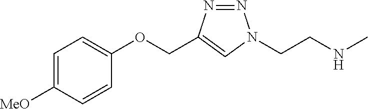 [SMILES: CNCCn1cc(COc2ccc(OC)cc2)nn1]

The procedure of Example 1 was followed starting from 4-methoxyphenyl propargyl ether (5.0 mmol, 815 mg) and 2-(N-methylamino)ethyl-1-ammonium chloride (7.0 mmol, 928 mg). Yield 917 mg (70%). Pale yellow solid (Mp: 34° C.). ¹H NMR (400 MHz, CD₃OD) δ 8.07 (s, 1H), 6.96 (d, J=9.1 Hz, 2H), 6.92-6.83 (m, 2H), 5.13 (s, 2H), 4.55 (t, J=6.2 Hz, 2H), 3.76 (s, 3H), 3.08 (t, J=6.2 Hz, 2H), 2.41 (s, 3H). ¹³C NMR (126 MHz, CDCl₃): δ 153.9, 148.4, 144.1, 142.2, 123.5, 108.1, 106.3, 101.4, 98.7, 63.2, 58.9, 48.4, 45.6. IR (cm⁻¹): 2942, 2835, 2799, 1738, 1505, 1462, 1364, 1217, 1108, 1033, 1009, 824, 733, 521. ESI-MS: HRMS: m/z calc for (M+H) C₁₃H₁₈N₄₄O₂ 262.1430, found 262.1432.

### Example 11: (Compound 11)

### 1-(2-aminoethyl)-4-[(4-methoxyphenoxy)methyl]-1H-1,2,3-triazole

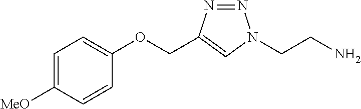 [SMILES: COc1ccc(OCc2cn(CCN)nn2)cc1]

The procedure of Example 1 was followed starting from 4-methoxyphenyl propargyl ether (20.0 mmol, 2.64 g), 2-chloroethyl-1-ammonium chloride (30.0 mmol, 3.47 g). Yield 3.02 g (61%). Pale brown solid (Mp: 66.5-68.0° C.). ¹H NMR (400 MHz, CD₃OD) δ 8.05 (s, 1H), 6.95 (d, J=9.1 Hz, 2H), 6.86 (d, J=9.2 Hz, 2H), 5.11 (s, 2H), 4.46 (t, J=6.1 Hz, 2H), 3.75 (s, 3H), 3.12 (t, J=6.2 Hz, 2H).

### Example 12: (Compound 12)

### 1-(2-aminoethyl)-4-[(3-methoxyphenoxy)methyl]-1H-1,2,3-triazole

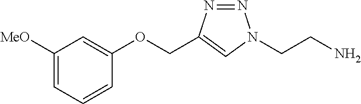 [SMILES: COc1cccc(OCc2cn(CCN)nn2)c1]

The procedure of Example 1 was followed starting from 3-methoxyphenyl propargyl ether (10.0 mmol, 1.62 g), 2-chloroethyl-1-ammonium chloride (15.0 mmol, 1.78 g). Yield 1.54 g (62%). Pale brown solid. ¹H NMR (400 MHz, CD₃OD) δ 8.06 (s, 1H), 7.18 (t, J=8.1 Hz, 2H), 6.68-6.51 (m, 3H), 5.15 (s, 2H), 4.46 (t, J=6.2 Hz, 2H), 3.76 (s, 3H), 3.12 (t, J=6.1 Hz, 2H).

### Example 13: (Compound 13)

### 1-(2-aminoethyl)-4-[(3,4-methylendioxyphenoxy)methyl]-1H-1,2,3-triazole

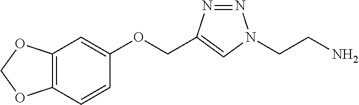 [SMILES: NCCn1cc(COc2ccc3c(c2)OCO3)nn1]

The procedure of Example 1 was followed starting from 3,4-methylenedioxyphenyl propargyl ether (10 mmol, 1.92 g) and 2-chloroethyl-1-ammonium chloride (15.0 mmol, 1.78 g). Yield 1.75 g (67%). Orange solid (Mp: 56° C.). ¹H NMR (400 MHz, CD₃OD) δ 8.0 (s, 1H), 6.71 (d, J=8.5 Hz, 1H), 6.59 (d, J=2.6 Hz, 1H), 6.46 (dd, J=8.5 Hz, 2.5 Hz, 1H), 5.89 (s, 2H), 5.09 (s, 2H), 4.84 (s, 1H), 4.46 (t, J=6.1 Hz, 2H), 3.13 (t, J=6.1 Hz, 3H). ¹³C NMR (126 MHz, CDCl₃): δ 153.8, 148.4, 144.1, 142.2, 123.5, 108.0, 106.2, 101.3, 98.5, 63.0, 53.5, 42.0. IR (cm⁻¹): 3365, 2943, 2922, 1619, 1483, 1389, 1359, 1266, 1237, 1181, 1133, 1099, 1057, 1035, 998, 937, 838, 810, 780, 737, 610, 511, 437. ESI-MS: HRMS: m/z calc for (M+H) C₁₃H₁₈N₄O₂ 262.1430, found 262.1432.

### Example 14: (Compound 14)

### 1-(2-aminoethyl)-4-(phenoxymethyl)-1H-1,2,3-triazole

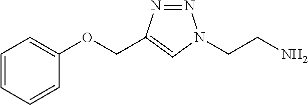 [SMILES: NCCn1cc(COc2ccccc2)nn1]

The procedure of Example 1 was followed starting from phenyl propargyl ether (10 mmol, 1.32 g) and 2-chloroethyl-1-ammonium chloride (15.0 mmol, 1.78 g). Yield 1.13 g (52%). Pale yellow solid (Mp: 34° C.). ¹H NMR (400 MHz, CD₃OD) δ 8.09 (s, 1H), 7.37-7.02 (m, 5H), 5.19 (s, 2H), 4.48 (t, J=6.1, 2H), 3.14 (s, 2H). ¹³C NMR (126 MHz, CDCl₃): δ 158.3, 144.3, 129.7, 123.6, 121.4, 114.9, 62.1, 53.4, 41.9. IR (cm⁻¹): 3338, 2873, 1738, 1636, 1599, 1586, 1561, 1484, 1427, 1384, 1338, 1226, 1173, 1079, 1052, 1007, 843, 818, 748, 690, 511. ESI-MS: HRMS: m/z calc for (M+H) C₁₁H₁₄N₄O 218.1168, found 218.1168.

### Example 15: (Compound 15)

### 1-(2-aminoethyl)-4-[1-(4-methoxyphenoxy)-1-(methyl)-ethyl]-1H-1,2,3-triazole

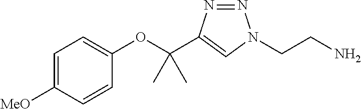 [SMILES: COc1ccc(OC(C)(C)c2cn(CCN)nn2)cc1]

The procedure of Example 1 was followed starting from 4-methoxyphenyl 1,1-dimethyl-2-propynyl ether (10 mmol, 1.90 g) prepared in Example 9, and 2-chloroethyl-1-ammonium chloride (15.0 mmol, 1.78 g). Yield 2.00 g (72%). Brown oil. ¹H NMR (400 MHz, CD₃OD) δ 7.87 (s, 1H), 6.76-6.67 (m, 2H), 6.67-6.59 (m, 2H), 4.43 (t, J=6.2 Hz, 2H), 3.72 (s, 3H), 3.09 (t, J=6.2 Hz, 2H), 1.73 (s, 6H). ¹³C NMR (126 MHz, CDCl₃): δ 156.1, 153.1, 148.8, 124.6, 122.4, 114.3, 77.0, 56.0, 53.6, 42.4, 28.1. IR (cm⁻¹): 3366, 2981, 2935,1502,1463,1441,1381,1365,1290,1214,1134,1032,940,924,875, 844, 799, 732. ESI-MS: HRMS: m/z calc for (M+H) C₁₄H₂₀N₄O₂ 276.1586, found 276.1589.

### Example 16: (Compound 16)

### 4-[(4-methoxyphenoxy)methyl]-1-[2-(pyrrolidin-1-yl)ethyl]-1H-1,2,3-triazole

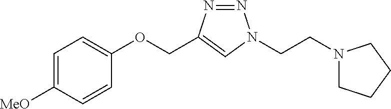 [SMILES: COc1ccc(OCc2cn(CCN3CCCC3)nn2)cc1]

The procedure of Example 1 was followed starting from 4-methoxyphenyl propargyl ether (5 mmol, 811 mg) and 1-(2-chloroethyl)pyrrolidine hydrochloride (7.5 mmol, 1.38 g). Yield 1.47 g (97%). White solid (Mp: 57° C.). ¹H NMR (400 MHz, CDCl₃) δ 7.75 (s, 1H), 6.93 (d, J=9. Hz, 2H), 6.84 (d, J=9.1 Hz, 2H), 5.17 (s, 2H), 4.47 (t, J=6.2 Hz, 2H), 3.77 (s, 3H), 3.67 (t, J=4.6 Hz, 4H), 2.83 (t, J=6.2 Hz, 2H), 2.49 (t, J=4.5 Hz, 4H). ¹³C NMR (126 MHz, CDCl₃): δ 154.4, 152.5, 144.5, 123.4, 116.0, 114.8, 67.0, 63.0, 58.0, 55.9, 53.7, 47.6. IR (cm⁻¹): 3134, 2958, 2780, 2102, 1737, 1507, 1459, 1436, 1350, 1284, 1213, 1108, 1028, 933, 819, 791, 734, 666, 518. ESI-MS: HRMS: m/z calc for (M+H) C₁₆H₂₂N₄O₂ 302.1743, found 302.1741.

### Example 17: (Compound 17)

### 4-[(4-methoxyphenoxy)methyl]-1-[2-(morpholin-4-yl)ethyl]-1H,1,2,3-triazole

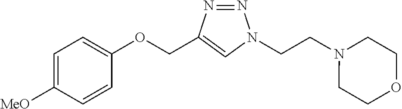 [SMILES: COc1ccc(OCc2cn(CCN3CCOCC3)nn2)cc1]

The procedure of Example 1 was followed starting from 4-methoxyphenyl propargyl ether (5 mmol, 811 mg) and 1-(2-chloroethyl)morpholine hydrochloride (7.5 mmol, 1.38 g). Yield 1.24 g (78%). White solid (Mp: 78° C.). ¹H NMR (400 MHz, CDCl₃) δ 7.75 (s, 1H), 7.00-6.89 (m, 2H), 6.84 (dd, J=8.6, 1.5, 2H), 5.17 (d, J=1.6, 2H), 4.47 (td, J=6.2, 1.5, 3H), 3.77 (d, J=1.3, 3H), 3.72-3.63 (m, 4H), 2.83 (td, J=6.2, 1.7, 2H), 2.49 (t, J=4.5, 4H). ¹³C NMR (126 MHz, CDCl₃): δ 154.4, 152.5, 144.5, 123.4, 116.1, 114.8, 67.0, 63.0, 58.0, 55.9, 53.7, 47.6. IR (cm⁻¹): 3074, 2862, 1738, 1508, 1456, 1377, 1218, 1148, 1113, 1033, 916, 869, 820, 798, 716, 553, 518. ESI-MS: HRMS: m/z calc for (M+H) C₁₆H₂₂N₄O₃ 318.1692, found 318.1697.

### Example 18: (Compound 18)

### 4-[(4-methoxyphenoxy)methyl]-1-[2-(piperidin-1-yl)ethyl]-1H-1,2,3-triazole

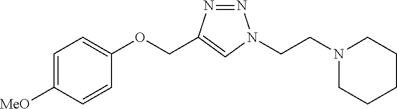 [SMILES: COc1ccc(OCc2cn(CCN3CCCCC3)nn2)cc1]

The procedure of Example 1 was followed starting from 4-methoxyphenyl propargyl ether (5 mmol, 811 mg) and 1-(2-chloroethyl)piperidine hydrochloride (7.5 mmol, 1.35 g). Yield 1.43 g (90%). Yellowish oil. ¹H NMR (400 MHz, CDCl₃) δ 7.78 (s, 1H), 7.01-6.90 (m, 2H), 6.88-6.80 (m, 2H), 5.17 (s, 2H), 4.45 (t, J=6.4 Hz, 2H), 3.77 (s, 3H), 2.76 (t, J=6.4 Hz, 2H), 2.42 (t, J=5.4 Hz, 4H), 1.57 (dq, J=16.7, 5.6 Hz, 4H), 1.45 (q, J=5.9 Hz, 2H). ¹³C NMR (126 MHz, CDCl₃): δ 154.1, 152.3, 144.1, 123.3, 115.8, 114.6, 62.7, 58.1, 55.6, 54.4, 47.8, 25.9, 24.1. IR (cm⁻¹): 3087, 2932, 2777, 2098, 1738, 1509, 1467, 1440, 1305, 1229, 1146, 1107, 1051, 1032, 853, 818, 787, 709, 548, 512. ESI-MS: HRMS: m/z calc for (M+H) C₁₇H₂₄N₄O₂ 316.1899, found 316.1901.

### Example 19: (Compound 19)

### 1-[2-(piperidin-1-yl)ethyl]-4-[(3-trifluoromethylphenoxy)methyl]-1H-1,2,3-triazole

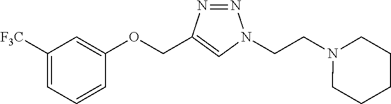 [SMILES: FC(F)(F)c1cccc(OCc2cn(CCN3CCCCC3)nn2)c1]

The procedure of Example 1 was followed starting from 3-trifluoromethylphenyl propargyl ether (5 mmol, 1.00 g) and 1-(2-chloroethyl)piperidine hydrochloride (7.5 mmol, 1.35 g). Yield 1.58 g (89%). Oil. ¹H NMR (400 MHz, CDCl₃) δ 7.83 (s, 1H), 7.39 (t, J=8.0 Hz, 2H), 7.25-7.15 (m, 2H), 5.25 (s, 2H), 4.46 (t, J=6.3 Hz, 2H), 2.75 (t, J=6.3 Hz, 2H), 2.41 (t, J=5.2 Hz, 4H), 1.54 (p, J=5.5 Hz, 4H), 1.48-1.37 (m, 2H).

### Example 20: (Compound 20)

### 1-[2-(piperidin-1-yl)ethyl]-4-[(4-trifluoromethylphenoxy)methyl]-1H-1,2,3-triazole

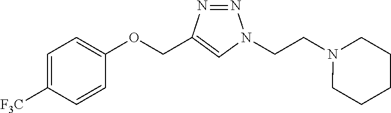 [SMILES: FC(F)(F)c1ccc(OCc2cn(CCN3CCCCC3)nn2)cc1]

The procedure of Example 1 was followed starting from 4-trifluoromethylphenyl propargyl ether (5 mmol, 1.00 g) and 1-(2-chloroethyl)piperidine hydrochloride (7.5 mmol, 1.35 g). Yield 1.62 g (92%). Oil. ¹H NMR (400 MHz, CDCl₃) δ 7.83 (s, 1H), 7.56 (d, J=8.5 Hz, 2H), 7.09 (d, J=8.5 Hz, 2H), 5.28 (s, 2H), 4.49 (t, J=6.3 Hz, 2H), 2.78 (t, J=6.3 Hz, 2H), 2.43 (t, J=5.4 Hz, 4H), 1.56 (p, J=5.5 Hz, 4H), 1.52-1.41 (m, 2H).

### Example 21: (Compound 21)

### 1-[2-(piperidin-1-yl)ethyl]-4-[(2-trifluoromethylphenoxy)methyl]-1H-1,2,3-triazole

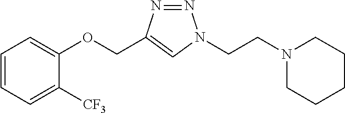 [SMILES: FC(F)(F)c1ccccc1OCc1cn(CCN2CCCCC2)nn1]

The procedure of Example 1 was followed starting from 2-trifluoromethylphenyl propargyl ether (5 mmol, 1.00 g) and 1-(2-chloroethyl)piperidine hydrochloride (7.5 mmol, 1.35 g). Yield 1.58 g (90%). Oil. ¹H NMR (400 MHz, CDCl₃) δ 7.84 (s, 1H), 7.59-7.54 (m, 1H), 7.52-7.44 (m, 1H), 7.17 (d, J=8.3 Hz, 1H), 7.01 (t, J=7.6 Hz, 1H), 5.33 (s, 2H), 4.44 (t, J=6.2 Hz, 2H), 2.73 (t, J=6.2 Hz, 2H), 2.40 (t, J=5.4 Hz, 4H), 1.53 (p, J=5.5 Hz, 4H), 1.47-1.35 (m, 2H).

### Example 22: (Compound 22)

### (guanidyl)ethyl]-4-[(4-methoxyphenoxy)methyl]-1H-1,2,3-triazole

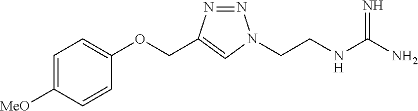 [SMILES: COc1ccc(OCc2cn(CCNC(=N)N)nn2)cc1]

1-[(2-Aminoethyl)-4-[(4-methoxyphenoxy)methyl]-1H-1,2,3-triazole (1 mmol, 248 mg) prepared in Example 11, di-tert-butoxycarbonyl thiourea (1.2 mmol, 332 mg), I₂ (1.2 mmol, 305 mg) and triethylamine (1.2 mmol, 121 mg) were dissolved in CH₂Cl₂ (10 mL) and the mixture was stirred for two hours at 0° C. Volatiles were evaporated under reduced pressure, a saturated solution of NH₄Cl (10 mL) was added and the aqueous solution was extracted with CH₂Cl₂ (3×10 mL). The organic layer was dried (Na₂SO₄) and concentrated under reduced pressure. The crude product was purified by column chromatography (CH₂Cl₂/MeOH 98:02; CH₂Cl₂/MeOH 95:05) to afford the intermediate 1-[2-(N,N′-di-tert-butoxycarbonylguanidino)ethyl]-4-[(4-methoxyphenoxy)methyl]-1H-1,2,3-triazole. Yield 251 mg (51%). Pale yellow solid. ¹H NMR (300 MHz, CDCl₃) δ 11.43 (s, 1H), 8.57 (t, J=5.8 Hz, 1H), 7.71 (s, 1H), 6.92 (d, J=9.2 Hz, 2H), 6.82 (d, J=9.2 Hz, 2H), 5.16 (s, 2H), 4.59 (d, J=6.5, 2H), 3.93 (q, J=5.9, 2H), 3.76 (s, 3H), 1.51 (s, 9H), 1.47 (s, 9H). ¹³C NMR (126 MHz, CDCl₃) δ 162.8, 156.2, 154.0, 152.8, 152.2, 144.5, 123.2, 115.7, 114.5, 83.5, 79.6, 62.6, 55.5, 49.1, 40.5, 28.1, 27.9. IR (cm⁻¹): 3326, 2979, 2932, 1721, 1637, 1614, 1567, 1506, 1413, 1366, 1324, 1226, 1133, 1097, 1038, 911, 823, 731. ESI-MS: HRMS: m/z calc for (M+H) C₂₃H₃₄N₆O₆ 490.2542, found 490.2540. To a solution of the intermediate dicarbamate compound (0.5 mmol, 245 mg) in CH₂Cl₂ (5 mL) was added anhydrous HCl (4.0 M in dioxane, 1.1 mmol, 0.27 mL) and the reaction mixture was stirred at 0-5° C. for 4 h. Upon completion, the volatiles were evaporated at reduced pressure and the residue was washed with anhydrous Et₂O to provide the hydrochloride salt of the product. Yield 147 mg (90%). Pale yellow solid. ¹H NMR (400 MHz, CD₃OD) δ 7.99 (s, 1H), 6.84 (d, J=9.1 Hz, 2H), 6.75 (d, J=9.1 Hz, 2H), 5.02 (s, 2H), 4.52 (t, J=6.5, 2H), 3.68 (t, J=6.1, 2H), 3.65 (s, 3H). ¹³C NMR (75 MHz, CD₃OD) δ 158.8, 155.8, 153.7, 145.6, 125.8, 117.0, 115.7, 63.0, 56.1, 50.1, 42.1. IR (cm⁻¹): 3304, 2955, 1670, 1619, 1619, 1506, 1464, 1440, 1199, 1181, 1132, 1034, 827, 799, 721. ESI-MS: HRMS: m/z calc for (M+H) C₁₃H₁₈N₆O₂ 290.1491, found 290.1496.

### Example 23: (Compound 23)

### 1-[2-(guanidyl)ethyl]-4-[(3-methoxyphenoxy)methyl]-1H-1,2,3-triazole

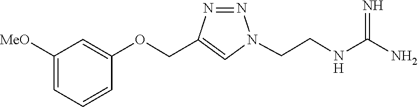 [SMILES: COc1cccc(OCc2cn(CCNC(=N)N)nn2)c1]

The procedure of Example 22 was followed starting from 1-(2-aminoethyl)-4-[(3-methoxyphenoxy)methyl]-1H-1,2,3-triazole (1 mmol, 248 mg) prepared according to Example 12 to afford the intermediate 1-[2-(N,N′-di-tert-butoxycarbonylguanidino)ethyl]-4-[(3-methoxyphenoxy)methyl]-1H-1,2,3-triazole. Yield 305 mg (62%). Pale yellow solid. ¹H NMR (400 MHz, CDCl₃) δ 11.42 (s, 1H), 8.57 (s, 1H), 7.73 (s, 1H), 7.32-7.15 (m, 1H), 6.65-6.50 (m, 3H), 5.18 (s, 2H), 4.58 (t, J=5.9, 2H), 3.94 (t, J=5.9, 2H), 3.77 (s, 3H), 1.49 (s, 9H), 1.46 (s, 9H). ¹³C NMR (126 MHz, CDCl₃) δ 162.9, 160.6, 159.3, 156.1, 152.7, 144.0, 129.8, 123.3, 106.8, 106.6, 101.1, 83.4, 79.5, 61.8, 55.1, 49.1, 40.4, 28.1, 27.8. IR (cm⁻¹): 3326, 3146, 2978, 2935, 1721, 1638, 1612, 1492, 1413, 1366, 1325, 1133, 1097, 1043, 1018, 731. ESI-MS: HRMS: m/z calc for (M+H) C₂₃H₃₄N₆O₆490.2540, found 490.2542. Deprotection of the dicarbamate (0.5 mmol, 245 mg) provided the product. Yield 49 mg (97%). Pale yellow solid. ¹H NMR (400 MHz, CD₃OD) δ 8.01 (s, 1H), 7.08 (t, J=8.1, 1H), 6.58-6.39 (m, 3H), 5.06 (s, 2H), 4.53 (dt, J=6.5, 2H), 3.69 (t, J=6.0, 2H), 3.66 (s, 3H). ¹³C NMR (75 MHz, CD₃OD) δ 162.3, 160.8, 158.8, 145.3, 131.0, 125.9, 107.9, 107.8, 102.3, 62.3, 55.7, 50.1, 42.1. IR (cm⁻¹): 3331, 2942, 2376, 1671, 1593, 1492, 1454,1436, 1284,1263,1197,1135,1043, 974,834,799, 764,721. ESI-MS: HRMS: m/z calc for (M+H) C₁₃H₁₈N₆O₂ 290.2542, found 290.254.

### Example 24: (Compound 24)

### 1-[2-(guanidyl)ethyl]-4-[(3,4-methylendioxyphenoxy)methyl]-1H-1,2,3-triazole

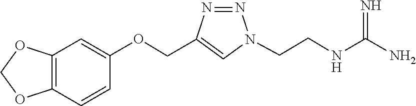 [SMILES: N=C(N)NCCn1cc(COc2ccc3c(c2)OCO3)nn1]

The procedure of Example 22 was followed starting from 1-(2-aminoethyl)-4-[3,4-(methylenedioxy)phenoxymethyl]-1H-1,2,3-triazole (1 mmol, 262 mg) which was prepared according to Example 13. Yield 288 mg (57%). Pale yellow solid. ¹H NMR (400 MHz, CDCl₃) δ 11.43 (s, 1H), 8.60 (s, 1H), 7.71 (s, 1H), 6.70 (d, J=8.5, 1H), 6.56 (d, J=2.3, 1H), 6.42 (ddd, J=8.4, 2.6, 0.9, 2H), 5.92 (s, 2H), 5.13 (s, 2H), 4.60 (t, J=5.9, 2H), 3.95 (q, J=6.1 Hz, 2H), 1.51 (s, 9H), 1.48 (s, 9H). ¹³C NMR (126 MHz, CDCl₃) δ 162.8, 156.1, 153.4, 152.7, 148.0, 144.0, 141.8, 123.3, 107.7, 105.85, 101.0, 98.3, 83.4, 79.5, 62.7, 49.0, 40.4, 28.0, 27.8. IR (cm⁻¹): 3327, 3138, 2978, 2933, 1720, 1613, 1563, 1486, 1413,1366,1324,1228, 1177,1131,1097,1034,1016, 916,809,775, 730, 611. ESI-MS: HRMS: m/z calc for (M+H) C₂₃H₃₂N₆O₇ 504.2332, found 504.2331. Deprotection of the dicarbamate (0.5 mmol, 252 mg) provided the product. Yield 162 mg (95%). Pale yellow solid. ¹H NMR (400 MHz, CD₃OD) δ 7.99 (s, 1H), 6.60 (d, J=8.5 Hz, 1H), 6.47 (d, J=2.5 Hz, 1H), 6.34 (dd, J=8.4, 2.6 Hz, 1H), 5.78 (s, 2H), 4.99 (s, 2H), 4.53 (t, J= 5.8, 2H), 3.69 (t, J=5.8, 2H). ¹³C NMR (75 MHz, CD₃OD) δ 158.7, 154.9, 149.6, 145.2, 143.4, 125.8, 108.8, 107.2, 102.4, 99.3, 63.2, 63.2, 50.1, 42.0. IR (cm⁻¹): 3336, 2896, 2377, 1670, 1612, 1502, 1486, 1363, 1178, 1131, 1035, 925, 834, 799, 721. ESI-MS: HRMS: m/z calc for (M+H) C₁₃H₁₆N₆O₃ 304.1355, found 304.1360.

### Example 25: (Compound 25)

### 1-[2-(guanidyl)ethyl]-4-(phenoxymethyl)-1H-1,2,3-triazole

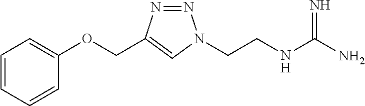 [SMILES: N=C(N)NCCn1cc(COc2ccccc2)nn1]

The procedure of Example 22 was followed starting from 1-(2-aminoethyl)-4-(phenoxymethyl)-1H-1,2,3-triazole (0.80 mmol, 176 mg) which was prepared according to Example 14. Yield 266 mg (71%). White solid. ¹H NMR (400 MHz, CDCl₃) δ 11.45 (s, 1H), 8.64 (s, 1H), 7.74 (s, 1H), 7.43-7.22 (m, 2H), 7.11-6.92 (m, 3H), 5.25 (s, 2H), 4.62 (t, J=5.9 Hz, 2H), 3.98 (q, J=5.9 Hz, 2H), 1.54 (s, 9H), 1.51 (s, 9H). Deprotection of the dicarbamate (0.40 mmol, 109 mg) provided the product. Yield 175 mg (95%). White solid. ¹H NMR (400 MHz, CD₃OD) δ 8.11 (s, 1H), 7.38-6.91 (m, 5H), 5.20 (s, 2H), 4.72 (t, J=6.5, 2H), 3.88 (t, J=6.1, 2H).

### Example 26: (Compound 26)

### 1-[2-(guanidyl)ethyl]-4-[1-(4-methoxyphenoxy)-1-(methyl)-ethyl]-1H-1,2,3-triazole

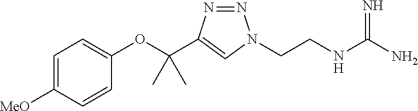 [SMILES: COc1ccc(OC(C)(C)c2cn(CCNC(=N)N)nn2)cc1]

The procedure of Example 22 was followed starting from 1-(2-aminoethyl)-4-[1-(4-methoxyphenoxy)-1-(methyl)-ethyl]-1H-1,2,3-triazole (1 mmol, 276 mg) which was prepared according to Example 15. Yield 108 mg (34%). Oil. ¹H NMR (400 MHz, CD₃OD) δ 7.01 (s, 1H), 7.22 (d, J=8.7 Hz, 2H), 6.88 (d, J=8.6 Hz, 2H), 4.57 (t, J=6.3 Hz, 2H), 3.74 (s, 3H), 3.62 (t, J=6.3 Hz, 2H), 1.70 (s, 6H).

### Example 27: (Compound 27)

### 1-[2-(biguanidyl)ethyl]-4-[(4-methoxyphenoxy)methyl]-1H-1,2,3-triazole

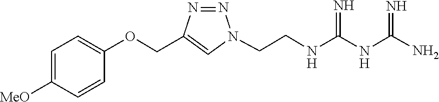 [SMILES: COc1ccc(OCc2cn(CCNC(=N)NC(=N)N)nn2)cc1]

A mixture of 1-(2-aminoethyl)-4-[(4-methoxyphenoxy)methyl]-1H-1,2,3-triazole (1 mmol, 248 mg), cyanoguanidine (1 mmol, 84 mg), acetonitrile (1.35 mL) and chlorotrimethylsilane (1.1 mmol, 0.14 mL) placed in an ACE pressure tube was stirred for 15 minutes in a silicone oil bath heated to 150° C. Then, the ACE tube was cooled to 50° C. for 10 minutes, isopropanol (3 mmol, 0.23 mL) was added, and the mixture was heated again to 125° C. for one minute by using another silicone oil bath. Finally, the reaction mixture was cooled down to room temperature, the resulting hydrochloride solid product was filtered off and further purified by column chromatography, when necessary. Yield: 218 mg, (57%). Oil. ¹H NMR (400 MHz, CD₃OD) δ 8.07 (s, 1H), 7.35 (d, J=8.3 Hz, 2H), 6.90 (d, J=8.2 Hz, 2H), 4.80 (d, J=5.4 Hz, 2H), 4.19 (s, 2H), 3.80 (s, 3H), 3.54 (d, J=5.3 Hz, 2H). ¹³C NMR (101 MHz, CD₃OD) δ 164.6, 161.2, 159.0, 145.9, 135.3, 126.5, 125.7, 115.8, 55.9, 49.3, 40.0, 30.6.

### Example 28: (Compound 28)

### 1-[2-(biguanidyl)ethyl]-4-[(3-methoxyphenyl)thiomethyl]-1H-1,2,3-triazole

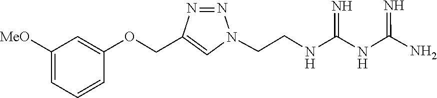 [SMILES: COc1cccc(OCc2cn(CCNC(=N)NC(=N)N)nn2)c1]

The procedure of Example 27 was followed starting from 1-(2-aminoethyl)-4-[(3-methoxyphenoxy)methyl]-1H-1,2,3-triazole (1 mmol, 248 mg). Yield: 236 mg, (71%). Oil. ¹H NMR (400 MHz, CD₃OD) δ 8.53 (s, 1H), 7.35-7.18 (m, 1H), 6.67-6.55 (m, 2H), 5.19 (s, 2H), 4.80 (t, J=5.4 Hz, 2H), 3.94 (d, J=5.3 Hz, 2H), 3.79 (s, 3H).

### Example 29: (Compound 29)

### 1-[2-(biguanidyl)ethyl]-4-[(3-methoxyphenyl)thiomethyl]-1H-1,2,3-triazole

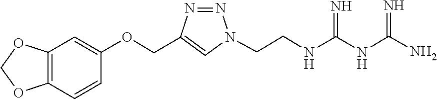 [SMILES: N=C(N)NC(=N)NCCn1cc(COc2ccc3c(c2)OCO3)nn1]

The procedure of Example 27 was followed starting from 1-(2-aminoethyl)-4-[3,4-(methylenedioxy)phenoxymethyl]-1H-1,2,3-triazole (1 mmol, 262 mg). Yield: 152 mg, (44%). Oil. ¹H NMR (400 MHz, CD₃OD) δ 7.81 (s, 1H), 6.62 (d, J=8.4 Hz, 1H), 6.48 (d, J=2.5 Hz, 1H), 6.35 (dd, J=8.4, 2.5 Hz, 1H), 5.79 (s, 2H), 4.94 (s, 2H), 4.55 (t, J=5.7, 2H), 3.65 (t, J=5.7, 2H).

### Example 30: (Compound 30)

### 1-[2-(biguanidyl)ethyl]-4-(phenoxymethyl)-1H-1,2,3-triazole

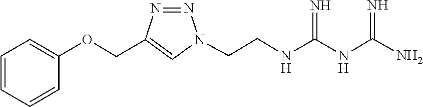 [SMILES: N=C(N)NC(=N)NCCn1cc(COc2ccccc2)nn1]

The procedure of Example 27 was followed starting from 1-(2-aminoethyl)-4-(phenoxymethyl)-1H-1,2,3-triazole (1.00 mmol, 218 mg). Yield: 148 mg, (49%). Oil. ¹H NMR (400 MHz, CD₃OD) δ 8.06 (s, 1H), 7.39-6.89 (m, 5H), 5.21 (s, 2H), 4.72 (t, J=6.4, 2H), 3.90 (t, J=6.4, 2H).

### Example 31: (Compound 31)

### 1-[2-(biguanidyl)ethyl]-4-[(3-methoxyphenyl)thiomethyl]-1H-1,2,3-triazole

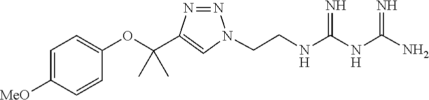 [SMILES: COc1ccc(OC(C)(C)c2cn(CCNC(=N)NC(=N)N)nn2)cc1]

The procedure of Example 27 was followed starting from 1-(2-aminoethyl)-4-[1-(4-methoxyphenoxy)-1-(methyl)-ethyl]-1H-1,2,3-triazole (1 mmol, 276 mg). Yield 100 mg (28%). Oil. ¹H NMR (400 MHz, CD₃OD) δ 7.94 (s, 1H), 7.24 (d, J=8.7 Hz, 2H), 6.82 (d, J=8.7 Hz, 2H), 4.55 (t, J=6.3 Hz, 2H), 3.79 (s, 3H), 3.66 (t, J=6.3 Hz, 2H), 1.76 (s, 6H).

### Example 32: (Compound 32)

### 1-[2-(N,N-dimethylamino)ethyl]-4-[(4-hydroxyphenoxy)methyl]-1H-1,2,3-triazole

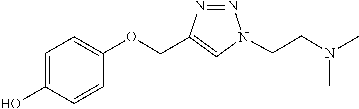 [SMILES: CN(C)CCn1cc(COc2ccc(O)cc2)nn1]

A solution of 1-[2-(N,N-dimethylamino)ethyl]-4-[(4-methoxyphenoxy)methyl]-1H-1,2,3-triazole (1 mmol, 278 mg), thiophenol (1 mmol, 0.103 mL) and K₂CO₃ (cat, 7 mg) in N-methyl pyrrolidone (1 mL) was heated under nitrogen to 200° C. for 2 hours. The solvent was evaporated in vacuo and the residue was purified by column chromatography (silica gel; CH₂Cl₂/MeOH (7M NH₃) 95:5). Yield 160 mg (61%). ¹H NMR (400 MHz, CDCl₃) δ 7.49 (s, 1H), 7.05-6.95 (m, 2H), 6.89-6.78 (m, 2H), 5.08 (s, 2H), 4.47 (t, J=6.4 Hz, 2H), 2.71 (t, J=6.4 Hz, 2H), 2.30 (s, 6H).

### Example 33: (Compound 33)

### 5-iodo-4-[(4-methoxyphenoxy)methyl]-1-[2-(N,N-dimethylamino)ethyl]-1H-1,2,3-triazole

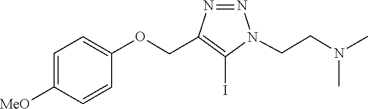 [SMILES: COc1ccc(OCc2nnn(CCN(C)C)c2I)cc1]

To a solution of 4-methoxyphenyl propargyl ether (3 mmol, 486 mg), CuI (3.4 mmol, 649 mg), NBS (3.6 mmol, 3.6 mg), DIPEA (3.4 mmol, 591 μL) in dry CH₃CN (8 mL) kept under nitrogen atmosphere, was added 2-azido-N,N-dimethylethyl-1-amine (3.3 mmol, 376 mg) and the reaction mixture was stirred at room temperature for 2 h. Then, solvent was evaporated under reduced pressure, the residue was suspended in aq sat NaCl (10 mL) and the aqueous suspension was extracted with CH₂Cl₂ (3×10 mL). The combined organic layers were washed with aq sat NaCl (10 mL), dried over MgSO₄ and concentrated under reduced pressure. The product was purified by column chromatography. (CH₂Cl₂/MeOH 20:1). Yield 0.85 g (70%) as brown solid. ¹H NMR (400 MHz, CDCl₃) δ 7.05-6.93 (m, 2H), 6.89-6.79 (m, 2H), 5.07 (s, 2H), 4.48 (t, J=7.2 Hz, 2H), 3.76 (s, 3H), 2.82 (t, J=7.2 Hz, 2H), 2.32 (s, 6H). ¹³C NMR (101 MHz, CDCl₃): δ 154.1, 152.2, 147.2, 116.1, 114.4, 81.3, 62.4, 58.2, 55.5, 48.5, 45.4, 45.2. IR (cm⁻¹): 2994, 2937, 2827, 2795, 2768, 1712, 1506, 1461, 1452, 1287, 1215, 1130, 1081, 1035, 1005, 926, 860, 821, 804, 707, 523. ESI-MS: HRMS: m/z calc for (M+H) C₁₄H₁₉IN₄O 2402.0553, found 402.0562.

### Example 34: (Compound 34)

### 1-[2-(N,N-dimethyl)-N-oxideaminoethyl]-4-[(4-methoxyphenoxy)methyl]-1H-1,2,3-triazole

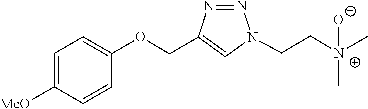 [SMILES: COc1ccc(OCc2cn(CC[N+](C)(C)[O-])nn2)cc1]

A solution of 1-[2-(N,N-dimethylamino)ethyl]-4-[(4-methoxyphenoxy)methyl]-1H-1,2,3-triazole (1 mmol, 276 mg) and 33% aqueous hydrogen peroxide (1 mL) in MeOH (4 mL) was stirred at room temperature overnight. The solvents were evaporated to below 25° C. using an efficient vacuum pump. Yield: 289 mg (98%). Colorless oil. ¹H NMR (400 MHz, CD₃OD) δ 8.18 (s, 1H), 6.94 (d, J=9.1 Hz, 2H), 6.86 (d, J=9.2 Hz, 2H), 5.13 (s, 2H), 5.03 (t, J=6.5 Hz, 2H), 3.96 (t, J=6.5 Hz, 2H), 3.75 (s, 3H), 3.23 (s, 6H).

### B) Synthesis of Compounds of Formula (I) Wherein Z is —S—

### Example 35: (Compound 35)

### 1-[2-(N,N-dimethylamino)ethyl]-4-[(4-methoxyphenyl)thiomethyl]-1H-1,2,3-triazole

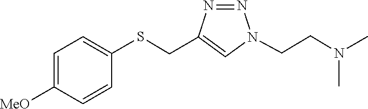 [SMILES: COc1ccc(SCc2cn(CCN(C)C)nn2)cc1]

The procedure of Example 1 was followed starting from 4-methoxyphenyl propargyl sulfide (30 mmol, 5.35 g) and 2-chloroethyl-N,N-dimethylammonium hydrochloride (36 mmol, 5.18 g). Yield 7.53 g (86%). White solid (Mp: 35-40° C.). ¹H NMR (400 MHz, CD₃OD) δ 7.67 (s, 1H), 7.29 (d, J=8.7 Hz, 2H), 6.85 (d, J=6.9 Hz, 2H), 4.44 (t, J=6.5 Hz, 2H), 4.09 (s, 2H), 3.77 (s, 3H), 2.75 (t, J=6.5 Hz, 2H), 2.27 (s, 6H). ¹³C RMN (101 MHz, CDCl₃): 159.2, 144.8, 133.7, 125.5, 122.6, 114.5, 58.6, 55.3, 48.0, 45.3, 30.9. ESI-MS: HRMS: m/z for(M+H) C₁₄H₂₁N₄OS 293.1436; found: 293,1440.

### Example 36: (Compound 36)

### 1-[3-(N,N-dimethylamino)propyl]-4-[(4-methoxyphenyl)thiomethyl]-1H-1,2,3-triazole

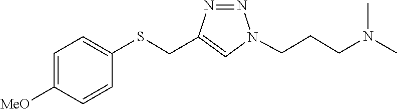 [SMILES: COc1ccc(SCc2cn(CCCN(C)C)nn2)cc1]

The procedure of Example 1 was followed starting from 4-methoxyphenyl propargyl sulfide (10 mmol, 1.91 g) and 3-chloropropyl-N,N-dimethylammonium hydrochloride (12 mmol, 1.89 g). Yield 2.31 g (70%). Oil. ¹H NMR (400 MHz, CD₃OD) δ 7.64 (s, 1H), 7.30 (d, J=8.8 Hz, 2H), 6.86 (d, J=8.7 Hz, 2H), 4.37 (t, J=6.9 Hz, 2H), 4.09 (s, 2H), 3.78 (s, 3H), 2.25 (t, J=5.4 Hz, 2H), 2.22 (s, 6H), 2.01 (q, J=8.1, 6.5 Hz, 2H). ¹³C NMR (101 MHz, CD₃OD) δ 164.9, 138.3, 134.5, 128.6, 127.8, 116.8, 57.1, 54.5, 54.4, 50.4, 49.0, 12.9. IR (cm⁻¹): 2942, 2818, 2767, 1591, 1492, 1459, 1283, 1242, 1174, 1027, 824, 637, 522.

### Example 37: (Compound 37)

### 1-[2-(N,N-diethylamino)ethyl]-4-[(4-methoxyphenyl)thiomethyl]-1H-1,2,3-triazole

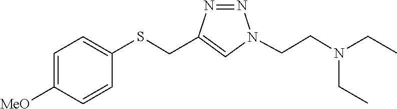 [SMILES: CCN(CC)CCn1cc(CSc2ccc(OC)cc2)nn1]

The procedure of Example 1 was followed starting from 4-methoxyphenyl propargyl sulfide (15 mmol, 2.67 g) and 2-chloroethyl-N,N-diethylammonium hydrochloride (18.0 mmol, 3.10 g). Yield 2.74 g (57%). Brown oil. ¹H NMR (400 MHz, CD₃OD) δ 7.66 (s, 1H), 7.29 (d, J=8.8 Hz, 2H), 6.84 (d, J=8.8 Hz, 2H), 4.37 (t, J=6.5 Hz, 2H), 4.09 (s, 2H), 3.74 (s, 3H), 2.83 (t, J=6.5 Hz, 2H), 2.50 (q, J=7.1 Hz, 4H), 0.95 (t, J=s, 6H). ¹³C NMR (101 MHz, CD₃OD): δ 161.7, 146.6, 135.8, 127.5, 125.8, 116.6, 56.7, 54.4, 50.3, 49.0, 32.1, 13.0. IR (cm⁻¹): 2967, 2810, 1738, 1591, 1492, 1460, 1283, 1241, 1174, 1027, 823, 637, 522.

### Example 38: (Compound 38)

### 1-[2-(piperidin-1-yl)ethyl]-4-[(3-(trifluoromethylphenyl)thiomethyl]-1H-1,2,3-triazole

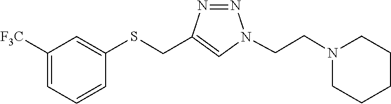 [SMILES: FC(F)(F)c1cccc(SCc2cn(CCN3CCCCC3)nn2)c1]

The procedure of Example 1 was followed starting from 3-trifluoromethylyphenyl propargyl sulfide (5 mmol, 1.08 g) and 1-(2-chloroethyl)piperidine hydrochloride (7.5 mmol, 1.35 g). Yield 1.67 g (90%). Yellowish oil. ¹H NMR (400 MHz, CD₃OD) δ 7.89 (s, 1H), 7.65-7.57 (m, 2H), 7.53-7.45 (m, 2H), 4.47 (td, J=6.6, 1.3 Hz, 2H), 4.33 (s, 2H), 2.75 (td, J=6.6, 1.8 Hz, 2H), 2.47-2.36 (m, 4H), 1.52 (q, J=5.3 Hz, 4H), 1.48-1.36 (m, 2H).

### Example 39: (Compound 39)

### 4-[(4-methoxyphenyl)thiomethyl]-1-[2-(piperidin-1-yl)ethyl]-1H-1,2,3-triazole

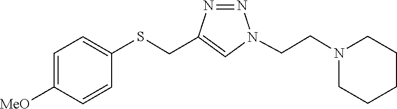 [SMILES: COc1ccc(SCc2cn(CCN3CCCCC3)nn2)cc1]

The procedure of Example 1 was followed starting from 4-methoxyphenyl propargyl sulfide (10 mmol, 1.78 g) and 1-(2-chloroethyl)piperidine hydrochloride (12 mmol, 2.16 g). Yield 2.59 g (78%). Oil. ¹H NMR (400 MHz, CDCl₃) δ 7.45 (s, 1H), 7.32 (d, J=8.7 Hz, 2H), 6.82 (d, J=8.7 Hz, 2H), 4.39 (t, J=6.4 Hz, 2H), 4.14 (s, 2H), 3.79 (s, 3H), 2.71 (t, J=6.4 Hz, 2H), 2.48-2.33 (m, 4H), 1.63-1.50 (m, 4H), 1.50-1.37 (m, 2H).

### Example 40: (Compound 40)

### 4-[(4-tert-butylphenyl)thiomethyl]-1-[2-(piperidin-1-yl)ethyl]-1H-1,2,3-triazole

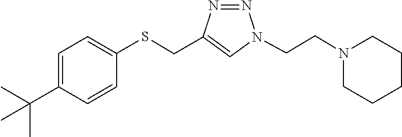 [SMILES: CC(C)(C)c1ccc(SCc2cn(CCN3CCCCC3)nn2)cc1]

The procedure of Example 1 was followed starting from 4-tert-butylphenyl propargyl sulfide (5 mmol, 1.02 g) and 1-(2-chloroethyl)piperidine hydrochloride (7 mmol, 1.26 g). Yield 1.13 g (63%). Oil. ¹H NMR (400 MHz, CD₃OD) δ 7.79 (s, 1H), 7.35 (d, J=8.6 Hz, 2H), 7.29 (d, J=8.4 Hz, 2H), 4.47 (t, J=6.5 Hz, 2H), 4.20 (s, 3H), 2.76 (t, J=6.5 Hz, 2H), 2.49-2.36 (m, 4H), 1.62-1.53 (m, 4H), 1.51-1.40 (m, 2H), 1.32 (s, 9H).

### Example 41: (Compound 41)

### 4-[(4-isopropylphenyl)thiomethyl]-1-[2-(4-morpholinyl)ethyl]-1H,1,2,3-triazole

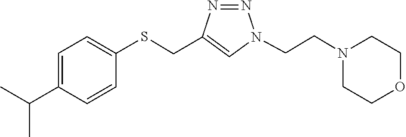 [SMILES: CC(C)c1ccc(SCc2cn(CCN3CCOCC3)nn2)cc1]

The procedure of Example 1 was followed starting from 4-isopropylphenyl propargyl sulfide (7 mmol, 1.33 g) and 4-(2-chloroethyl)morpholine hydrochloride (9 mmol, 1.67 g). Yield 2.16 g (89%). White solid (Mp: 66-68° C.). ¹H NMR (400 MHz, CDCl₃) δ 7.53 (s, 1H), 7.30 (d, J=8.3 Hz, 2H), 7.16 (d, J=8.2 Hz, 2H), 4.42 (t, J=6.2 Hz, 2H), 4.25 (s, 2H), 3.71-3.58 (m, 4H), 2.89 (hept, J=6.9 Hz, 1H), 2.78 (t, J=6.2 Hz, 2H), 2.50-2.38 (m, 4H), 1.25 (d, J=6.9 Hz, 6H).

### Example 42: (Compound 42)

### 1-[2-(4-morpholinyl)ethyl]-4-(phenylthiomethyl)-1H,1,2,3-triazole

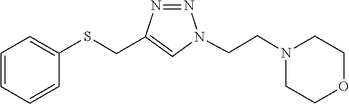 [SMILES: c1ccc(SCc2cn(CCN3CCOCC3)nn2)cc1]

The procedure of Example 1 was followed starting from phenyl propargyl ether (4.69 mmol, 697 mg) and 4-(2-chloroethyl)morpholine hydrochloride (5.7 mmol, 1.05 g). Yield 985 mg (69%). White solid (Mp: 57-59° C.). ¹H NMR (400 MHz, CDCl₃) δ 7.51 (s, 1H), 7.40-7.12 (m, 5H), 4.39 (t, J=6.1 Hz, 2H), 4.26 (s, 2H), 3.69-3.58 (m, 4H), 2.75 (t, J=6.1 Hz, 2H), 2.47-2.39 (m, 4H).

### Example 43: (Compound 43)

### 1-[2-(N,N-dimethylamino)ethyl]-4-[(4-hydroxyphenyl)thiomethyl]-1H-1,2,3-triazole

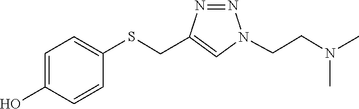 [SMILES: CN(C)CCn1cc(CSc2ccc(O)cc2)nn1]

The procedure of Example 32 was followed starting from 1-[2-(N,N-dimethylamino)ethyl]-4-[(4-methoxyphenyl)thiomethyl]-1H-1,2,3-triazole (1 mmol, 292 mg). Yield. (57%). White solid Mp: 102° C. ¹H NMR (400 MHz, CDCl₃) δ 7.49 (s, 1H), 7.16 (d, J=8.5 Hz, 2H), 6.67 (d, J=8.5 Hz, 2H), 4.47-4.37 (m, 2H), 4.05 (s, 2H), 2.76 (t, J=6.4 Hz, 2H), 2.28 (s, 6H). ¹³C NMR (101 MHz, CDCl₃) δ 157.1, 145.2, 134.5, 122.9, 122.9, 116.3, 58.4, 48.1, 45.2, 30.7.

### Example 44: (Compound 44)

### 1-[1-(R)-benzyl-2-(piperidin-1-yl)ethyl]-4-[(4-methoxyphenyl)thiomethyl]-1H-1,2,3-triazole

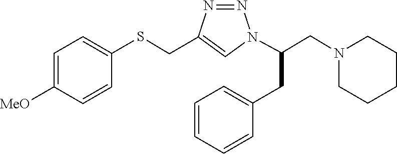 [SMILES: COc1ccc(SCc2cn([C@H](Cc3ccccc3)CN3CCCCC3)nn2)cc1]

To a solution of (R)-(+)-2-amino-3-phenyl-1-propanol (19.9 mmol, 3.00 g) in dry CH₂Cl₂ (20 mL) under nitrogen atmosphere, triethylamine (20.8 mmol, 2.88 mL) and di-tert-butyl dicarbonate (20.75 mmol, 4.53 g) were added at 0° C. Then, the reaction mixture was stirred at room temperature overnight and was successively washed with sat NH₄Cl (3×15 mL) and brine (2×15 mL). Yield: 4.44 g (89%). ¹H NMR (400 MHz, CDCl₃) δ 7.36-7.20 (m, 5H), 3.95-3.83 (m, 1H), 3.69 (dd, J=11.0, 3.8 Hz, 1H), 3.58 (dd, J=11.0, 5.3 Hz, 1H), 2.87 (d, J=7.2 Hz, 2H), 1.44 (s, 9H). Methanesulfonyl chloride (53.0 mmol, 7.35 mL) was added dropwise to a solution of (R)—N-tert-butoxycarbonyl-1-hydroxy-3-phenylpropyl-2-amine (17.67 mmol, 4.44 g) and triethylamine (53.0 mmol, 4.10 mL) in anhydrous THE (20 mL) cooled to 0° C. under nitrogen, and the mixture was stirred at the same temperature for one hour. The resulting suspension was basified with NaHCO₃ (sat. 20 mL) and the product was extracted with CH₂Cl₂ (3×15 mL). The crude mesylated product (17.67 mmol) was immediately dissolved in dry CH₃CN (7 mL) under nitrogen atmosphere and triethylamine (35.34 mmol, 4.90 mL) and piperidine (70.68 mmol, 10.86 mL) were added. The reaction mixture was stirred at room temperature for 48 hours. After evaporation of the solvent under vacuum, the reaction crude was dissolved in CH₂Cl₂ (20 mL) and was extracted with 10% citric acid (3×10 mL). The aqueous phase was basified with NaHCO₃ and the product was extracted with CH₂Cl₂ (3×15 mL). Finally, the product was purified by column chromatography (EtOAc/Hex 1:1). Yield: 3.35 g (59%). ¹H NMR (300 MHz, CDCl₃) δ 7.37-7.17 (m, 5H), 4.06-3.89 (m, 1H), 2.97 (dd, J=13.6, 5.4 Hz, 1H), 2.91-2.74 (m, 1H), 2.60-2.34 (m, 5H), 2.27 (dd, J=12.4, 6.2 Hz, 1H), 1.61 (d, J=5.2 Hz, 4H), 1.52-1.37 (m, 11H). To a solution of (R)-1-(2-tert-butoxycarbonylamino-3-phenylpropyl)-piperidine (7.85 mmol, 2.5 g) was dissolvent in a mixture of CH₂Cl₂ (2 mL) and trifluoroacetic acid (4 mL) and was stirred at room temperature for 1.5 hours. Then, the solvents were evaporated in vacuo, 1 M NaOH (5 mL) was added and the product was extracted with CH₂Cl₂ (3×4 mL). To a solution of (R)-1-(2-amino-3-phenylpropyl)-piperidine (7.85 mmol) in MeOH (5 mL) under nitrogen atmosphere, a solution of CuSO₄—H₂O (0.078 mmol, 19 mg) in H₂O (0.5 mL) and KHCO₃ (15.7 mmol, 1.57 g) were added. Finally, a freshly prepared solution of trifluoromethanesulfonyl azide (17 mmol) in CH₂Cl₂ (25 mL) was added and the reaction mixture was stirred at room temperature for 3 hours. Organic solvents were evaporated at reduced pressure, the aqueous solution was basified with NaHCO₃ (sat. 10 mL) and extracted with CH₂Cl₂ (3×15 mL). The combined organic phases were dried (NaSO₄), evaporated, and the (R)-1-(2-azido-3-phenylpropyl)-piperidine product was purified by column chromatography (CH₂Cl₂/MeOH 95:5). Yield: 480 mg (25%, overall). ¹H NMR (400 MHz, CDCl₃) δ 7.39-7.25 (m, 4H), 3.79 (tt, J=8.2, 4.9 Hz, 1H), 2.86 (dd, J=13.9, 5.1 Hz, 1H), 2.74 (dd, J=13.9, 8.1 Hz, 1H), 2.48 (dddt, J=16.8, 11.3, 8.4, 4.5 Hz, 6H), 1.70-1.59 (m, 4H), 1.47 (p, J=6.1 Hz, 2H). The procedure of Example 1 was followed to synthesize the 1,2,3-triazole ring, starting from 4-methoxyphenyl propargyl sulfide (2.2 mmol, 392 mg) and (R)-1-(2-azido-3-phenylpropyl)-piperidine (2 mmol, 489 mg). Yield 584 mg (66%). Oil. ¹H NMR (400 MHz, CD₃OD) δ 7.55 (s, 1H), 7.26-7.17 (m, 5H), 6.99 (dd, J=7.4, 2.1 Hz, 2H), 6.83 (d, J=8.8 Hz, 2H), 4.90-4.84 (m, 1H), 4.03 (s, 2H), 3.78 (s, 3H), 3.27-3.05 (m, 2H), 2.98-2.74 (m, 2H), 2.52-2.37 (m, 2H), 2.34-2.22 (m, 2H), 1.54-1.47 (m, 4H), 1.47-1.36 (m, 2H).

### Example 45: (Compound 45)

### 1-[1-(R)-benzyl-2-(piperidin-1-yl)ethyl]-4-[(3-trifluoromethylphenyl)thiomethyl]-1H-1,2,3-triazole

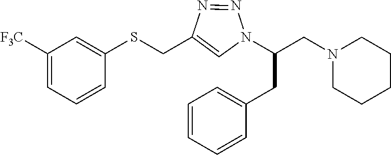 [SMILES: FC(F)(F)c1cccc(SCc2cn([C@H](Cc3ccccc3)CN3CCCCC3)nn2)c1]

The procedure of Example 1 was followed starting from 3-trifluoromethylphenyl propargyl sulfide (2.2 mmol, 476 mg) and (R)-1-(2-azido-3-phenylpropyl)-piperidine (2 mmol, 489 mg). Yield 749 mg (74%). Oil. ¹H NMR (400 MHz, CD₃OD) δ 7.68 (s, 1H), 7.31 (t, J=8.0 Hz, 2H), 7.25-7.20 (m, 2H), 7.17-7.14 (m, 3H), 6.95 (dd, J=6.8, 2.9 Hz, 2H), 4.90-4.80 (m, 1H), 3.30-3.18 (m, 2H), 2.93-2.70 (m, 2H), 2.41 (dd, J=6.8, 5.4 Hz, 2H), 2.29 (dd, J=11.1, 5.4 Hz, 2H), 1.44 (t, J=5.6 Hz, 4H), 1.39-1.33 (m, 2H).

### Example 46: (Compound 46)

### 1-[1-(R)-benzyl-2-(piperidin-1-yl)ethyl]-4-[(4-tert-butylphenyl)thiomethyl]-1H-1,2,3-triazole

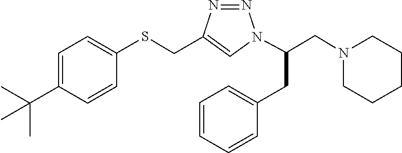 [SMILES: CC(C)(C)c1ccc(SCc2cn([C@H](Cc3ccccc3)CN3CCCCC3)nn2)cc1]

The procedure of Example 1 was followed starting from 4-tert-butylphenyl propargyl sulfide (2.2 mmol, 449 mg) and (R)-1-(2-azido-3-phenylpropyl)-piperidine (2 mmol, 489 mg). Yield 592 mg (60%). Oil. ¹H NMR (400 MHz, CD₃OD) δ 7.60 (s, 1H), 7.31 (d, J=8.5 Hz, 2H), 7.22 (d, J=8.5 Hz, 2H), 7.17-7.13 (m, 3H), 6.95 (dd, J=6.7, 2.9 Hz, 2H), 4.92-4.82 (m, 1H), 3.25-3.06 (m, 2H), 2.95-2.75 (m, 2H), 2.41 (dd, J=6.8, 5.8 Hz, 2H), 2.24 (dd, J=11.0, 5.4 Hz, 2H), 1.46 (t, J=5.5 Hz, 4H), 1.41-1.34 (m, 2H), 1.30 (s, 9H).

### Example 47: (Compound 47)

### 1-[3-(N,N-diethylamino)-2-methyl-propyl]-4-[(4-methoxyphenyl)thiomethyl]-1H-1,2,3-triazole

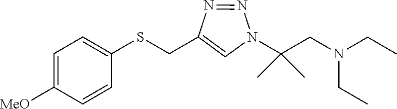 [SMILES: CCN(CC)CC(C)(C)n1cc(CSc2ccc(OC)cc2)nn1]

To a solution of sodium azide (45 mmol, 2.92 g) and sodium iodide (0.75 mmol, 112 mg) in DMSO, methyl α-bromoisobutyrate (15 mmol, 1.94 mL) was added. The reaction mixture was stirred at 50° C. for 2 hours. The product was extracted with diethyl ether (3×10 mL). Yield: 1.78 g (83%). Brown oil. ¹H NMR (400 MHz, CDCl₃) δ 3.82 (s, 3H), 1.51 (s, 6H). The procedure of Example 1 was followed starting from 4-methoxyphenyl propargyl sulfide (11 mmol, 1.96 g) and methyl 2-azido-2-methylpropanoate (13.2 mmol, 1.89 g). Yield 3.39 g (96%). Brown oil. ¹H NMR (400 MHz, CDCl₃) δ 7.36 (s, 1H), 7.31 (d, J=8.7 Hz, 2H), 6.83 (d, J=8.7 Hz, 2H), 4.14 (s, 2H), 3.79 (s, 3H), 3.72 (s, 3H), 1.90 (s, 6H). To a solution of 1-(1-methoxycarbonyl-1-methylethyl)-4-[(4-methoxyphenyl)thiomethyl]-1H-1,2,3-triazole (5.29 mmol, 1.70 g) in H₂O/THF (17:17.25 mL), LiOH·H₂O (10.58 mmol, 443 mg) was added and the reaction mixture was stirred at room temperature overnight. THE was evaporated in vacuum and after acidifying the aqueous phase, the product was extracted with EtOAc (3×10 mL). Yield: 1.21 g (74%). Brown oil. ¹H NMR (400 MHz, CD₃OD) δ 7.70 (s, 1H), 7.32-7.25 (m, 2H), 6.94-6.80 (m, 2H), 4.08 (s, 2H), 3.79 (s, 3H), 1.88 (s, 6H). To a solution of 1-(1-carboxy-1 -methylethyl)-4-[(4-methoxyphenyl)thiomethyl]-1H-1,2,3-triazole (3.04 mmol, 935 mg) in dry CH₂Cl₂ (9 mL) under N₂, N,N-dimethylformamide (0.304 mmol, 23 μL) and oxalyl chloride (3.65 mmol, 309 μL) were dropwise added at 0° C. The reaction mixture was stirred at room temperature for one hour. Then, N,N-diethylamine (6.08 mmol, 629 μL) was added dropwise and the new reaction mixture was stirred at room temperature overnight. The product was purified by column chromatography (CH₂Cl₂/MeOH 98:2). Yield: 825 mg (75%). ¹H NMR (400 MHz, CDCl₃) δ 7.32 (s, 1H), 7.29 (d, J=2.7 Hz, 2H), 6.82 (d, J=8.8 Hz, 2H), 4.16 (s, 2H), 3.80 (s, 3H), 3.37-3.25 (m, 2H), 2.76-2.66 (m, 2H), 1.86 (s, 6H), 1.17-1.03 (m, 3H), 0.79-0.69 (m, 3H).

To a suspension of 1-[1-(N,N-diethylaminocarbonyl)-1-methylethyl]-4-[(4-methoxyphenyl)thiomethyl]-1H-1,2,3-triazole (0.55 mmol, 200 mg) in dry Et₂O (5 mL) under N₂, a solution of LiAlH₄ (1.10 mmol, 41.87 mg) in dry Et₂O (5 mL). The reaction mixture was stirred at room temperature overnight. Cautiously, H₂O (1 mL) was added and the solution was filtered, dried over MgSO₄ and the solvent was evaporated. The product was purified by column chromatography (CH₂Cl₂/MeOH 98:2). Yield: 162 mg (84%). ¹H NMR (400 MHz, CDCl₃) δ 7.42 (s, 1H), 7.34 (d, J=8.8 Hz, 2H), 6.84 (d, J=8.8 Hz, 2H), 4.16 (s, 2H), 3.80 (s, 3H), 2.68 (s, 2H), 2.22 (q, J=7.1 Hz, 4H), 1.60 (s, 6H), 0.82 (t, J=7.1 Hz, 6H).

### Example 48: (Compound 48)

### 1-[2-(guanidyl)ethyl]-4-[(4-methoxyphenyl)thiomethyl]-1H-1,2,3-triazole

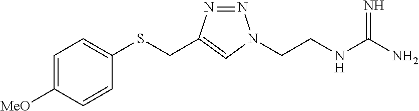 [SMILES: COc1ccc(SCc2cn(CCNC(=N)N)nn2)cc1]

The procedure of Example 22 was followed starting from 1-(2-aminoethyl)-4-[(4-methoxyphenyl)thiomethyl]-1H-1,2,3-triazole (0.76 mmol, 200 mg). Intermediate 1-[2-(N,N′-di-tert-butoxycarbonylguanidino)ethyl]-4-[(4-methoxyphenyl)thiomethyl]-1H-1,2,3-triazole. Yield 276 mg (72%). ¹H NMR (400 MHz, CDCl₃) δ 11.45 (s, 1H), 8.52 (t, J=5.9 Hz, 1H), 7.36 (s, 1H), 7.31 (d, J=8.8 Hz, 2H), 6.82 (d, J=8.8 Hz, 2H), 4.56-4.48 (m, 2H), 4.12 (s, 2H), 3.87 (q, J=5.9 Hz, 2H), 3.79 (s, 3H), 1.52 (s, 9H), 1.48 (s, 9H). Deprotection of the intermediate (0.25 mmol, 125 mg) with trifluoroacetic acid (1.25 mL) in CH₂Cl₂ (3.75 mL) provided the title product. Yield 73 mg (96%). Oil. ¹H NMR (400 MHz, CD₃OD) δ 7.82 (s, 1H), 7.32 (d, J=8.8 Hz, 2H), 6.88 (d, J=8.8 Hz, 2H), 4.58 (t, J=5.8 Hz, 2H), 4.11 (s, 2H), 3.79 (s, 3H), 3.75 (t, J=5.8 Hz, 2H).

### Example 49: (Compound 49)

### 1-[2-(biguanidinyl)ethyl]-4-[(4-methoxyphenyl)thiomethyl]-1H-1,2,3-triazole

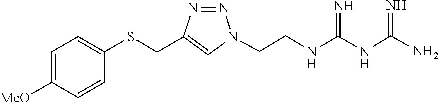 [SMILES: COc1ccc(SCc2cn(CCNC(=N)NC(=N)N)nn2)cc1]

The procedure of Example 27 was followed starting from 1-(2-aminoethyl)-4-[4-(methoxy)phenylthiomethyl]-1H-1,2,3-triazole (0.38 mmol, 100 mg), cyanoguanidine (0.38 mmol, 31.8 mg), acetonitrile (0.5 mL), chlorotrimethylsilane (0.42 mmol, 0.05 mL) and isopropanol (1.14 mmol, 0.09 mL). Yield: 83 mg, (57%). Oil. ¹H NMR (400 MHz, CD₃OD) δ 8.07 (s, 1H), 7.35 (d, J=8.3 Hz, 2H), 6.90 (d, J=8.2 Hz, 2H), 4.80 (d, J=5.4 Hz, 2H), 4.19 (s, 2H), 3.80 (s, 3H), 3.54 (d, J=5.3 Hz, 2H). ¹³C NMR (101 MHz, CD₃OD) δ 164.6, 161.2, 159.0, 145.9, 135.3, 126.5, 125.7, 115.8, 55.9, 49.3, 40.0, 30.6.

### Example 50: (Compound 50)

### 1-[3-(biguanidinyl)propyl]-4-[(4-methoxyphenyl)thiomethyl]-1H-1,2,3-triazole

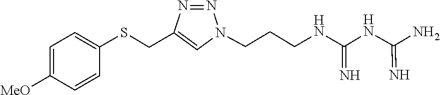 [SMILES: COc1ccc(SCc2cn(CCCNC(=N)NC(=N)N)nn2)cc1]

The procedure of Example 27 was followed starting from 1-(3-aminopropyl)-4-[4-(methoxy)phenylthiomethyl]-1H-1,2,3-triazole (0.36 mmol, 100 mg), cyanoguanidine (0.36 mmol, 30.2 mg), acetonitrile (0.5 mL), chlorotrimethylsilane (0.40 mmol, 0.05 mL) and isopropanol (1.08 mmol, 0.08 mL). Yield: 64.6 mg, (45%). Oil. ¹H NMR (400 MHz, CD₃OD) δ 8.33 (s, 1H), 7.43-7.29 (m, 2H), 6.97-6.84 (m, 2H), 4.24 (s, 2H), 3.81 (s, 3H), 3.04 (dd, J=9.2, 6.2 Hz, 2H), 2.35 (p, J=7.1 Hz, 2H). ¹³C NMR (101 MHz, CD₃OD) δ 164.6, 161.6, 159.0, 144.4, 136.0, 127.9, 124.5, 116.1, 55.9, 50.6, 37.7, 29.6, 28.5.

### Example 51: (Compound 51)

### 1-[2-(N,N-dimethylamino)ethyl]-5-iodo-4-[(4-methoxyphenyl)thiomethyl]-1H-1,2,3-triazole

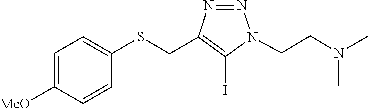 [SMILES: COc1ccc(SCc2nnn(CCN(C)C)c2I)cc1]

The procedure of Example 33 was followed starting from 4-methoxyphenyl propargyl sulfide (3.84 mmol, 648 mg) and 2-azido-N,N-dimethylethyl-1-amine (4.22 mmol, 482 mg). Yield 0.92 g (62%). Brown solid (Mp: 45° C.). ¹H NMR (400 MHz, CD₃OD) δ 7.26 (d, 2H), 6.84 (d, 2H), 4.50 (t, J=6.9 Hz, 2H), 4.01 (s, 2H), 3.79 (s, 3H), 2.80 (t, J=6.9 Hz, 2H), 2.32 (s, 6H). ¹³C NMR (101 MHz, CDCl₃) δ 159.3, 148.0, 134.7, 124.4, 114.1, 79.6, 57.9, 54.9, 48.2, 45.1, 31.2. IR (cm⁻¹): 2946, 2830, 2779, 1709, 1589, 1491, 1461, 1439, 1283, 1241,1173,1121,1101, 1021,858,821,788, 640,522,449.

### Example 52: (Compound 52)

### 1-[2-(N,N-dimethylamino)ethyl]-5-trithium-4-[(4-methoxyphenyl)thiomethyl]-1H-1,2,3-triazole

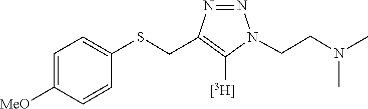

Tritium was obtained from RC Tritec AG. Tritium reactions were performed on an RC Tritec tritium manifold. 1-[2-(N,N-Dimethylamino)ethyl]-5-iodo-4-[(4-methoxyphenyl) thiomethyl]-1H-1,2,3-triazole, prepared according to Example 51, palladium on calcium carbonate and Et₃N (10 μL) in absolute ethanol were degassed by 3 freeze-pump-thaw cycles. The flask was filled with tritium gas, released from the uranium bed by heating it with a blowtorch, and stirred at room temperature for 4 hours. After this time the flask was frozen and the excess of tritium gas was blown off together with the solvent under nitrogen flow. The flask was removed from the bath and allowed to warm to room temperature. The reaction mixture was taken up in methanol and then passed through a syringe filter (0.45 μm PTFE filter). The solvent was evaporated under reduced pressure to yield a solid, which was purified via preparative HPLC using a Xbridge Prep C-18 column (5 μm OBD 19×100 mm) as stationary phase and, aqueous 0.1% ammonium formate (AFM)/MeCN as mobile phase. Chromatographic runs were performed under aqAFM/MeCN gradient conditions from 75:25 to 25:75 with a total chromatographic time of 30 min (flow rate=10 mL/min). The purified fractions were dried under vacuum and reconstituted in 10 mL of ethanol and stored at −20° C. Overall production time: 6 h. Isotopic incorporation: 87.1%. Molar activity at the end of the synthesis: 933.2 KBq/nmol. Radiochemical purity as determined by radio-HPLC >95%.

### C) Synthesis of Compounds of Formula (I) Wherein Z is —S(═O)—

### Example 53: (Compound 53)

### 1-[2-(N,N-dimethylamino)ethyl]-4-[(4-methoxyphenyl)sulfinylmethyl]-1H-1,2,3-triazole

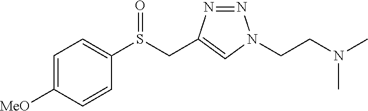 [SMILES: COc1ccc(S(=O)Cc2cn(CCN(C)C)nn2)cc1]

Phthaloyl peroxide (16.42 mmol, 2.69 g) was added to a solution of 1-[(2-(N,N-dimethylamino)ethyl]-4-[(4-methoxyphenyl)thiomethyl]-1H-1,2,3-triazole (13.7 mmol, 4.0 g) and trifluoroacetic acid (13.7 mmol, 1.10 mL) in CH₂Cl₂ (38 mL) and the mixture was stirred at room temperature for 30 minutes. The reaction mixture was basified by adding 7 N NH₃ in MeOH and evaporated under reduced pressure. The product was purified by column chromatography (silica gel; eluent: CH₂Cl₂/MeOH (7N NH₃) mixture). Yield 3.13 g (66%). White solid. ¹H NMR (400 MHz, CD₃OD) δ 7.81 (s, 1H), 7.49 (d, J=8.8 Hz, 2H), 7.95 (d, J=8.8 Hz, 2H), 4.51 (t, J=6.3 Hz, 2H), 4.30 (dd, J=13.7, 1.6 Hz, 2H), 3.86 (s, 3H), 2.82 (t, J=6.3 Hz, 2H), 2.31 (s, 6H). ¹³C NMR (101 MHz, CD₃OD) δ 164.1, 137.5, 133.5, 127.7, 126.7, 115.9, 59.5, 56.1, 53.6, 45.4. IR (cm⁻¹): 1592, 1495, 1459, 1303, 1249, 1172, 1086, 1022, 829, 524.

### Example 54: (Compound 54)

### 1-[3-(N,N-dimethylamino)propyl]-4-[(4-methoxyphenyl)sulfinylmethyl]-1H-1,2,3-triazole

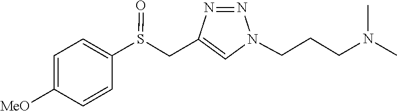 [SMILES: COc1ccc(S(=O)Cc2cn(CCCN(C)C)nn2)cc1]

The procedure of Example 53 was followed starting from 1-[(3-(N,N-dimethylamino)propyl]-4-[(4-methoxyphenyl)thiomethyl]-1H-1,2,3-triazole (0.47 mmol, 160 mg), TFA (0.47 mmol, 37 μL) and phthaloyl peroxide (0.70 mmol, 115 mg) in CH₂Cl₂ (3 mL). Yield 161 mg (77%). Oil. ¹H NMR (400 MHz, CD₃OD) δ 7.87 (s, 1H), 7.51 (d, J=2.1 Hz, 2H), 7.12 (d, J=4.7 Hz, 2H), 4.54 (t, J=6.8 Hz, 2H), 4.29 (dd, 2H), 3.88 (s, 3H), 3.13 (t, 2H), 2.88 (s, 6H), 2.36 (p, J=10.4, 5.0 Hz, 2H). ¹³C NMR (101 MHz, CD₃OD) δ 164.2, 138.1, 133.6, 132.3, 130.9, 130.1, 129.6, 129.1, 128.7, 127.6, 126.7, 116.0, 56.2, 56.1, 43.7, 26.5. IR (cm⁻¹): 3386, 2682, 2478, 1719, 1592, 1496, 1304, 1253, 1175, 1087, 1023, 833, 528.

### Example 55: (Compound 55)

### 1-[2-(N,N-diethylamino)ethyl]-4-[(4-methoxyphenyl)sulfinylmethyl]-1H-1,2,3-triazole

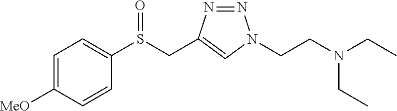 [SMILES: CCN(CC)CCn1cc(CS(=O)c2ccc(OC)cc2)nn1]

The procedure of Example 53 was followed starting from 1-[(2-(N,N-diethylamino)ethyl]-4-[(4-(methoxyphenyl)thiomethyl]-1H-1,2,3-triazole (0.34 mmol, 100 mg), TFA (0.34 mmol, 27 μL) and phthaloyl peroxide (0.50 mmol, 84 mg) in CH₂Cl₂ (2 mL). Yield 78 mg (67%). Oil. ¹H NMR (400 MHz, CD₃OD) δ 7.83 (s, 1H), 7.50 (d, J=8.8 Hz, 2H), 7.08 (d, J=8.9 Hz, 2H), 4.45 (t, J=6.6 Hz, 2H), 4.30 (dd, J=12.6, 1.8 Hz, 2H), 3.85 (s, 3H), 2.89 (t, J=6.6 Hz, 2H), 2.57 (q, J=7.2 Hz, 4H), 1.00 (t, J=7.2 Hz, 6H). ¹³C NMR (101 MHz, CD₃OD) δ 164.1, 137.4, 133.6, 127.7, 126.8, 115.9, 56.1, 53.6, 53.5, 49.4, 48.1, 11.9. IR (cm⁻¹): 2967, 1592, 1495, 1460, 1303, 1250, 1086, 1024, 829, 524.

### Example 56: (Compound 56)

### 1-[2-(piperidin-1-yl)ethyl]-4-[(3-trifluoromethylphenyl)sulfinylmethyl]-1H-1,2,3-triazole

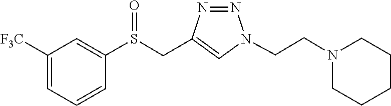 [SMILES: O=S(Cc1cn(CCN2CCCCC2)nn1)c1cccc(C(F)(F)F)c1]

The procedure of Example 53 was followed starting from 1-[(2-(piperidinyl)ethyl]-4-[(3-trifluoromethylphenyl)thiomethyl]-1H-1,2,3-triazole (0.74 mmol, 300 mg), TFA (0.74 mmol, 56 μL) and phthaloyl peroxide (1.11 mmol, 181 mg) in CH₂Cl₂ (2 mL). Yield 176 mg (62%). Oil. ¹H NMR (400 MHz, CDCl₃) δ 7.78 (s, 1H), 7.76-7.58 (m, 4H), 4.46 (td, J=6.5, 2.0 Hz, 2H), 4.23 (dd, J=12.9, 2.3 Hz, 2H), 2.77 (t, J=6.4 Hz, 2H), 2.46 (t, J=5.5 Hz, 4H), 1.59 (p, J=5.6 Hz, 4H), 1.49-1.40 (m, 2H).

### Example 57: (Compound 57)

### 4-[(4-isopropylphenyl)sulfinylmethyl]-1-[2-(4-morpholinyl)ethyl]-1H,1,2,3-triazole

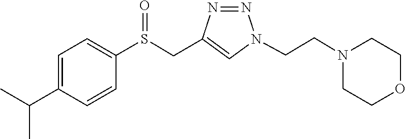 [SMILES: CC(C)c1ccc(S(=O)Cc2cn(CCN3CCOCC3)nn2)cc1]

The procedure of Example 53 was followed starting from 4-[(4-isopropyl phenyl)thiomethyl]-1-[2-(4-morpholinyl)ethyl]-1H-1,2,3-triazole (1.47 mmol, 507 mg), TFA (1.47 mmol, 112 μL) and phthaloyl peroxide (1.62 mmol, 265 mg) in CH₂Cl₂ (5 mL). Yield 340 mg (64%). White solid (Mp: 131° C.). ¹H NMR (400 MHz, CDCl₃) δ 7.70 (s, 1H), 7.43 (d, J=8.4 Hz, 2H), 7.31 (d, J=8.0 Hz, 2H), 4.55-4.33 (m, 2H), 4.13 (dd, J=12.6, 1.8 Hz,), 3.65 (t, J=4.6 Hz, 4H), 2.92 (hept, J=6.9 Hz, 1H), 2.77 (t, J=6.3 Hz, 2H), 2.51-2.39 (m, 4H), 1.23 (d, J=6.9 Hz, 6H).

### Example 58: (Compound 58)

### 4-[(2,6-dimethylphenyl)sulfinylmethyl]-1-[2-(piperidin-1-yl)ethyl]-1H-1,2,3-triazole

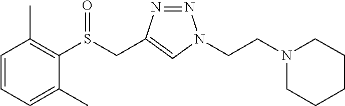 [SMILES: Cc1cccc(C)c1S(=O)Cc1cn(CCN2CCCCC2)nn1]

The procedure of Example 53 was followed starting from 4-[(2,6-dimethylphenyl)thiomethyl]-1-[(2-(piperidinyl)ethyl]-1H-1,2,3-triazole (1.64 mmol, 543 mg), TFA (1.64 mmol, 126 μL) and phthaloyl peroxide (1.81 mmol, 296 mg) in CH₂Cl₂ (5 mL). Yield 364 mg (64%). White solid (Mp: 84-85° C.). ¹H NMR (400 MHz, CDCl₃) δ 7.54 (s, 1H), 7.32-7.21 (m, 1H), 7.04 (d, J=7.6 Hz, 2H), 4.62-4.32 (m, 4H), 2.77-2.65 (m, 2H), 2.49 (s, 6H), 2.46-2.38 (m, 4H), 1.57 (p, J=5.5 Hz, 4H), 1.51-1.40 (m, 2H).

### Example 59: (Compound 59)

### 1-[2-(4-morpholinyl)ethyl]-4-(phenylsulfinylmethyl)-1H,1,2,3-triazole

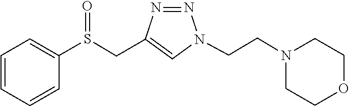 [SMILES: O=S(Cc1cn(CCN2CCOCC2)nn1)c1ccccc1]

The procedure of Example 53 was followed starting from 1-[2-(4-morpholinyl) ethyl]-4-[phenylthiomethyl]-1H-1,2,3-triazole (0.86 mmol, 260 g), TFA (0.86 mmol, 66 μL) and phthaloyl peroxide (0.95 mmol, 155 mg) in CH₂Cl₂ (3 mL). Yield 167 g (61%). White solid (Mp: 134° C.). ¹H NMR (400 MHz, CDCl₃) δ 7.65 (s, 1H), 7.54-7.38 (m, 5H), 4.47-4.31 (m, 2H), 4.13 (dd, J=13.1, 1.9 Hz, 2H), 3.62 (t, J=4.6 Hz, 4H), 2.73 (t, J=6.2 Hz, 2H), 2.48-2.36 (m, 4H).

### Example 60: (Compound 60)

### 4-[(4-methoxyphenyl)sulfinylmethyl]-1-[2-(piperidin-1-yl)ethyl]-1H-1,2,3-triazole

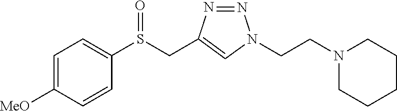 [SMILES: COc1ccc(S(=O)Cc2cn(CCN3CCCCC3)nn2)cc1]

The procedure of Example 53 was followed starting from 4-[4-methoxyphenylthiomethyl]-1-[2-(piperidin-1-yl)ethyl]-1H-1,2,3-triazole (1.50 mmol, 500 g), TFA (1.50 mmol, 115 μL) and phthaloyl peroxide (2.26 mmol, 370 mg) in CH₂Cl₂ (4 mL). Yield 325 g (62%). Oil. ¹H NMR (400 MHz, CDCl₃) δ 7.64 (s, 1H), 7.38 (d, J=8.9 Hz, 2H), 6.92 (d, J=9.0 Hz, 2H), 4.49-4.27 (m, 2H), 4.10 (dd, J=12.9, 1.7 Hz, 2H), 3.78 (s, 3H), 2.70 (t, J=6.5 Hz, 2H), 2.49-2.30 (m, 4H), 1.52 (p, J=5.6 Hz, 4H), 1.39 (q, J=5.9 Hz, 2H).

### Example 61: (Compound 61)

### 1-[1-(R)-benzyl-2-(piperidin-1-yl)ethyl]-4-[(3-trifluoromethylphenyl)sulfinylmethyl]-1H-1,2,3-triazole

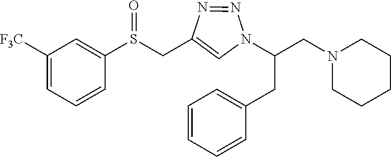 [SMILES: O=S(Cc1cn(C(Cc2ccccc2)CN2CCCCC2)nn1)c1cccc(C(F)(F)F)c1]

The procedure of Example 53 was followed starting from 1-[1-(R)-benzyl-2-(piperidin-1-yl)ethyl]-4-[(3-trifluoromethylphenyl)sulfinylmethyl]-1H-1,2,3-triazole (0.16 mmol, 80 mg), TFA (0.74 mmol, 12 μL) and phthaloyl peroxide (0.24 mmol, 40 mg) in CH₂Cl₂ (0.5 mL). Yield 50 mg (65%). Oil. ¹H NMR (400 MHz, CDCl₃) δ 7.93-7.67 (m, 2H), 7.63-7.44 (m, 2H), 7.41-7.15 (m, 4H), 7.10-6.94 (m, 2H), 4.83 (ddt, J=12.7, 9.5, 4.7 Hz, 1H), 4.31-4.07 (m, 3H), 3.25 (dtt, J=23.2, 9.1, 5.0 Hz, 2H), 2.92-2.78 (m, 2H), 2.43 (dt, J=11.1, 5.2 Hz, 2H), 2.34 (dq, J=10.4, 5.0 Hz, 2H), 1.53 (dd, J=8.8, 3.2 Hz, 4H), 1.41 (d, J=6.2 Hz, 2H).

### Example 62: (Compound 62)

### 1-[3-(N,N-diethylamino)-2-methyl-propyl]-4-[(4-methoxyphenyl)sulfinylmethyl]-1H-1,2,3-triazole

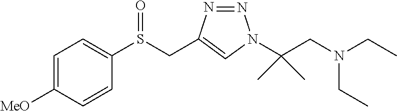 [SMILES: CCN(CC)CC(C)(C)n1cc(CS(=O)c2ccc(OC)cc2)nn1]

The procedure of Example 53 was followed starting from 1-[3-(N,N-diethylamino)-2-methyl-propyl]-4-[(4-methoxyphenyl)thiomethyl]-1H-1,2,3-triazole (0.21 mmol, 71 mg), TFA (0.21 mmol, 16 μL) and phthaloyl peroxide (0.31 mmol, 50 mg) in CH₂Cl₂ (1 mL). Yield 57 mg (75%). Oil. ¹H NMR (400 MHz, CDCl₃) δ 7.65 (s, 1H), 7.51 (d, J=8.8 Hz, 1H), 7.02 (d, J=8.8 Hz, 1H), 4.19 (dd, J=13.5, 1.0 Hz, 2H), 3.87 (s, 3H), 2.73 (s, 2H), 2.28 (q, J=7.4 Hz, 2H), 1.65 (s, 3H), 1.64 (s, 3H), 0.86 (t, J=7.1 Hz, 5H).

### Example 63: (Compound 63)

### 1-[2-(N,N-dimethylamino)ethyl]-4-[(4-hydroxyphenyl)sulfinylmethyl]-1H-1,2,3-triazole

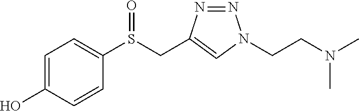 [SMILES: CN(C)CCn1cc(CS(=O)c2ccc(O)cc2)nn1]

The procedure of Example 53 was followed starting from 4-[4-hydroxyhenylthiomethyl]-1-[(2-(N,N-dimethylamino)ethyl]-1H-1,2,3-triazole (0.26 mmol, 71 g), TFA (0.26 mmol, 20 μL) and phthaloyl peroxide (0.31 mmol, 50 mg) in CH₂Cl₂ (0.7 mL). Yield 59 mg (78%). White solid. ¹H NMR (400 MHz, CD₃OD) δ 7.89 (s, 1H), 7.41 (d, J=8.7 Hz, 2H), 6.95 (d, J=8.6 Hz, 2H), 4.78 (t, J=6.4 Hz, 2H), 4.28 (dd, J=12.5, 2.0 Hz, 2H), 3.44 (t, J=6.5 Hz, 2H), 2.74 (s, 6H).

### Example 64: (Compound 64)

### 1-(2-aminoethyl)-4-[(4-methoxyphenyl)sulfinylmethyl]-1H-1,2,3-triazole

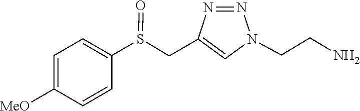 [SMILES: COc1ccc(S(=O)Cc2cn(CCN)nn2)cc1]

To a solution of 1-(2-aminoethyl)-4-[(4-methoxyphenyl)thiomethyl]-1H-1,2,3-triazole (2.33 mmol, 700 mg) in dry CH₂Cl₂ (14 mL) under N₂, di-tert-butyl dicarbonate (2.44 mmol, 533 mg), DIPEA (4.66 mmol, 798 μL) and 4-(N,N-dimethylamino)pyridine (catalyst, 20 mg) were added and the reaction mixture was stirred at room temperature overnight. The N-Boc intermediate product was purified by column chromatography (silica gel; eluent: CH₂Cl₂/MeOH 95:5). Yield: 460 mg (54%). ¹H NMR (400 MHz, CDCl₃) δ 7.32 (d, J=8.8 Hz, 2H), 7.28 (s, 1H), 6.83 (d, J=8.7 Hz, 2H), 4.18-4.07 (m, 4H), 3.80 (s, 3H), 3.62-3.52 (m, 2H), 1.45 (s, 9H). The procedure of Example 53 was followed to oxidize 1-(2-tert-butoxycarbonylaminoethyl)-4-[(4-methoxyphenyl) thiomethyl]-1H-1,2,3-triazole (0.27 mmol, 100 mg) using phthaloyl peroxide (0.41 mmol, 67.5 mg) in CH₂Cl₂ (1.8 mL). Yield: 56 mg (78%). ¹H NMR (400 MHz, CDCl₃) δ 7.57 (s, 1H), 7.43 (d, J=8.8 Hz, 2H), 6.98 (d, J=8.8 Hz, 2H), 4.45 (t, J=5.7 Hz, 2H), 4.23-4.08 (m, 2H), 3.84 (s, 3H), 3.65-3.55 (m, 2H), 1.43 (s, 9H). Trifluoroacetic acid (0.82 mL) was added at 0° C. to a solution of 1-(2-tert-butoxycarbonylaminoethyl)-4-[(4-methoxyphenyl)sulfinylmethyl]-1H-1,2,3-triazole (0.15 mmol, 56 mg) in dry CH₂Cl₂ (2.4 mL) and the reaction mixture was stirred at room temperature for 3 hours. The product was purified by column chromatography (silica gel; eluent: CH₂Cl₂/MeOH (7N NH₃) 95:5). Yield 39 mg (94%). Oil. ¹H NMR (400 MHz, CD₃OD) δ 7.76 (s, 1H), 7.49 (d, J=8.9 Hz, 2H), 7.10 (d, J=8.9 Hz, 2H), 4.43 (t, J=6.1 Hz, 2H), 4.37-4.25 (m, 2H), 3.88 (s, 3H), 3.09 (t, J=6.1 Hz, 2H).

### Example 65: (Compound 65)

### 1-[2-(N-methylamino)-ethyl]-4-[(4-methoxyphenyl)sulfinylmethyl]-1H-1,2,3-triazole

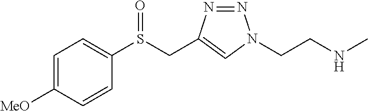 [SMILES: CNCCn1cc(CS(=O)c2ccc(OC)cc2)nn1]

The procedure of Example 64 was followed starting from 1-[2-(N-methylamino)-ethyl]-4-[(4-methoxyphenyl)thiomethyl]-1H-1,2,3-triazole (1.39 mmol, 388 mg) to provide the intermediate 1-[2-(N-tert-butoxycarbonyl-N-methylamino)-ethyl]-4-[(4-methoxyphenyl) sulfinylmethyl]-1H-1,2,3-triazole. Yield: 164 mg (98%). ¹H NMR (400 MHz, CDCl₃) δ 7.49 (s, 1H), 7.44-7.38 (m, 2H), 7.00-6.85 (m, 2H), 4.52-4.37 (m, 2H), 4.20-4.04 (m, 2H), 3.80 (s, 3H), 3.62 (td, J=6.0, 2.0 Hz, 2H), 2.74-2.60 (m, 3H), 1.38 (s, 9H). This compound (0.41 mmol, 164 mg) was N-deprotected with trifluoroacetic acid (2.30 mL) in dry CH₂Cl₂ (6.5 mL) to provide the product. Yield 115 (95%). Oil. ¹H NMR (400 MHz, CD₃OD) δ 7.77 (s, 1H), 7.50 (d, J=8.9 Hz, 2H), 7.10 (d, J=8.9 Hz, 2H), 4.50 (t, J=6.2 Hz, 2H), 4.41-4.25 (m, 2H), 3.88 (s, 3H), 3.03 (t, J=6.2 Hz, 2H), 2.41 (s, 3H).

### Example 66: (Compound 66)

### 1-[2-(N,N-dimethyl)-N-oxideaminoethyl]-4-[(4-methoxyphenyl)sulfinylmethyl]-1H-1,2,3-triazole

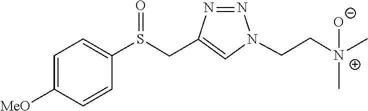 [SMILES: COc1ccc(S(=O)Cc2cn(CC[N+](C)(C)[O-])nn2)cc1]

The procedure of Example 34 was followed starting from 1-[2-(N,N-dimethylamino)-ethyl]-4-[(4-methoxyphenyl)thiomethyl]-1H-1,2,3-triazole (1.00 mmol, 292 mg) and 33% hydrogen peroxide (1.5 mL). Yield 314 mg (97%). Oil.

¹H NMR (400 MHz, MeOD) δ 7.95 (d, J=1.4 Hz, 1H), 7.49 (d, J=8.9 Hz, 2H), 7.09 (d, J=8.9 Hz, 2H), 5.02 (t, J=6.4 Hz, 2H), 4.34 (q, J=13.9 Hz, 2H), 3.98 (t, J=6.4 Hz, 2H), 3.87 (s, 3H), 3.28 (s, 6H).

### D) Synthesis of Compounds of Formula (I) Wherein Z is —S(═O)₂—

### Example 67: (Compound 67)

### 1-[2-(N,N-dimethylamino)ethyl]-4-[(4-methoxyphenyl)sulfonylmethyl]-1H-1,2,3-triazole

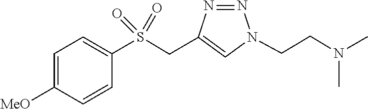 [SMILES: COc1ccc(S(=O)(=O)Cc2cn(CCN(C)C)nn2)cc1]

Oxone (1.28 mmol, 788 mg) was added to a solution of 1-[(2-N,N-dimethylamino)ethyl]-4-[(4-methoxyphenyl)thiomethyl]-1H-1,2,3-triazole (0.86 mmol, 250 mg) in H₂O/MeCN 50:50 (5 mL) and the reaction mixture was stirred at room temperature for 1 hour. Upon basification by adding solid Na₂CO₃, the product was extracted with EtOAc (3×5 mL), the organic combined layers were dried (Na₂SO₄) and the solvent was evaporated at reduced pressure. The crude product was purified by column chromatography (silica gel; eluent: EtOAc/Hex or CH₂Cl₂/MeOH mixture). Yield 167 mg (60%). Oil. ¹H NMR (400 MHz, CDCl₃) δ 7.88 (s, 1H), 7.61 (d, J=7.7 Hz, 2H), 6.92 (d, J=8.9 Hz, 2H), 4.51, (s, 2H), 4.43 (t, J=6.2 Hz, 2H), 3.84 (s, 3H), 2.73 (t, J=6.2 Hz, 2H), 2.27 (s, 6H). ¹³C NMR (101 MHz, CDCl₃) δ 163.8, 135.6, 130.4, 129.2, 125.0, 114.2, 58.5, 55.5, 54.1, 48.2, 45.2.

### Example 68: (Compound 68)

### 1-[2-(N,N-diethylamino)ethyl]-4-[(4-bromophenyl)sulfonylmethyl]-1H-1,2,3-triazole

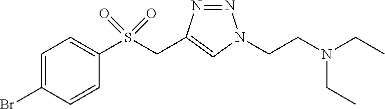 [SMILES: CCN(CC)CCn1cc(CS(=O)(=O)c2ccc(Br)cc2)nn1]

The procedure of Example 67 was followed starting from 4-[(4-bromophenyl) thiomethyl]-1-[(2-N,N-diethylamino)ethyl]-1H-1,2,3-triazole (2.00 mmol, 739 mg) and oxone (3.00 mmol, 1.84 g). Yield 545 mg (68%). White solid (Mp: 88° C.). ¹H NMR (400 MHz, CDCl₃) δ 7.93 (s, 1H), 7.65 (d, J=8.6 Hz, 2H), 7.57 (d, J=8.6 Hz, 2H), 4.56 (s, 2H), 4.43 (t, J=6.1 Hz, 2H), 2.89 (d, J=6.1 Hz, 2H), 2.58 (q, J=7.1 Hz, 4H), 1.00 (t, J=7.1 Hz, 6H).

### Example 69: (Compound 69)

### 1-[2-(N,N-dimethylamino)ethyl]-4-[(3,4-dimethoxyphenyl)sulfonylmethyl]-1H-1,2,3-triazole

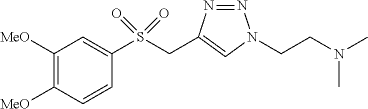 [SMILES: COc1ccc(S(=O)(=O)Cc2cn(CCN(C)C)nn2)cc1OC]

The procedure of Example 67 was followed starting from 4-[(3,4-dimethoxyphenyl)thiomethyl]-1-[(2-N,N-dimethylamino)ethyl]-1H-1,2,3-triazole (2.00 mmol, 645 mg) and oxone (3.00 mmol, 1.84 g). Yield 545 mg (77%). White solid (Mp: 152° C.). ¹H NMR (400 MHz, CDCl₃) δ 7.91 (s, 1H), 7.31 (dd, J=8.5, 2.1 Hz, 1H), 7.17 (d, J=2.1 Hz, 1H), 6.91 (d, J=8.5 Hz, 1H), 4.54 (s, 2H), 4.46 (t, J=6.2 Hz, 2H), 3.94 (s, 3H), 3.88 (s, 3H), 2.76 (t, J=6.2 Hz, 2H), 2.29 (s, 6H).

### Example 70: (Compound 70)

### 4-[(4-isopropylphenyl)sulfonylmethyl]-1-[2-(4-morpholinyl)ethyl]-1H,1,2,3-triazole

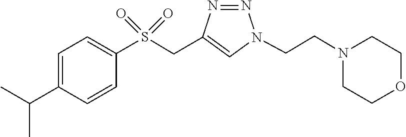 [SMILES: CC(C)c1ccc(S(=O)(=O)Cc2cn(CCN3CCOCC3)nn2)cc1]

The procedure of Example 67 was followed starting from 4-[(4-isopropylphenyl)thiomethyl]-1-[2-(4-morpholinyl)ethyl]-1H,1,2,3-triazole (2.00 mmol, 693 mg) and oxone (3.00 mmol, 1.84 g). Yield 401 mg (53%). Oil. ¹H NMR (400 MHz, CDCl₃) δ 7.93 (s, 1H), 7.63 (d, J=8.4 Hz, 2H), 7.32 (d, J=8.4 Hz, 2H), 4.53 (s, 2H), 4.46 (t, J=6.1 Hz, 2H), 3.74-3.61 (m, 4H), 2.95 (hept, J=6.9 Hz, 1H), 2.80 (t, J=6.2 Hz, 2H), 2.53-2.43 (m, 4H), 1.24 (d, J=6.9 Hz, 6H).

### Example 71: (Compound 71)

### 4-(phenylsulfonylmethyl)-1-[2-(4-morpholinyl)ethyl]-1H,1,2,3-triazole

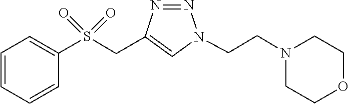 [SMILES: O=S(=O)(Cc1cn(CCN2CCOCC2)nn1)c1ccccc1]

The procedure of Example 67 was followed starting from 4-(phenylthiomethyl)-1-[2-(4-morpholinyl)ethyl]-1H,1,2,3-triazole (0.85 mmol, 260 mg) and oxone (1.28 mmol, 788 mg) in H₂O (4 mL). Yield. 108 mg (38%). Oil. ¹H NMR (400 MHz, CDCl₃) δ 7.93 (s, 1H), 7.77-7.72 (m, 2H), 7.66 (td, J=7.2, 1.3 Hz, 1H), 7.56-7.50 (m, 2H), 4.58 (s, 2H), 4.50 (t, J=6.1 Hz, 2H), 3.77-3.71 (m, 4H), 2.84 (t, J=6.1 Hz, 2H), 2.54-2.51 (m, 4H).

### Example 72: (Compound 72)

### 1-[2-(N,N-dimethylamino)ethyl]-4-[(3-trifluoromethylphenyl)sulfonylmethyl]-1H-1,2,3-triazole

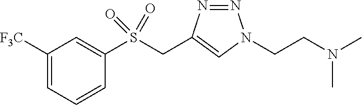 [SMILES: CN(C)CCn1cc(CS(=O)(=O)c2cccc(C(F)(F)F)c2)nn1]

The procedure of Example 67 was followed starting from 1-[2-(N,N-dimethylamino)ethyl]-4-[(3-trifluoromethylphenyl)thiomethyl]-1H-1,2,3-triazole (0.76 mmol, 250 mg) and oxone (1.13 mmol, 698 mg). Yield 85 mg (31%). ¹H NMR (400 MHz, CD₃OD) δ 8.10-7.97 (m, 4H), 7.82 (t, J=7.9 Hz, 1H), 4.77 (s, 2H), 4.53 (t, J=6.5 Hz, 2H), 2.81 (t, J=6.5 Hz, 2H), 2.29 (s, 6H).

### Example 73: (Compound 73)

### 1-(2-aminoethyl)-4-[(4-methoxyphenyl)sulfonylmethyl]-1H-1,2,3-triazole

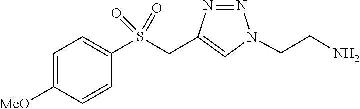 [SMILES: COc1ccc(S(=O)(=O)Cc2cn(CCN)nn2)cc1]

The procedure of Example 67 was followed to oxidize 1-(2-tert-butoxycarbonylaminoethyl)-4-[(4-methoxyphenyl)thiomethyl]-1H-1,2,3-triazole (0.27 mmol, 100 mg) with oxone (0.41 mmol, 253 mg) to the corresponding sulfone. Yield: 105 mg (97%). ¹H NMR (400 MHz, CDCl₃) δ 7.78 (s, 1H), 7.66 (d, J=8.9 Hz, 2H), 6.98 (d, J=8.9 Hz, 2H), 4.54 (s, 2H), 4.51 (t, J=5.7 Hz, 2H), 3.89 (s, 3H), 3.65 (q, J=5.9 Hz, 2H), 1.47 (s, 9H). To a solution of 1-(2-tert-butoxycarbonylaminoethyl)-4-[(4-methoxyphenyl)sulfonylmethyl]-1H-1,2,3-triazole (0.27 mmol, 105 mg) in dry CH₂Cl₂ (5 mL), trifluoroacetic acid (1.6 mL) was added at 0° C. Then, the reaction mixture was stirred at room temperature for 3 hours, solvents were evaporated and the product was purified by column chromatography (silica gel; eluent: CH₂Cl₂/MeOH (7N NH₃) 95:5). Yield 78 mg (97%). Oil. ¹H NMR (400 MHz, CD₃OD) δ 7.94 (s, 1H), 7.69 (d, J=8.9 Hz, 2H), 7.09 (d, J=8.9 Hz, 2H), 4.64 (s, 2H), 4.45 (t, J=6.2 Hz, 2H), 3.90 (s, 3H), 3.09 (t, J=6.2 Hz, 2H).

### Example 74: (Compound 74)

### 1-[2-(N-methylamino)-ethyl]-4-[(4-methoxyphenyl)sulfonylmethyl]-1H-1,2,3-triazole

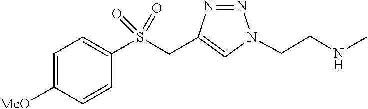 [SMILES: CNCCn1cc(CS(=O)(=O)c2ccc(OC)cc2)nn1]

The procedure of Example 73 was followed starting from 1-[2-(N-methylamino)-ethyl]-4-[(4-methoxyphenyl)thiomethyl]-1H-1,2,3-triazole (1.39 mmol, 388 mg) The N-Boc product was purified by column chromatography (silica gel; eluent: CH₂Cl₂/MeOH 95:5). Yield: 500 mg (95%). ¹H NMR (400 MHz, CDCl₃) δ 7.32 (d, J=8.7 Hz, 2H), 7.21 (s, 1H), 6.89-6.76 (m, 2H), 4.53-4.37 (m, 2H), 4.13 (s, 2H), 3.79 (s, 3H), 3.64 (t, J=6.1 Hz, 2H), 2.72-2.56 (m, 3H), 1.44 (s, 9H). The procedure of Example 64 was followed to oxidize the N-Boc protected sulfide (0.36 mmol, 135 mg) with oxone (0.54 mmol, 329 mg) to the corresponding sulfone. Yield: 127 mg (87%). ¹H NMR (400 MHz, CDCl₃) δ 7.75 (s, 1H), 7.67 (d, J=8.9 Hz, 2H), 7.02-6.93 (m, 2H), 4.62-4.48 (m, 4H), 3.89 (s, 3H), 3.71 (t, J=6.1 Hz, 2H), 2.79-2.64 (m, 3H), 1.47 (s, 9H). N-deprotection of the N-Boc-N-methylamino intermediate following the procedure of Example 73 afforded the product. Yield 106 mg (95%). Oil. ¹H NMR (400 MHz, CD₃OD) δ 7.95 (s, 1H), 7.69 (d, J=8.9 Hz, 2H), 7.09 (d, J=9.0 Hz, 2H), 4.85 (s, 2H), 4.63 (s, 2H), 4.52 (t, J=6.2 Hz, 2H), 3.90 (s, 3H), 3.04 (t, J=6.2 Hz, 2H), 2.41 (s, 3H).

### Example 75. Assay of Retinal Biodistribution after Topical Ocular Administration in 4 Mammalian Species

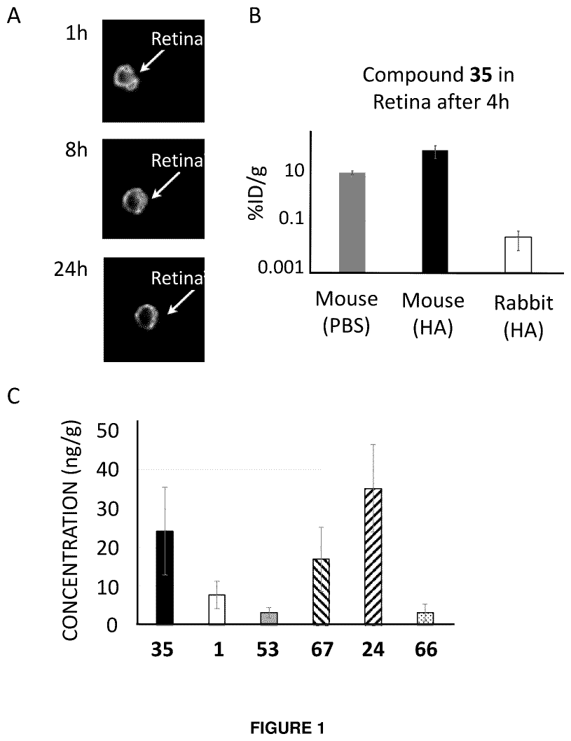

In order to test the bioavailability of the compounds 1, 24, 35, 53, 66 and 67 to the retina, the following species we used: mouse, rat, rabbit and swine. Animals were treated under anesthesia. In vivo autoradiography imaging was conducted at 1, 8 and 24 hours following the ocular instillation of compound [³H]-35 (5 μL/eye) as a phosphate buffered saline (PBS) 20 mM solution under carrier added conditions (ca. 7 mBq/eye) on Sprague-Dawley rats (n=4 for each time point). After the enucleation, the eye was incubated firstly in an aqueous solution at 4% of paraformaldehyde for 4 hours, then in aqueous solution at 10% and 20% of sucrose for 2 hours each and finally in aqueous solution at 30% of sucrose for one week. All the incubations were at 4° C. The samples were fixed in OCT (optimal cutting temperature polymer) and fine 20-μm sections of eye were cut using a Leyca cryostat (Leica CM3050 S, Germany) at −24° C. The slices (20 μm thick) were disposed on glass slides (Superfrost Plus, Labolan). Autoradiography was performed in Beta Imager 2000 system (Biospace Lab, France), for 1 h on four consecutive slices of the central part of the organ where all the parts are visible (FIG. 1 A).

Ocular instillation with PBS solutions containing 0.2% hyaluronic acid (HA) and the selected compounds of the invention were administered: 17 μg/eye (3 μL of 20 mM solution per eye) in mouse; 65 μg/eye (10 μL of 17 mM solution per eye) in rat; and 559 μg/eye (50 μL of 40 mM solution per eye) in swine. Animals were sacrificed after 4 hours (mouse, rabbit and swine) or after 24 hours (rats) of treatment to determinate the availability of the compounds in the retina. Retinas were mechanically homogenized in physiological serum (9% NaCl) and processed to analyze the compounds availability by HPLC-MS. FIG. 1B shows a higher delivery percentage of compound 35 to the mouse retina when formulated with hyaluronic acid. FIG. 1C shows the retinal biodistribution results of 5 compounds after topical ocular administration in mice, rats, rabbits and pigs.

### Example 76. In Vivo Efficacy Assay in Rd10 Mouse Model of Retinitis Pigmentosa, as Measured by Electrophysiological Analysis

rd10 mice were treated by ocular instillation with phosphate buffered saline (PBS) containing the compounds of the invention (1, 35, 50, 56 and 70) for 11 days (from P14 to P24). Animals were dark adapted for 8 hours at P25 and full-field flash electroretinographic (ERG) responses were recorded with the retina illuminated with a LED-driven Ganzfeld dome. A series of light flashes of increasing intensity, from 0.001 to 10 cd s/m², were averaged both in scotopic (dark-adapted) and photopic (light adapted) conditions. Photopic cone responses were recorded following 5 min of light adaptation with a background white light (30 cd/m²). Light intensity was controlled for each animal group (Mavo-Monitor USB, Gossen, Germany). The stimulation protocols were designed according to the International Society for Clinical Electrophysiology of Vision. Briefly, mice were anesthetized with intraperitoneal administration of a saline solution containing ketamine (95 mg/kg) and xylazine (5 mg/kg) and kept on a 37° C. heating pad. Ocular instillation of 1% tropicamide was applied to induce mydriasis. ERG responses were recorded from both eyes in response to a Ganzfeld stimulator under light flashes. A total of 4 to 64 consecutive stimuli were averaged, with an interval between light flashes in scotopic conditions of 10 s for dim flashes and of up to 60 s for the highest intensity for every light intensity used. For photopic conditions, a one-second interval between light flashes was used. ERG signals were amplified, and band filtered between 0.3 and 1000 Hz (CP511 AC amplifier; Grass Instruments, Quincy, MA, USA). Electrical signals were digitized at 20 kHz with a power laboratory data acquisition board (AD Instruments, Chalgrove, UK).

ERGs were performed using an electrode fixed to a corneal lens (Burian-Allen electrode; Hansen Ophthalmic Development Laboratory, Coralville, IA, USA) and a reference electrode fixed to the mouth, with a ground electrode placed in the tail. Rod-mediated responses were recorded from light flashes ranging from 0.001 to 10 cd s/m² with dark-adapted mice. Mixed rod- and cone-mediated signals were recorded in response to −1.5 to 1.5 log cd s/m² light flashes. Oscillatory potential (OP) was isolated using 11 cd s/m² white flashes, with a recording frequency ranging between 100 and 10,000 Hz. For recording of cone-mediated responses, we applied light flashes ranging from 0.5 to 2 log cd s/m² on a rod-saturating background of 30 cd s/m². Amplitudes of the a-wave and b-wave were averaged, using data provided by a researcher with no information regarding the experimental condition of mice.

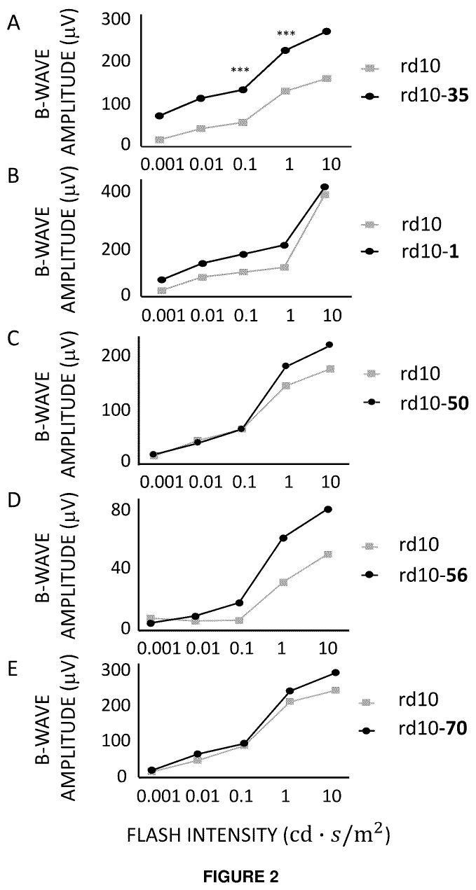

FIG. 2 shows an increased retinal function of rd10 mice treated with the compounds of the invention (1, 35, 50, 56 and 70; 3 μL of 20 mM solution per eye) as measured by ERG activity after stimulation with light at different intensities (0.001; 0.01; 0.1; 1 and 10 cd s/m²) in dark-adapted mice (scotopic conditions). Data correspond to a pool of 3 independent experiments (N=3) with 4-5 mice per experimental group. Data are expressed as mean+/−standard deviation. Unpaired t-test was used for statistical analysis. All compounds tested showed different degrees of ERG activity increase that can be summarized as follows:
The b-wave amplitude was significantly increased in mice treated with the compounds in the dark-adapted ERG at flash intensities ranging from 0.001 to 10 cd s/m².
Compounds improved rod function by increasing ERG amplitude in scotopic conditions in up to 42 and 44% at 0.002 and 0.02 cd s/m² light intensities, respectively, compared to rd10 non-treated group (***p=0.0014; *p=0.0194). This accounts for an average recovery score of 32% compared to wild-type mice.
Scotopic oscillatory potential at maximal intensities, which is related to the function of amacrine and Muller cells, was significantly increased in treated rd 10 mice by up to 81% (*p=0.039), with a recovery score of 25%.
Some compounds (e.g. compound 35) significantly improved cone function by increasing ERG amplitude by 66% at 0.2 cd s/m² light intensity (vs. rd10 control group, *p=0.0113) and exhibited 78% recovery score vs. WT.

### Example 77. In Vivo Efficacy Assay in Rd10 Mouse Model of Retinitis Pigmentosa, as Measured by Histological Analysis

In this experiment, rd10 mice were treated by ocular instillation with phosphate buffered saline (PBS) solutions containing the compounds of the invention (1, 35, 53, and 36; 3 μL of 20 mM solution per eye) for 11 days (from P14 to P24). At P25 eyeballs were enucleated and fixed with 4% paraformaldehyde (in PBS) for 3 hours. The anterior segment and lens were dissected, and the eyecups were incubated in 30% sucrose (in PBS) for 12 h at 4° C. Cryosections of 12 μM were obtained and stained with DAPI. The sections were analyzed by fluorescent microscopy, and the number of nuclei in the outer nuclear layer, that contains the nuclei of photoreceptors, was compared.

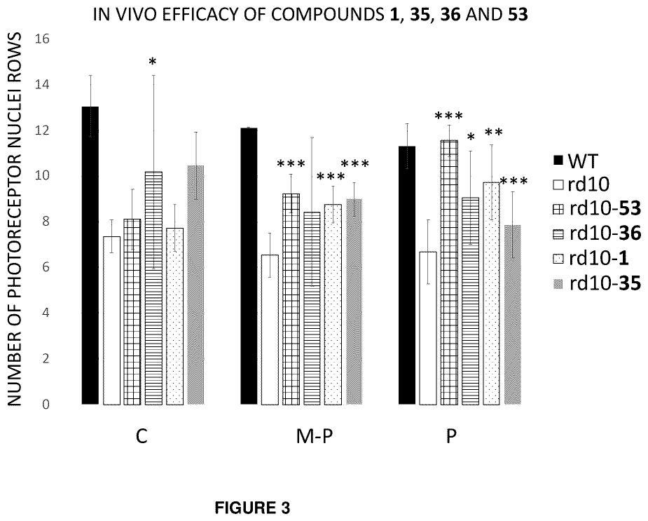

FIG. 3 shows an improved number of photoreceptor nuclei in the retinas of rd10 mice treated with 4 different compounds, with respect to untreated mice at central (C), mid-periphery (M-P) and peripheral (P) retina.

### Example 78. Morphometric Analysis of the Retina

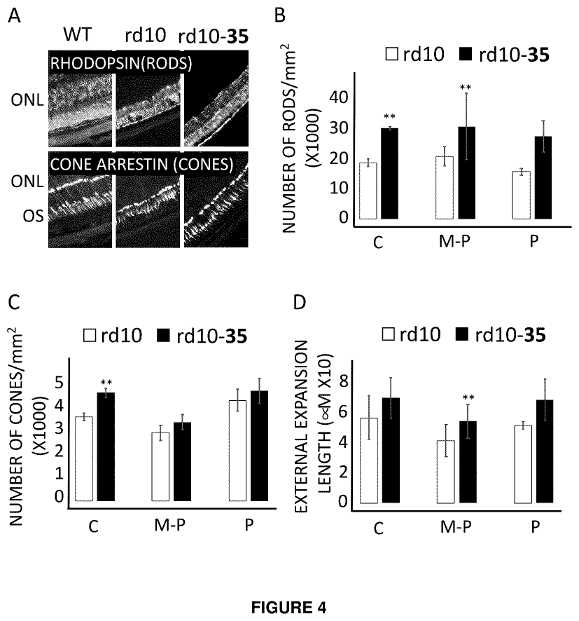

A detailed morphometric analysis, based on immunohistochemistry, was performed. For this purpose, rd10 mice were treated by ocular instillation with phosphate buffered saline (PBS) containing compound 35 (3 μL of 20 mM solution per eye) for 11 days (from P14 to P24). At P25 eyeballs were enucleated and fixed with 4% paraformaldehyde (in PBS) for 3 hours. The anterior segment and lens were dissected, and the eyecups were incubated in 30% sucrose (in PBS) for 12 h at 4° C. Cryosection with DAPI. ocular cryosections were stained with DAPI, rhodopsin and cone arrestin for immunohistochemical analysis. Briefly, cryosections were incubated with antibodies against rhodopsin (Millipore, MABN15) and cone arrestin (Millipore, AB15282) both at 1:400 for 8 h at 4° C. and buffered with secondary antibodies both at 1:400 and DAPI (Sigma, D89542) 1:1000 for 1 hour. The sections were analyzed by fluorescent microscopy. As shown in FIG. 4, a significant preservation of photoreceptors in the retinas of rd10 mice treated with compound 35, as measured by the number of rods (B) and cones (C) per square mm, or the rod outer and inner segment (D) was observed.

### Example 79. In Vivo Efficacy Assay in Rd10 Mouse Model of Retinitis Pigmentosa as Measured by Optokinetic Response Based on a Water Maze

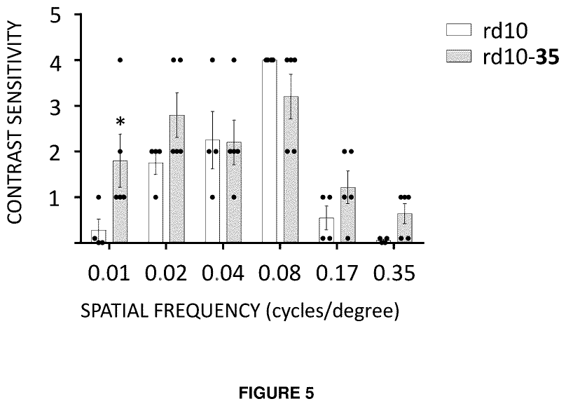

rd10 mice were treated by ocular instillation with phosphate buffered saline (PBS) containing the compound 35 of the invention for 16 days (from P14 to P29). To study optokinetic (OKT) response, we conducted an OKT test in combination with a modified Morris water maze using 9 rd10 mice at p30: 5 treated with compound 35 and 4 untreated. Animals were adapted to the experimental conditions for 1 week before behavioral testing. Mice were trained in a square pool (70×40 cm) filled with water (24-26-C), with all sides opaque, except the frontal side, where a screen was placed. The screen was divided vertically into 2 equal parts. A pattern of moving vertical bars was presented on one half of the screen, with the other half turned off. The direction of the bars was random: 50% of the time moving to the right and 50% of the time moving to the left. The bar velocity was constant throughout the process. Mice were trained for one week to associate the area of the screen with moving bars with the presence of a submerged platform, not visible to the mice under the dim light conditions, that was placed right next to that area of the screen. The platform allowed the mice to escape from the water. The experimental procedure was conducted over 7 days, so that the animals were able to make the association of moving bars with the presence of the platform. For the experimental conditions during trainings, a bar pattern with maximum contrast (100%) and optimal spatial frequency (0.088 cycles/degree) was used. In the experiment, the contrast and spatial frequency parameters were modified until the mice were unable to see the bar pattern. Contrast sensitivity values were measured, and the results of treated and untreated mice were compared. FIG. 5 shows that the greatest differences were observed in the outlier values of spatial frequency, where there is greater difficulty in recognizing the pattern of moving bars (0.01; 0.02; 0.17 and 0.35 cycles/degree).

### Example 80. In Vitro Efficacy of Compound 35 in Preventing Phototoxicity of 661W Cells

661W is a mouse photoreceptor-derived cell line (661W) immortalized by the expression of simian virus (SV) T antigen (T-ag) under control of the human IRBP promoter. Cellular and molecular analyses show that these cells express cone but not rod photoreceptor markers, which suggests that the cells arise from a cone photoreceptor lineage. For this reason, the 661W cell line should contribute significantly to the study of cone photoreceptors cell function and of diseases affecting cone photoreceptor cells, including mechanisms of photoreceptor cell death in various retinal dystrophies, including age-related macular degeneration, Stargardt's disease or diabetic retinopathy among others (Tan et al. Invest Ophthalmol. Vis. Sci., 2004, 45, 764).

661 W cells were seeded in a 96-wells impedance plate (50,000 cells/well), previously coated with ECM 1:100, and grown O/N at 37° C. and 5% CO₂ in DMEM/F-12 (Thermo Fisher, 11320033) supplemented with 40 μL/L hydrocortisone 21-hemisuccinate (Sigma, H-2270), 40 μL/L progesterone (Sigma, P-8783), 0.032 g/L putrescine (Sigma, P-7505), 40 μL/L β-mercaptoethanol (Sigma, M-6250), antibiotic-antimycotic solution (Sigma, A5955) and 10% fetal bovine serum (FBS). After 8 h, the medium was replaced by DMEM/F-12 without FBS or complements to remove cellular debris. When cells arrived at a plateau in the impedance recording (4-8 hours after the medium change, depending on the culture plate) compound 35 was added and two hours later the 9-cis-Retinal (Cf=20 μM) was added. The cells were placed in the Maestro Z and exposed to 30,000 white light luxes.

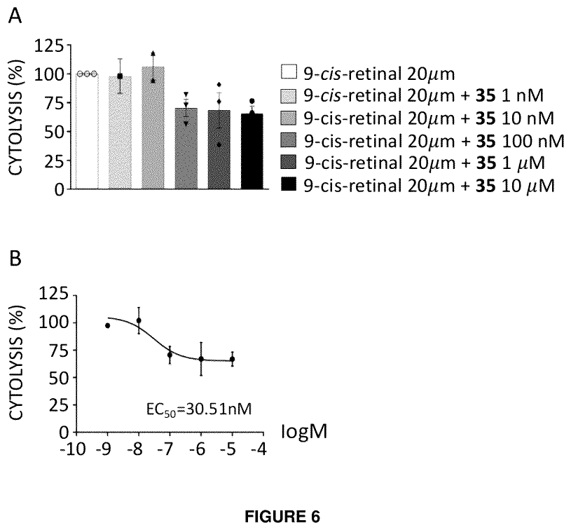

to register the impedance. FIG. 6 shows that compound 35 protects against 9-cis-Retinal and light-induced phototoxicity of 661W cells in a dose-response manner with a potency of EC50=30.5 nM.

1. A method for the prevention or treatment of a retinal disease, said method comprising administering to a patient in need thereof of a therapeutically effective amount of a compound of formula (I): 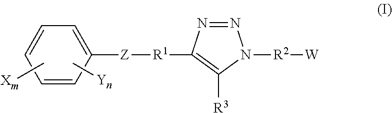
wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, CN, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
R¹ is —CH₂—;
R² is a linear C₁-C₄ alkylene biradical;
R³ is H; and
W is selected from the group consisting of —NH₂, —NH(C₁-C₄ alkyl), —N(C₁-C₄ alkyl)₂, —N(C₁-C₄ alkyl)₃, 1-pyrrolidinyl optionally substituted by a C₁-C₄ alkyl, 1-piperidinyl optionally substituted by a C₁-C₄ alkyl, 4-morpholinyl optionally substituted by a C₁-C₄ alkyl, 4-piperazinyl optionally substituted by a C₁-C₄ alkyl, —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, —N(C₁-C₄ alkyl)C(═NH)NHC(═NH)NH₂, —CO₂H, and —SO₃H;
or a pharmaceutically acceptable salt, stereoisomer, tautomer, solvate or N-oxide thereof, or an isotopically labeled derivative thereof.

2. The method according to claim 1, wherein in the compound of formula (I): m is 1; n is 0; X is selected from the group consisting of O(C₁-C₄ alkyl), C₁-C₆ alkyl and CF₃; R¹ is —CH₂—; R² is selected from the group consisting of —CH₂—CH₂— and —CH₂—CH₂—CH₂—; R³ is H; and W is selected from the group consisting of —N(C₁-C₄ alkyl)₂, 1-piperidinyl, 4-morpholinyl, 1-pyrrolidinyl, and 4-piperazinyl optionally substituted by a C₁-C₄ alkyl.

3. The method according to claim 1, wherein in the compound of formula (I): Z is —S—; R¹ is —CH₂—; and R³ is H.

4. The method according to claim 1, wherein in the compound of formula (I): m is 1; n is 0; and X is in para position with respect to Z.

5. The method according to claim 1, wherein the compound of formula (I) is selected from the group consisting of:
1-[2-(N,N-dimethylamino)ethyl]-4-[(4-methoxyphenoxy)methyl]-1H-1,2,3-triazole;
1-[2-(N,N-dimethylamino)ethyl]-4-[(3,4-methylendioxyphenoxy)methyl]-1H-1,2,3-triazole;
1-[2-(N,N-dimethylamino)ethyl]-4-[(3-methoxyphenoxy)methyl]-1H-1,2,3-triazole;
4-[(3,5-dimethoxyphenoxy)methyl]-1-[2-(N,N-dimethylamino)ethyl]-1H-1,2,3-triazole;
1-[2-(N,N-dimethylamino)ethyl]-4-(phenoxymethyl)-1H-1,2,3-triazole;
1-[2-(N,N-dimethylamino)ethyl]-4-[(3-trifluoromethylphenoxy)methyl]-1H-1,2,3-triazole;
1-[2-(N,N-dimethylamino)ethyl]-4-[(3,4,5-trimethoxyphenoxy)methyl]-1H-1,2,3-triazole;
4-[(3,4-dimethoxyphenoxy)methyl]-1-[2-(N,N-dimethylamino)ethyl]-1H-1,2,3-triazole;
1-[2-(N,N-dimethylamino)ethyl]-4-[1-(4-methoxyphenoxy)-1-(methyl)-ethyl]-1H-1,2,3-triazole;
4-[(4-methoxyphenoxy)methyl]-1-[2-(N-methylamino)ethyl]-1H-1,2,3-triazole;
1-(2-aminoethyl)-4-[(4-methoxyphenoxy)methyl]-1H-1,2,3-triazole;
1-(2-aminoethyl)-4-[(3-methoxyphenoxy)methyl]-1H-1,2,3-triazole;
1-(2-aminoethyl)-4-[(3,4-methylendioxyphenoxy)methyl]-1H-1,2,3-triazole;
1-(2-aminoethyl)-4-(phenoxymethyl)-1H-1,2,3-triazole;
4-[(4-methoxyphenoxy)methyl]-1-[2-(pyrrolidin-1-yl)ethyl]-1H-1,2,3-triazole;
4-[(4-methoxyphenoxy)methyl]-1-[2-(morpholin-4-yl)ethyl]-1H,1,2,3-triazole;
4-[(4-methoxyphenoxy)methyl]-1-[2-(piperidin-1-yl)ethyl]-1H-1,2,3-triazole;
1-[2-(piperidin-1-yl)ethyl]-4-[(3-trifluoromethylphenoxy)methyl]-1H-1,2,3-triazole;
1-[2-(piperidin-1-yl)ethyl]-4-[(4-trifluoromethylphenoxy)methyl]-1H-1,2,3-triazole;
1-[2-(piperidin-1-yl)ethyl]-4-[(2-trifluoromethylphenoxy)methyl]-1H-1,2,3-triazole;
(guanidyl)ethyl]-4-[(4-methoxyphenoxy)methyl]-1H-1,2,3-triazole;
1-[2-(guanidyl)ethyl]-4-[(3-methoxyphenoxy)methyl]-1H-1,2,3-triazole;
1-[2-(guanidyl)ethyl]-4-[(3,4-methylendioxyphenoxy)methyl]-1H-1,2,3-triazole;
1-[2-(guanidyl)ethyl]-4-(phenoxymethyl)-1H-1,2,3-triazole;
1-[2-(biguanidyl)ethyl]-4-[(4-methoxyphenoxy)methyl]-1H-1,2,3-triazole;
1-[2-(biguanidyl)ethyl]-4-[(3-methoxyphenoxy)methyl]-1H-1,2,3-triazole;
1-[2-(biguanidyl)ethyl]-4-[(3,4-methylendioxyphenoxy)methyl]-1H-1,2,3-triazole;
1-[2-(biguanidyl)ethyl]-4-(phenoxymethyl)-1H-1,2,3-triazole;
1-[2-(N,N-dimethylamino)ethyl]-4-[(4-hydroxyphenoxy)methyl]-1H-1,2,3-triazole;
1-[2-(N,N-dimethyl)-N-oxideaminoethyl]-4-[(4-methoxyphenoxy)methyl]-1H-1,2,3-triazole;
1-[2-(N,N-dimethylamino)ethyl]-4-[(4-methoxyphenyl)thiomethyl]-1H-1,2,3-triazole;
1-[3-(N,N-dimethylamino)propyl]-4-[(4-methoxyphenyl)thiomethyl]-1H-1,2,3-triazole;
1-[2-(N,N-diethylamino)ethyl]-4-[(4-methoxyphenyl)thiomethyl]-1H-1,2,3-triazole;
1-[2-(piperidin-1-yl)ethyl]-4-[(3-(trifluoromethylphenyl)thiomethyl]-1H-1,2,3-triazole;
4-[(4-methoxyphenyl)thiomethyl]-1-[2-(piperidin-1-yl)ethyl]-1H-1,2,3-triazole;
4-[(4-tert-butylphenyl)thiomethyl]-1-[2-(piperidin-1-yl)ethyl]-1H-1,2,3-triazole;
4-[(4-isopropylphenyl)thiomethyl]-1-[2-(4-morpholinyl)ethyl]-1H,1,2,3-triazole;
1-[2-(4-morpholinyl)ethyl]-4-(phenylthiomethyl)-1H,1,2,3-triazole;
1-[2-(N,N-dimethylamino)ethyl]-4-[(4-hydroxyphenyl)thiomethyl]-1H-1,2,3-triazole;
1-[1-(R)-benzyl-2-(piperidin-1-yl)ethyl]-4-[(4-methoxyphenyl)thiomethyl]-1H-1,2,3-triazole;
1-[1-(R)-benzyl-2-(piperidin-1-yl)ethyl]-4-[(3-trifluoromethylphenyl)thiomethyl]-1H-1,2,3-triazole;
1-[1-(R)-benzyl-2-(piperidin-1-yl)ethyl]-4-[(4-tert-butylphenyl)thiomethyl]-1H-1,2,3-triazole;
1-[2-(guanidyl)ethyl]-4-[(4-methoxyphenyl)thiomethyl]-1H-1,2,3-triazole;
1-[2-(biguanidinyl)ethyl]-4-[(4-methoxyphenyl)thiomethyl]-1H-1,2,3-triazole;
1-[3-(biguanidinyl)propyl]-4-[(4-methoxyphenyl)thiomethyl]-1H-1,2,3-triazole;
1-[2-(N,N-dimethylamino)ethyl]-5-trithium-4-[(4-methoxyphenyl)thiomethyl]-1H-1,2,3-triazole;
1-[2-(N,N-dimethylamino)ethyl]-4-[(4-methoxyphenyl)sulfinylmethyl]-1H-1,2,3-triazole;
1-[3-(N,N-dimethylamino)propyl]-4-[(4-methoxyphenyl)sulfinylmethyl]-1H-1,2,3-triazole;
1-[2-(N,N-diethylamino)ethyl]-4-[(4-methoxyphenyl)sulfinylmethyl]-1H-1,2,3-triazole;
1-[2-(piperidin-1-yl)ethyl]-4-[(3-trifluoromethylphenyl)sulfinylmethyl]-1H-1,2,3-triazole;
4-[(4-isopropylphenyl)sulfinylmethyl]-1-[2-(4-morpholinyl)ethyl]-1H,1,2,3-triazole;
4-[(2,6-dimethylphenyl)sulfinylmethyl]-1-[2-(piperidin-1-yl)ethyl]-1H-1,2,3-triazole;
1-[2-(4-morpholinyl)ethyl]-4-(phenylsulfinylmethyl)-1H,1,2,3-triazole;
4-[(4-methoxyphenyl)sulfinylmethyl]-1-[2-(piperidin-1-yl)ethyl]-1H-1,2,3-triazole;
1-[1-(R)-benzyl-2-(piperidin-1-yl)ethyl]-4-[(3-trifluoromethylphenyl)sulfinylmethyl]-1H-1,2,3-triazole;
1-[2-(N,N-dimethylamino)ethyl]-4-[(4-hydroxyphenyl)sulfinylmethyl]-1H-1,2,3-triazole;
1-(2-aminoethyl)-4-[(4-methoxyphenyl)sulfinylmethyl]-1H-1,2,3-triazole;
1-[2-(N-methylamino)-ethyl]-4-[(4-methoxyphenyl)sulfinylmethyl]-1H-1,2,3-triazole;
1-[2-(N,N-dimethyl)-N-oxideaminoethyl]-4-[(4-methoxyphenyl)sulfinylmethyl]-1H-1,2,3-triazole;
1-[2-(N,N-dimethylamino)ethyl]-4-[(4-methoxyphenyl)sulfonylmethyl]-1H-1,2,3-triazole;
1-[2-(N,N-diethylamino)ethyl]-4-[(4-bromophenyl)sulfonylmethyl]-1H-1,2,3-triazole;
1-[2-(N,N-dimethylamino)ethyl]-4-[(3,4-dimethoxyphenyl)sulfonylmethyl]-1H-1,2,3-triazole;
4-[(4-isopropylphenyl)sulfonylmethyl]-1-[2-(4-morpholinyl)ethyl]-1H,1,2,3-triazole;
4-(phenylsulfonylmethyl)-1-[2-(4-morpholinyl)ethyl]-1H,1,2,3-triazole;
1-[2-(N,N-dimethylamino)ethyl]-4-[(3-trifluoromethylphenyl)sulfonylmethyl]-1H-1,2,3-triazole;
1-(2-aminoethyl)-4-[(4-methoxyphenyl)sulfonylmethyl]-1H-1,2,3-triazole; and
1-[2-(N-methylamino)-ethyl]-4-[(4-methoxyphenyl)sulfonylmethyl]-1H-1,2,3-triazole.

6. A method for the prevention or treatment of a retinal disease, said method comprising administering to a patient in need thereof of a therapeutically effective amount of a pharmaceutical composition comprising a compound of formula (I) or a pharmaceutically acceptable salt, stereoisomer, tautomer, solvate or N-oxide thereof, or an isotopically labeled derivative thereof as defined in claim 1 and a pharmaceutically acceptable excipient.

7. The method according to claim 1, wherein the retinal disease is selected from the group consisting of retinitis pigmentosa, diabetic retinopathy, cone dystrophies, rod and cone degeneration, Leber congenital amaurosis, retinitis Punctata albescens, choroideremia, gyrate atrophy of the choroid and retina, choroidal dystrophy, X-linked retinoschisis, Wagner vitreoretinal degeneration, autosomal dominant vitreoretinochoroidopathy, Stargardt disease, Best vitelliform macular dystrophy; dominant vitreoretinochoroidopathy; Usher syndrome, Bardet-Biedl syndrome, Sorsby's fundus dystrophy, age-related macular degeneration, autosomal dominant congenital stationary night blindness, achromatopsia and macular drusen.

8. The method according to claim 7, wherein the retinal disease is selected from retinitis pigmentosa, Stargardt disease, Leber congenital amaurosis, age-related macular degeneration and diabetic retinopathy.

9. The method according to claim 7, wherein the retinal disease is selected from retinitis pigmentosa, autosomal dominant congenital stationary night blindness and achromatopsia.

10. The method according to claim 1, wherein the compound of formula (I), or a pharmaceutical composition comprising the compound of formula (I), is administered topically, intraocularly, orally, parenterally, transdermally, intranasally, or rectally.

11. The method according to claim 1, wherein the compound of formula (I), or a pharmaceutical composition comprising the compound of formula (I), is administered topically on the corneal surface, preferably in the form of drops, gel, cream, spray or contact lenses.

12. The method according to claim 1, wherein the compound of formula (I) is administered at a dose of 0.001 to 10 mg/day.

13. A compound of formula (II): 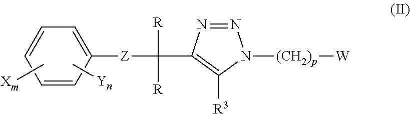
wherein
X and Y are independently selected from the group consisting of C₁-C₆ alkyl, OH, O(C₁-C₄ alkyl), OCF₃, S(C₁-C₄ alkyl), NHC(O)(C₁-C₄ alkyl), CF₃, F, Cl, Br and I; or X and Y together represent a methylenedioxy or ethylenedioxy biradical;
m and n are independently selected from the group consisting of 0, 1, 2, 3 and 4;
Z is selected from the group consisting of —S—, —S(═O)—, —S(═O)₂— and —O—;
each R is H;
R³ is H;
when Z is —S—, —S(═O)— or —S(═O)₂—, p is selected from the group consisting of 2, 3 and 4, and W is selected from the group consisting of —NHC(═NH)NH₂, —NHC(═NH)NH(C₁-C₄ alkyl), —NHC(═N(C₁-C₄ alkyl)NH(C₁-C₄ alkyl), —NHC(═NH)NHC(═NH)NH₂, and —N(C₁-C₄ alkyl)C(═NH)NHC(═NH)NH₂; and
when Z is —O—, p is 2, and W is selected from the group consisting of —NH(CH₃), —N(CH₃)₂, 1-piperidinyl, and 4-morpholinyl;
or a pharmaceutically acceptable salt, stereoisomer, tautomer, solvate or N-oxide thereof, or an isotopically labeled derivative thereof.

14. The compound according to claim 13, wherein when Z is —O—,
m is selected from the group consisting of 0, 1, 2 and 3;
n is selected from the group consisting of 0, 1 and 2;
X, when present, is in meta or para position with respect to Z and is selected from the group consisting of OH, O(C₁-C₄ alkyl), OCF₃ and CF₃;
Y, when present, is in ortho position with respect to Z and is CF₃;
or X and Y together represent a methylenedioxy or ethylenedioxy biradical;
each R is H;
R³ is H;
p is 2;
and
W is selected from the group consisting of —NH(CH₃), —N(CH₃)₂, 1-piperidinyl, and 4-morpholinyl.

15. The compound of formula (II) according to claim 13, wherein:
wherein m is 1; n is 0;
Y is selected from the group consisting of C₁-C₆ alkyl, OH, O(C₁-C₄ alkyl), OCF₃, S(C₁-C₄ alkyl), NHC(O)(C₁-C₄ alkyl), CF₃, F, Cl, Br and I;
X is in para position with respect to Z;
X is O(C₁-C₄ alkyl);
Z is —S— or —O—;
when Z is —S—, p is selected from the group consisting of 2 and 3;
when Z is —O—, p is 2;
R is H;
R³ is H;
when Z is —S— W is NHC(═NH)NHC(═NH)NH₂; and
when Z is —O— W is —N(C₁-C₄ alkyl)₂.

16. (canceled)

17. A pharmaceutical composition comprising a compound of formula (II) or a pharmaceutically acceptable salt, stereoisomer, tautomer, solvate or N-oxide thereof, or an isotopically labeled derivative thereof according to claim 13 and a pharmaceutically acceptable excipient.

18. The method according to claim 6, wherein the pharmaceutically acceptable excipient is selected from the group consisting of hyaluronic acid, carboxymethylcellulose, carbomer and mixtures thereof.

19. The pharmaceutical composition according to claim 17, wherein the pharmaceutically acceptable excipient is selected from the group consisting of hyaluronic acid, carboxymethylcellulose, carbomer and mixtures thereof.
